(19) United States
(12) Patent Application Publication
(10) Pub. No.: US 2009/0112193 A1
(43) Pub. Date: Apr. 30, 2009

(54) Systems and devices that utilize photolyzable nitric oxide donors

(75) Inventors: Roderick A. Hyde, Redmond, WA (US); Muriel Y. Ishikawa, Livermore, CA (US); Lowell L. Wood, JR., Bellevue, WA (US)

Correspondence Address: Searete LLC;Suite 110, 1756 - 114th Ave. S.E., Bellevue, WA, 98004, US

(73) Assignee: Searete LLC, a limited liability corporation of the State of Delaware

(21) Appl. No.: 12/148,284

(22) Filed: Apr. 16, 2008

Related U.S. Application Data
(63) Continuation-in-part of application No. 11/998,864, filed on Nov. 30, 2007.
Application No. 11/998,864 is a continuation-in-part of application No. 11/981,743, filed on Oct. 30, 2007.

(51) Int. Cl. A61B 18/18

(52) U.S. Cl. 606/11

(57) ABSTRACT

The present disclosure relates to systems and devices that utilize photolyzable nitric oxide donors.

### CROSS-REFERENCE TO RELATED APPLICATIONS

The present application is related to and claims the benefit of the earliest available effective filing date(s) from the following listed application(s) (the “Related Applications”) (e.g.; claims earliest available priority dates for other than provisional patent applications or claims benefits under 35 USC §119(e) for provisional patent applications, for any and all parent, grandparent, great-grandparent, etc.-applications of the Related Application(s)).

### RELATED APPLICATIONS

For purposes of the USPTO extra-statutory requirements, the present application constitutes a continuation-in-part of U.S. patent application Ser. No. 11/981,743, entitled Methods and Systems for Use of Photolyzable Nitric Oxide Donors, naming Roderick A. Hyde as inventor, filed 30 Oct. 2007, which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week11/patbene.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary and/or admission as to whether or not the present application contains any new matter in addition to the matter of its parent application(s).

All subject-matter of the Related Applications and of any and all parent, grandparent, great-grandparent, etc. applications of the Related Applications is incorporated herein by reference to the extent such subject matter is not inconsistent herewith.

### TECHNICAL FIELD

The present disclosure relates to systems and devices that utilize photolyzable nitric oxide donors.

### SUMMARY

In some embodiments one or more devices are provided that include one or more photolyzable nitric oxide donors and one or more light sources that are physically associated with the one or more photolyzable nitric oxide donors. The device may optionally include one or more nitric oxide permeable housings. The device may optionally include one or more control units. The device may optionally include one or more nitric oxide sensors. The device may optionally include one or more nitric oxide sensors. In addition to the foregoing, other aspects are described in the claims, drawings, and text forming a part of the present disclosure.

In some embodiments one or more systems are provided that include circuitry for operating one or more light sources that are physically associated with one or more photolyzable nitric oxide donors. The system may optionally include circuitry for operating one or more nitric oxide permeable housings. The system may optionally include circuitry for operating one or more control units. The system may optionally include circuitry for operating one or more nitric oxide sensors. The system may optionally include circuitry for operating one or more nitric oxide permeable housings.

In addition to the foregoing, other aspects are described in the claims, drawings, and text forming a part of the present disclosure.

In some embodiments one or more systems are provided that include means for operating one or more light sources that are physically associated with one or more photolyzable nitric oxide donors. The system may optionally include means for operating one or more nitric oxide permeable housings. The system may optionally include means for operating one or more control units. The system may optionally include means for operating one-or more nitric oxide sensors. The system may optionally include means for operating one or more nitric oxide permeable housings. In addition to the foregoing, other aspects are described in the claims, drawings, and text forming a part of the present disclosure.

In some embodiments one or more systems are provided that include a signal-bearing medium bearing one or more instructions for operating one or more light sources that are physically associated with one or more photolyzable nitric oxide donors. The system may optionally include one or more instructions for operating one or more nitric oxide permeable housings. The system may optionally include one or more instructions for operating one or more control units. The system may optionally include one or more instructions for operating one or more nitric oxide sensors. The system may optionally include one or more instructions for operating one or more nitric oxide permeable housings. The system may optionally include one or more instructions for operating one or more nitric oxide sensors. In addition to the foregoing, other aspects are described in the claims, drawings, and text forming a part of the present disclosure.

In some embodiments, means include but are not limited to circuitry and/or programming for effecting the herein referenced functional aspects; the circuitry and/or programming can be virtually any combination of hardware, software, and/or firmware configured to effect the herein referenced functional aspects depending upon the design choices of the system designer. In addition to the foregoing, other system aspects means are described in the claims, drawings, and/or text forming a part of the present disclosure.

In some embodiments, related systems include but are not limited to circuitry and/or programming for effecting the herein referenced method aspects; the circuitry and/or programming can be virtually any combination of hardware, software, and/or firmware configured to effect the herein referenced method aspects depending upon the design choices of the system designer. In addition to the foregoing, other system aspects are described in the claims, drawings, and/or text forming a part of the present application.

The foregoing summary is illustrative only and is not intended to be in any way limiting. In addition to the illustrative aspects, embodiments, and features described above, further aspects, embodiments, and features will become apparent by reference to the drawings, claims, and the following detailed description.

### BRIEF DESCRIPTION OF THE FIGURES

FIG. 1 illustrates an example system 100 in which embodiments may be implemented.

FIG. 2 illustrates embodiment 200 of device 102 within system 100.

FIG. 3 illustrates alternate embodiments of embodiment 200 of device 102 within system 100.

FIG. 4 illustrates alternate embodiments of embodiment 200 of device 102 within system 100.

FIG. 4a illustrates alternate embodiments of embodiment 200 of device 102 within system 100.

FIG. 5 illustrates alternate embodiments of embodiment 200 of device 102 within system 100.

FIG. 6 illustrates embodiment 600 of device 102 within system 100.

FIG. 7 illustrates alternate embodiments of embodiment 600 of device 102 within system 100.

FIG. 8 illustrates embodiment 800 of device 102 within system 100.

FIG. 9 illustrates alternate embodiments of embodiment 800 of device 102 within system 100.

FIG. 10 illustrates alternate embodiments of embodiment 800 of device 102 within system 100.

FIG. 11 illustrates alternate embodiments of embodiment 800 of device 102 within system 100.

FIG. 12 illustrates embodiment 1200 of device 102 within system 100.

FIG. 13 illustrates alternate embodiments of embodiment 1200 of device 102 within system 100.

FIG. 13a illustrates alternate embodiments of embodiment 1200 of device 102 within system 100.

FIG. 14 illustrates alternate embodiments of embodiment 1200 of device 102 within system 100.

FIG. 15 illustrates embodiment 1500 of device 102 within system 100.

FIG. 16 illustrates alternate embodiments of embodiment 1500 of device 102 within system 100.

FIG. 17 illustrates embodiment 1700 of device 102 within system 100.

FIG. 18 illustrates alternate embodiments of embodiment 1700 of device 102 within system 100.

FIG. 19 illustrates alternate embodiments of embodiment 1700 of device 102 within system 100.

FIGS. 20A-20E illustrate embodiments of device 102.

FIGS. 21A-2D illustrate embodiments of device 102.

FIGS. 22A-22C illustrate embodiments of device 102.

FIGS. 23A-23D illustrate embodiments of device 102.

FIGS. 24A-24C illustrate embodiments of device 102.

FIGS. 25A-25C illustrate embodiments of device 102.

FIGS. 26A-26C illustrate embodiments of device 102.

FIGS. 27A-27D illustrate embodiments of nitric oxide permeable housing 114.

FIGS. 28A-28C illustrate embodiments of nitric oxide permeable housing 114.

FIG. 29 illustrates a partial view of a system 2900 that includes a computer program for executing a computer process on a computing device.

FIG. 30 illustrates a partial view of a system 3000 that includes a computer program for executing a computer process on a computing device.

FIG. 31 illustrates a partial view of a system 31 00 that includes a computer program for executing a computer process on a computing device.

FIG. 32 illustrates a partial view of a system 3200 that includes a computer program for executing a computer process on a computing device.

FIG. 33 illustrates a partial view of a system 3300 that includes a computer program for executing a computer process on a computing device.

FIG. 34 illustrates a partial view of a system 3400 that includes a computer program for executing a computer process on a computing device.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

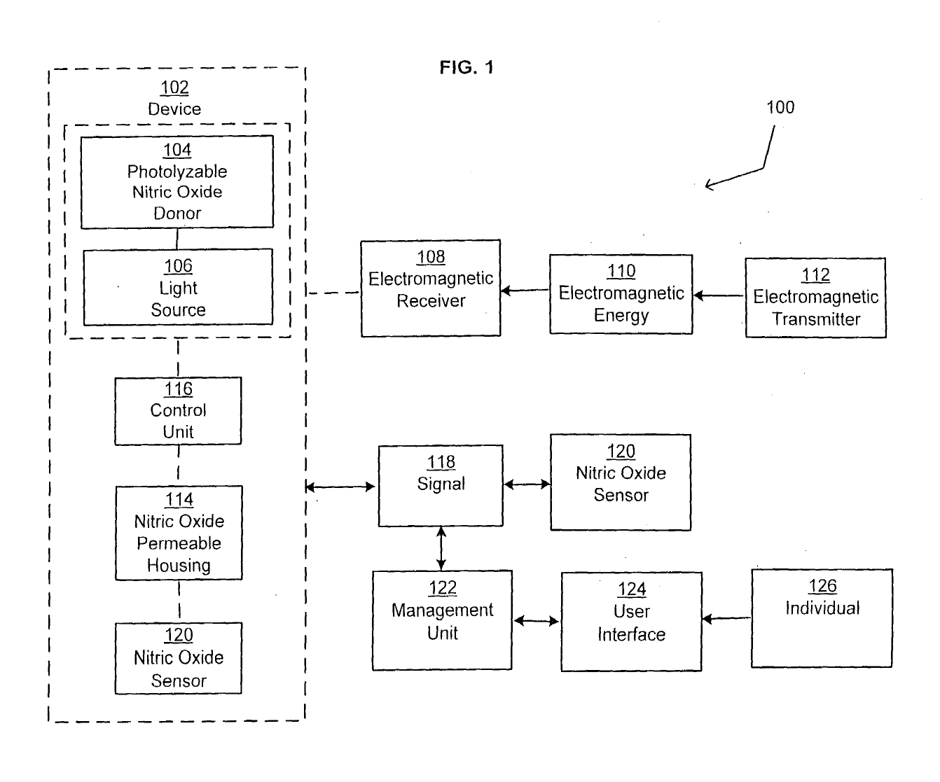

FIG. 1 illustrates a system 100 in which embodiments may be implemented. System 100 may include one or more devices 102 that include one or more light sources 106 and one or more photolyzable nitric oxide donors 104. In some embodiments, system 100 may include one or more control units 116, one or more nitric oxide permeable housings 114, one or more nitric oxide sensors 120, and substantially any combination thereof. In some embodiments, the photolyzable nitric oxide donors 104 may be physically coupled with the one or more light sources 106. For example, in some embodiments, the one or more light sources 106 may be coated with the one or more photolyzable nitric oxide donors 104. In some embodiments, the one or more light sources 106 may include one or more polymeric materials that are coupled to at least one of the photolyzable nitric oxide donors 104. In some embodiments, one or more light sources 106 may be coated with a composition that includes one or more photolyzable nitric oxide donors 104. In some embodiments, one or more light sources 106 may be included within a housing that is coated with one or more photolyzable nitric oxide donors 104. Accordingly, in some embodiments, one or more light sources 106 may be in direct contact with one or more photolyzable nitric oxide donors 104. In some embodiments, one or more light sources 106 may be in indirect contact with one or more photolyzable nitric oxide donors 1-04. In some embodiments, the device 102 may include one or more operably coupled control units 116. In some embodiments, the one or more control units 116 may be operably coupled to the one or more light sources 106. In some embodiments, the one or more control units 116 may be operably coupled to the one or more light sources 106 and may be used to control the operation of the one or more light sources 106. In some embodiments, the one or more control units 116 may be configured to receive one or more signals 118. In some embodiments, the one or more control units 116 nay be configured to receive one or more signals 118 from one or more transmitters. In some embodiments, the one or more control units 116 may be configured to receive one or more signals 118 from one or more nitric oxide sensors 120. In some embodiments, one or more nitric oxide permeable housings 114 may be configured to enclose one or more photolyzable nitric oxide donors 104. In some embodiments, one or more nitric oxide permeable housings 114 may be configured to enclose one or more photolyzable nitric oxide donors 104 and one or more light sources 106. In some embodiments, one or more nitric oxide permeable housings 114 may be configured to enclose one or more photolyzable nitric oxide donors 104, one or more light sources 106, and one or more control units 116. In some embodiments, one or more nitric oxide permeable housings 114 may be configured to enclose one or more photolyzable nitric oxide donors 104, one or more light sources 106, one or more control units 116, and one or more nitric oxide sensors 120. In some embodiments, one or more nitric oxide permeable housings 114 may be configured to enclose one or more photolyzable nitric oxide donors 104, one or more light sources 106, one or more control units 116, one or more nitric oxide sensors 120, or substantially any combination thereof. In some embodiments, one or more devices may be operably coupled to one or more electromagnetic receivers 108. In some embodiments, system 100 may include one or more electromagnetic receivers 108 that are configured to receive electromagnetic energy. In some embodiments, system 100 may include one or more electromagnetic receivers 108 that are configured to receive electromagnetic energy 110 that is transmitted by one or more electromagnetic transmitters 112. In some embodiments, the one or more electromagnetic receivers 108 may be operably coupled to the device. In some embodiments, the one or more electromagnetic receivers 108 may be operably coupled to the one or more light sources 106. In some embodiments, the one or more electromagnetic receivers 108 may be operably coupled to the one or more light sources 106 such that the one or more light sources 106 are energized through receipt of electromagnetic energy. In some embodiments, system 100 may include one or more light sources 106, one or more photolyzable nitric oxide donors 104, one or more control units 16, one or more nitric oxide permeable housings 114, one or more nitric oxide sensors 120, one or more electromagnetic receivers 108, one or more electromagnetic transmitters 112, or substantially an), combination thereof.

### Device

System 100 includes one or more devices 102. A device 102 may be configured in numerous ways. In some embodiments, a device 102 may be configured for implantation into an individual 126. For example, in some embodiments, a device 102 may be configured for implantation into the genital region of an individual 126. In some embodiments, a device 102 may be configured for application to an inside surface of an individual 126. For example, in some embodiments, a device 102 may be configured for insertion into the urethra of an individual 126. In some embodiments, a device 102 may be configured for vaginal insertion into an individual 126. In some embodiments, a device 102 may be configured for application to an outside surface of an individual 126. For example, in some embodiments, a device 102 may be configured for application to the skin of an individual 126. Accordingly, a device 102 may be configured in numerous ways to deliver nitric oxide to a surface or region of an individual 126. In some embodiments, a device 102 may be configured to deliver nitric oxide as a therapeutic agent. In some embodiments, a device 102 may be configured to deliver nitric oxide as a sanitizing agent. For example, in some embodiments, a device 102 may be configured to deliver nitric oxide to the surface of a table, a chair, to surgical instruments, and the like. In some embodiments, a device 102 may be incorporated into clothing. For example, in some embodiments, one or more devices 102 may be incorporated into a glove, a mitten, a hood, a mask, a sock, a shirt, a sheet, a bandage, tape, and the like.

### Light Source

Numerous light sources 106 may be used within system 100. In some embodiments, one or more light sources 106 may be used to facilitate release of nitric oxide from one or more photolyzable nitric oxide donors 104. In some embodiments, one or more light sources 106 may be configured to emit light of multiple wavelengths. In some embodiments, one or more light sources 106 may be configured to emit light that is selected to facilitate release of nitric oxide from one or more photolyzable nitric oxide donors 104. For example, in some embodiments, one or more light sources 106 may be configured to emit one or more wavelengths of light that are selected to facilitate release of nitric oxide from one or more identified photolyzable nitric oxide donors 104. In some embodiments, one or more light sources 106 may emit one or more wavelengths of light that are selected based on the absorption spectrum of one or more photolyzable nitric oxide donors 104. In some embodiments, one or more light sources 106 may emit one or more wavelengths of light that are selected based on decomposition of one or more photolyzable nitric oxide donors 104. For example, in some embodiments, one or more light sources 106 may be configured to emit one or more wavelengths of light that cause decomposition of one or more photolyzable nitric oxide donors 104 without causing injury to adjacent structures and/or tissues. In some embodiments, a first light source 106 may be configured to emit one or more wavelengths of light that cause a first photolyzable nitric oxide donor 104 to release nitric oxide and a second light source 106 may be configured to emit one or more wavelengths of light that cause a second photolyzable nitric oxide donor 104 to release nitric oxide. Accordingly, numerous light sources 106 may be coupled with numerous types of photolyzable nitric oxide donors 104 to provide for selective release of nitric oxide.

In some embodiments, one or more light sources 106 may include one or more quantum dots (e.g., U.S. Pat. No. 7,235,361; herein incorporated by reference). For example, in some embodiments, one or more light sources 106 may be configured to emit one or more wavelengths of light that are absorbed by one or more quantum dots. In some embodiments, one or more quantum dots may be configured to absorb light and then emit one or more wavelengths of light that cause release of nitric oxide from one or more photolyzable nitric oxide donors 104. Accordingly, in some embodiments, emission from one or more first quantum dots may be tuned to facilitate release of nitric oxide from one or more first photolyzable nitric oxide donors 104 and emission from one or more second quantum dots may be tuned to facilitate release of nitric oxide from one or more second photolyzable nitric oxide donors 104.

In some embodiments, one or more light sources 106 may be configured to be used internally to illuminate one or more regions of an individual 126. A light source 106 may be configured in numerous ways. For example, in some embodiments, one or more light sources 106 may be configured for insertion into the urethra of a male and/or a female (e.g., U.S. Pat. No. 4,248,214; herein incorporated by reference). In some embodiments, one or more light sources 106 may be configured for vaginal insertion into a female. In some embodiments, one or more light sources 106 may be configured for implantation into an individual 126. For example, in some embodiments, one or more light sources 106 may be configured for implantation into the genital region of a male and/or a female. For example, in some embodiments, one or more light sources 106 may be configured for implantation within the corpus cavernosa of a penis. In some embodiments, one or more light sources 106 may be configured for implantation into the scrotal sack of a male. For example, in some embodiments, one or more light sources 106 may be configured to include one or more energy sources (e.g., one or more batteries), one or more light emitters (e.g., one or more light emitting diodes), and one or more optical fibers to deliver light to a selected region of an individual 126. In some embodiments, such light sources 106 may be implanted such that the energy sources and the light emitters are implanted into the scrotal sack of a male and optical fibers may be operably coupled to the one or more light emitters and implanted within the corpus cavernosa of the associated penis.

In some embodiments, one or more light sources 106 may be configured to externally illuminate an individual 126. Accordingly, one or more light sources 106 may be configured in numerous ways. For example, in some embodiments, a light source 106 may be associated with a lamp, a flashlight, a wand, a ring, a glove, a sheet, a condom, and the like. In some embodiments, one or more light sources 106 may be associated with clothing.

In some embodiments, light sources 106 may be remotely controlled. For example, in some embodiments, one or more light sources 106 may be configured to receive one or more signals 118 that include instructions for operation of the one or more light sources 106. Such instructions may be associated with emission of light, non-emission of light, time when light is emitted, length of light emission, intensity of light emission, wavelengths of emitted light, and the like.

In some embodiments, light sources 106 may be configured to include one or more control units 116. In some embodiments, one or more light sources 106 may be configured to include a switch that may be used to turn the light source 106 on and off. For example, in some embodiments; a light source 106 may be configured to include a push button switch to turn the light source 106 on and off.

In some embodiments, one or more light sources 106 may include one or more light emitters that are coupled to one or more electromagnetic receivers 108. The one or more electromagnetic receivers 108 may be configured to couple with one or more electromagnetic transmitters 112 that produce one or more electromagnetic fields that induce an electrical current to flow in the one or more electromagnetic receivers 108 to energize the light emitters (e.g., U.S. Pat. No. 5,571,152; herein incorporated by reference). Accordingly, in some embodiments, one or more light sources 106 may be configured such that they are not directly coupled to an energy source.

A light source 106 may be configured to emit numerous types of light. In some embodiments, emitted light may be visible light. In some embodiments, emitted light may be infrared light. In some embodiments, emitted light may be ultraviolet light. In some embodiments, emitted light may be substantially any combination of visible light, infrared light, and/or ultraviolet light. In some embodiments, one or more light sources 106 may emit fluorescent light. In some embodiments, one or more light sources 106 may emit phosphorescent light.

In some embodiments, one or more light sources 106 may be configured to emit light continuously. In some embodiments, one or more light sources 106 may be configured to emit light as a pulse. In some embodiments, one or more light sources 106 may be configured to emit light as a flash. In some embodiments, one or more light sources 106 may be configured to emit light continuously, as a pulse, as a flash, or substantially any combination thereof.

In some embodiments, one or more light emitters and/or light sources 106 may be configured to provide for upconversion of energy. In some embodiments, infrared light may be upconverted to visible light (e.g., Mendioroz et al., Optical Materials, 26:351-357 (2004). In some embodiments, infrared light may be upconverted to ultraviolet light (e.g., Mendioroz et al., Optical Materials, 26:351-357 (2004). In some embodiments, one or more light sources 106 may include one or more rare-earth materials (e.g., ytterbium-erbium, ytterbium-thulium, or the like) that facilitate upconversion of energy (e.g., U.S. Pat. No. 7,088,040; herein incorporated by reference). For example, in some embodiments, one or more light sources 106 may be associated with Nd³⁺ doped KPb₂Cl₅ crystals. In some embodiments, one or more light sources 106 may be associated with thliogallates doped with rare earths, such as CaGa₂S₄:Ce³⁺ and SrGa₂S₄:Ce³⁺. In some embodiments, one or more light sources 106 may be associated with aluminates that are doped with rare earths, such as YAlO₃:Ce³⁺, YGaO₃:Ce³⁺, Y(Al,Ga)O₃:Ce³⁺, and orthosilicates M₂SiO₅:Ce³⁺ (M:Sc, Y, Sc) doped with rare earths, such as, for example, Y₂SiO₅:Ce³⁺. In some embodiments, yttrium may be replaced by scandium or lanthanum (e.g., U.S. Pat. Nos. 6,812,500 and 6,327,074; herein incorporated by reference). Numerous materials that may be used to upconvert energy have been described (e.g., U.S. Pat. Nos. 5,956,172; 5,943,160; 7,235,189; 7,215;687; herein incorporated by reference).

### Photolyzable Nitric Oxide Donor/Nitric Oxide

Numerous photolyzable nitric oxide donors 104 may be used within system 100. Examples of such photolyzable nitric oxide donors 104 include, but are not limited to, diazelliumdiolates (e.g., U.S. Pat. Nos. 7,105,502; 7,122,529; 6,673,338; herein incorporated by reference), trans-[RuCl([15]aneN4)NO]+2 (Ferezin et al., Nitric Oxide, 13:170-175 (2005), Bonaventura et al., Nitric Oxide, 10:83-91 (2004)), nitrosyl ligands (e.g., U.S. Pat. No. 5,665,077; herein incorporated by reference, Chmura et al., Nitric Oxide, 1-5:370-379 (2005), Flitney et al., Br. J. Pharmacol., 107:842-848 (1992), Flitney et al., Br. J. Pharmacol., 117:1549-1557 (1996), Matthews et al., Br. J. Pharmacol., 113:87-94 (1994)), 6-Nitrobenzo[a]pyrene (e.g., Fukuhara et al., J. Am. Chem. Soc., 123:8662-8666 (2001)), S-nitroso-glutathione (e.g., Rotta et al., Braz. J. Med. Res., 36:587-594 (2003), Flitney and Megson, J. Physiol., 550:819-828 (2003)), S-nitrosothiols (e.g., Andrews et al., British Journal of Pharmacology, 138:932-940 (2003), Singh et al., FEBS Lett., 360:47-51 (1995)), 2-Methyl-2-nitrosopropane (e.g., Pou et al., Mol. Pharm., 46:709-715 (1994), Wang et al., Chem. Rev., 102:1091-1134 (2002)), imidazolyl derivatives (e.g., U.S. Pat. No. 5,374,710; herein incorporated by reference).

In some embodiments, one or more photolyzable nitric oxide donors 104 may be used in association with additional nitric oxide donors that are not photolyzable. In some embodiments, one or more photolyzable nitric oxide donors 104 may be used in association with additional agents. Examples of such additional agents include, but are not limited to, enzyme inhibitors (e.g., U.S. Pat. No. 6,943,166; herein incorporated by reference), agents that increase the effects and/or concentration of nitric oxide 106 (e.g., methylene blue and N(w)-nitro-L-arginine (L-NOARG) (see Chen and Gillis, Biochem. Biophys. Res. Commun., 190, 559-563 (1993) and Kim et al., J. Vet. Sci., 1:81-86 (2000)), L-arginine (e.g., U.S. Published Patent Application No.: 20020068365 and U.S. Pat. No. 6,635,273; herein incorporated by reference), agents that stabilize nitric oxide donors (e.g., dimethly sulfoxide and ethanol), agents that increase the half life of nitric oxide (e.g., U.S. Published Patent Application No.: 20030039697; herein incorporated by reference), and the like.

### Control Unit

Numerous types of control units 116 may be used within system 100. In some embodiments, one or more control units 116 may be operably coupled with one or more light sources 106, one or more nitric oxide sensors 120, one or more electromagnetic receivers 108, one or more electromagnetic transmitters 112, or substantially any combination thereof. In some embodiments, one or more control units 116 may be operably coupled to other components through use of one or more wireless connections, one or more hardwired connections, or substantially any combination thereof. Control units 116 may be configured in numerous ways. For example, in some embodiments, a control unit 116 may be configured as an on/off switch. Accordingly, in some embodiments, a control unit 116 may be configured to turn a light source on and/or off. In some embodiments, a control unit 116 may be configured to control the emission of light from one or more light sources 106. For example, in some embodiments, one or more control units 116 may regulate the intensity of light emitted from one or more light sources 106, the duration of light emitted from one or more light sources 106, the frequency of light emitted from one or more light sources 106, wavelengths of light emitted from one or more light sources 106, or substantially any combination thereof. In some embodiments, one or more control units 116 may be configured to receive one or more signals 118 from one or more nitric oxide sensors 120. Accordingly, in some embodiments, one or more control units 116 may be configured to control one or more light sources 106 in response to one or more signals 118 received from one or more nitric oxide sensors 120. For example, in some embodiments, one or more nitric oxide sensors 120 may sense a low concentration of nitric oxide in one or more tissues and send one or more signals 118 to one or more control units 116. The one or more control units 116 may then turn one or more light sources 106 on to facilitate release of nitric oxide from one or more photolyzable nitric oxide donors 104. Accordingly, in some embodiments, one or more nitric oxide sensors 120 may sense a high concentration of nitric oxide in one or more tissues and send one or more signals 118 to one or more control units 116. The one or more control units 116 may then turn one or more light sources 106 off to end release of nitric oxide from one or more photolyzable nitric oxide donors 104. In some embodiments, one or more control units 116 may be programmed to control one or more light sources 106. For example, in some embodiments, one or more control units 116 may be programmed to turn one or more light sources 106 on for a predetermined amount of time and then turn off. Accordingly, in some embodiments, one or more control units 116 may be preprogrammed. In some embodiments, one or more control units 116 may be dynamically programmed. For example, in some embodiments, one or more management units 122 may receive one or more signals 118 from one or more nitric oxide sensors 120 and program one or more control units 116 in response to the one or more signals 118 received from the one or more nitric oxide sensors 120. In some embodiments, one or more control units 116 may include one or more receivers that are able to receive one or more signals 118, one or more information packets, or substantially any combination thereof. Control units 116 may be configured in numerous ways. For example, in some embodiments, one or more control units 116 may be operably coupled to one or more light sources 106 that include numerous light emitting diodes that emit light of different wavelengths. Accordingly, in some embodiments, one or more control units 116 may control the wavelengths of light emitted by the one or more light sources 106 by controlling the operation of light emitting diodes that emit light of the selected wavelength. Accordingly, control units 116 may be configured in numerous ways and utilize numerous types of mechanisms.

### Nitric Oxide Permeable Housing

Numerous types of nitric oxide permeable housings 114 may be used within system 100. Nitric oxide permeable housings 114 may be configured for implantation within an individual 126. In some embodiments, one or more nitric oxide permeable housings 114 may be configured for implantation into the genital region of an individual 126. For example, in some embodiments, one or more nitric oxide permeable housings 114 maybe configured for implantation into the corpus cavernosa of a penis. Nitric oxide permeable housings 114 may be configured to facilitate application of nitric oxide to a surface. In some embodiments, one or more nitric oxide permeable housings 114 may be configured to facilitate application of nitric oxide to one or more surfaces of an individual 126. For example, in some embodiments, one or more nitric oxide permeable housings 114 may be configured as a canister having a nitric oxide permeable end that may be positioned on a skin surface of an individual 126 to deliver nitric oxide to the skin surface. In some embodiments, one or more nitric oxide permeable housings 114 may be configured for insertion into the urethra of a male. In some embodiments, one or more nitric oxide permeable housings 114 may be configured for insertion into the vagina of a female.

In some embodiments, one or more nitric oxide permeable housings 114 may be configured to enclose at least a portion of one or more photolyzable nitric oxide donors 104. In some embodiments, one or more nitric oxide permeable housings 114 may be configured to enclose at least a portion of one or more light sources 106. In some embodiments, one or more nitric oxide permeable housings 114 may be configured to enclose at least a portion of one or more control units 116. In some embodiments, one or more nitric oxide permeable housings 114 may be configured to enclose at least a portion of one or more nitric oxide sensors 120. In some embodiments, one or more nitric oxide permeable housings 114 may be configured to enclose one or more photolyzable nitric oxide donors 104. In some embodiments, one or more nitric oxide permeable housings 114 may be configured to enclose one or more light sources 106. In some embodiments, one or more nitric oxide permeable housings 114 may be configured to enclose one or more photolyzable nitric oxide donors 104 and one or more light sources 106. In some embodiments, one or more nitric oxide permeable housings 114 may be configured to enclose one or more photolyzable nitric oxide donors 104, one or more light sources 106, and one or more control units 116. In some embodiments, one or more nitric oxide permeable housings 114 may be configured to enclose one or more photolyzable nitric oxide donors 104, one or more light sources 106, one or more control units 116, and one or more electromagnetic receivers 108. In some embodiments, one or more nitric oxide permeable housings 114 may be configured to enclose one or more photolyzable nitric oxide donors 104, one or more light sources 106, one or more control units 116, one or more electromagnetic receivers 108, or substantially any combination thereof. In some embodiments, one or more nitric oxide permeable housings 114 may be configured to include one or more compartments. For example, in some embodiments, a nitric oxide permeable housing 114 may include a compartment that is configured to accept one or more light sources 106 and a second compartment that is configured to accept one or more photolyzable nitric oxide donors 104. In some embodiments, a nitric oxide permeable housing 114 may include a compartment that is configured to accept one or more light sources 106, a light permeable divider, and a second compartment that is configured to accept one or more photolyzable nitric oxide donors 104. In some embodiments, a light permeable divider may be made of a material that allows light to pass through the divider. Examples of such material include, but are not limited to, plastic, quartz, and the like.

Nitric oxide permeable housings 114 may be constructed of numerous types of materials and combinations of materials. Examples of such materials include, but are not limited to, ceramics, polymeric materials, metals, plastics, and the like. In some embodiments, nitric oxide permeable housings 114 may include numerous combinations of materials. For example, in some embodiments, a nitric oxide permeable housing 114 may include a nitric oxide impermeable metal canister that is coupled to a nitric oxide permeable membrane (e.g., U.S. Patent Application No.: 20020026937). In some embodiments, a nitric oxide permeable housing 114 may include a selectively permeable membrane. For example, in some embodiments, a nitric oxide permeable housing 114 may include a selectively permeable, hydrophilic polyester co-polymer membrane system that includes a copolymer with 70% polyester and 30% polyether (e.g., Sympatex™ 10 μm membrane, see Hardwick et al., Clinical Science, 100:395-400 (2001)). In some embodiments, a nitric oxide permeable housing 114 may include a scintered glass portion that is permeable to nitric oxide. Accordingly, nitric oxide permeable housings 114 may include numerous types of porous ceramics that are permeable to nitric oxide. In some embodiments, a nitric oxide permeable housing 114 may include a nitric oxide permeable coating (e.g., U.S. Patent Application Nos.: 20050220838; 20030093143). In some embodiments, one or more nitric oxide permeable housing 114 may include one or more valves. In some embodiments, a nitric oxide permeable housing 114 may include one or more valves that are controllable by one or more control units 116. Accordingly, in some embodiments, valves may be opened or closed in response to one or more nitric oxide sensors 120, one or more signals 118, one or more information packets, one or more management units 122, or substantially any combination thereof.

### Nitric Oxide Sensor

Numerous types of nitric oxide sensors 120 may be used within system 100. In some embodiments, a nitric oxide sensor 120 may be configured for implantation into an individual 126. For example, in some embodiments, one or more nitric oxide sensors 120 may be configured to be implanted into the genital region of an individual 126. Accordingly, in some embodiments, one or more nitric oxide sensors 120 may be used to determine the presence of nitric oxide in one or more tissues. In some embodiments, a nitric oxide sensor 120 may be configured for use on the outside surface of an individual 126. For example, in some embodiments, one or more nitric oxide sensors 120 may be configured to detect the concentration of nitric oxide on the surface of skin, a wound, a surface of a table, and the like. In some embodiments, one or more nitric oxide sensors 120 may be configured to be included within one or more housings. In some embodiments, one or more nitric oxide sensors 120 may be configured to be included within one or more nitric oxide permeable housings 114. In some embodiments, a nitric oxide sensor 120 may be configured to utilize fluorescence to detect nitric oxide. For example, in some embodiments, a nitric oxide sensor may detect nitric oxide through use of one or more fluorescent probes, such as 4,5-diaminofluorescein diacetate (EMD Chemicals Inc., San Diego, Calif.). In some embodiments, a nitric oxide sensor may detect nitric oxide through use of one or more electrodes. For example, in some embodiments, a nitric oxide sensor may utilize an electrode that includes a single walled carbon nanotube and an ionic liquid to detect nitric oxide (e.g., Li et al., Electroanalysis, 18:713-718 (2006)). Numerous nitric oxide sensors 120 are commercially available and have been described (e.g., World Precision Instruments, Inc., Sarasota, Fla., USA; U.S. Pat. Nos. 6,100,096; 6,280,604; 5,980,705). In some embodiments, a nitric oxide sensor 120 may include one or more transmitters. In some embodiments, a nitric oxide sensor 120 may include one or more receivers. In some embodiments, a nitric oxide sensor 120 may be configured to transmit one or more signals 118. In some embodiments, a nitric oxide sensor 120 may be configured to receive one or more signals 118.

### Electromagnetic Receiver

Numerous types of electromagnetic receivers 108 may be used within system 100. In some embodiments, one or more electromagnetic receivers 108 may be used to electromagnetically couple power to energize one or more light sources 106 from an external power supply. Methods to construct such electromagnetic receivers 108 have been described (e.g., U.S. Pat. No. 5,571,152). Briefly, in some embodiments, one or more electromagnetic receivers 108 may be associated with one or more rectifier chips. The one or more electromagnetic receivers 108 may include one or more cores about which are wrapped an electrical conductor. In some embodiments, cores may comprise a material, such as a ferrite material, due to its relatively high magnetic permeability and low magnetic hysteresis. However, other materials can be used for this purpose. In some embodiments, the electromagnetic receiver 108 may be operably coupled to a light emitting diode.

### Electromagnetic Transmitter

Numerous types of electromagnetic transmitters 112 may be used within system 100. Methods to construct electromagnetic transmitters 112 have been described (e.g., U.S. Pat. No. 5,571,152). Briefly, in some embodiments, the electromagnetic transmitter 112 may include a ferrite core around which is wrapped an electrical conductor. Other types of material having high magnetic permeability and relatively low magnetic hysteresis may be used for the core. Insulating tape may be wrapped around the electrical conductor, or the electromagnetic transmitter 112 may be dipped in a resin to form a coating that stabilizes and fixes the electrical conductor on the core. A return lead from one end of the electrical conductor may include one of two leads that are coupled to an AC power supply.

### Electromagnetic Energy

Electrical power may be electromagnetically coupled from one or more electromagnetic transmitters 112 with one or more electromagnetic receivers 108. Accordingly, electrical power that is transferred to the one or more electromagnetic receivers 108 may be used to power one or more operably linked light emitters. Methods and devices that may be used to transmit electrical power to a light emitter have been described (e.g., U.S. Pat. No. 5,571,152).

### Management Unit

In some embodiments, system 100 may include one or more management units 122. In some embodiments, a management unit 122 may be configured as a computer. Accordingly, in some embodiments, a management unit 122 may be configured to accept input and provide output. For example, in some embodiments, a management unit 122 may receive one or more signals 118 from one or more nitric oxide sensors 120, process the one or more signals 118, and then transmit one or more signals 118. In some embodiments, one or more transmitted signals 118 may be received by one or more control units 116. In some embodiments, one or more transmitted signals 118 may be received by one or more light sources 106. Accordingly, in some embodiments, a management unit 122 may be configured to manage nitric oxide production by a device. For example, in some embodiments, a management unit 122 may include and execute a set of instructions for the operation of one or more control-units 116 that facilitate production of nitric oxide by one or more devices 102 at preselected times and for preselected concentrations. In some embodiments, such production may be regulated through control of the intensity of light emitted by one or more light sources 106, the duration of light emitted by one or more light sources 106, the frequency of light emitted by one or more light sources 106, and the like. In some embodiments, a management unit 122 may dynamically control the production of nitric oxide by one or more devices. For example, in some embodiments, a management unit 122 may be configured to maintain a nitric oxide concentration within a range of concentrations. Accordingly, the management unit 122 may receive one or more signals 118 from one or more nitric oxide sensors 120 indicating a current concentration of nitric oxide. The management unit 122 may then determine if the nitric oxide concentration is within a range of nitric oxide concentrations or out of a range of nitric oxide concentrations and then increase nitric oxide production, decrease nitric oxide production, or maintain nitric oxide production to cause the nitric oxide concentration to be maintained within a range. Accordingly, a management unit 122 may be used in numerous ways to regulate nitric oxide production.

### Transmitter

The system 100 may include one or more transmitters. In some embodiments, one or more transmitters may be operably coupled to one or more nitric oxide sensors 120. In some embodiments, one or more transmitters may be operably coupled to one or more management units 122. In some embodiments, one or more transmitters may be operably coupled to one or more control units 116. In some embodiments, one or more transmitters may be operably coupled to one or more nitric oxide sensors 120, one or more control units 116, one or more management units 122, or substantially any combination thereof. Numerous types of transmitters may be used in association with system 100. Examples of such transmitters include, but are not limited to, transmitters that transmit one or more optical signals 118, radio signals 118, wireless signals 118, hardwired signals 118, infrared signals 118, ultrasonic signals 118, and the like (e.g., U.S. Pat. Nos. RE39,785; 7,260,768; 7,260,764; 7,260,402; 7,257,327; 7,215,887; 7,218,900; herein incorporated by reference). In some embodiments, one or more transmitters may transmit one or more signals 118 that are encrypted. Numerous types of transmitters are known and have been described (e.g., U.S. Patent Nos. and Published U.S. Patent Applications: U.S. Pat. Nos. 7,236,595; 7,260,155; 7,227,956; US2006/0280307; herein incorporated by reference).

### Signal

Numerous types of signals 118 may be used in association with system 100. Examples of such signals 118 include, but are not limited to, optical signals 118, radio signals 118, wireless signals 118, hardwired signals 118, infrared signals 118, ultrasonic signals 118, and the like.

In some embodiments, one or more signals 118 may not be encrypted. In some embodiments, one or more signals 118 may be encrypted. In some embodiments, one or more signals 118 may be sent through use of a secure mode of transmission. In some embodiments, one or more signals 118 may be coded for receipt by a specific individual 126. In some embodiments, such code may include anonymous code that is specific for all individual 126. Accordingly, information included within one or more signals 118 may be protected against being accessed by others who are not the intended recipient.

### Receiver

System 100 may include one or more receivers. In some embodiments, one or more receivers may be operably coupled to one or more nitric oxide sensors 120. In some embodiments, one or more receivers may be operably coupled to one or more management units 122. In some embodiments, one or more receivers may be operably coupled to one or more control units 116. In some embodiments, one or more receivers may be operably coupled to one or more nitric oxide sensors 120, one or more control units 116, one or more management units 122, or substantially any combination thereof. Numerous types of receivers may be used in association with system 100. Examples of such receivers include, but are not limited to, receivers that receive one or more optical signals 118, radio signals 118, wireless signals 118, hardwired signals 118, infrared signals 118, ultrasonic signals 118, and the like. Such receivers are known and have been described (e.g., U.S. Pat. Nos. RE39,785; 7,218,900; 7,254,160; 7,245,894; 7,206,605; herein incorporated by reference).

### User Interface/User

System 100 may include numerous types of user interfaces 124. For example, one or more users (e.g., individuals 126) may interact through use of numerous user interfaces 124 that utilize hardwired methods, such as through use of an on/off switch, a push button, a keyboard, and the like. In some embodiments, the user interface 124 may utilize wireless methods, such as methods that utilize a transmitter and receiver, utilize the internet, and the like.

### Individual

A device 102 may be used to deliver nitric oxide to an individual 126. In some embodiments, an individual 126 may be a human. In some embodiments, an individual 126 may be a human male. In some embodiments, an individual 126 may be a human female. A device 102 may be used within numerous contexts. For example, in some embodiments, a device 102 may be used to deliver nitric oxide to an individual 126 to treat sexual dysfunction. In some embodiments, a device 102 may be used to treat female arousal disorder. In some embodiments, a device 102 may be used to treat male erectile disorder. In some embodiments, sexual dysfunction may be due to a physical condition. For example, in some embodiments, sexual dysfunction may result from surgery, a physical injury, pharmaceutical use, age, or the like. In some embodiments, sexual dysfunction may be due to a mental condition. For example, in some embodiments, sexual dysfunction may be due to depression, lack of interest, insecurity, anxiety, or the like. In some embodiments, a device 102 may deliver nitric oxide to increase sexual performance and/or pleasure. In some embodiments, a device 102 may be used to deliver nitric oxide to the skin of an individual 126. In some embodiments, such delivery may be for cosmetic purposes. In some embodiments, such delivery may be for therapeutic purposes. For example, in some embodiments, a device 102 may be used to deliver nitric oxide to a skin lesion, such as a skin ulcer, a burn, a cut, a puncture, a laceration, a blunt trauma, an acne lesion, a boil, and the like. In some embodiments, a device 102 may be used to deliver nitric oxide to a skin surface to increase the expression of endogenous collagenase. In some embodiments, a device 102 may be used to deliver nitric oxide to a skin surface to regulate the formation of collagen. In some embodiments, a device 102 may be used to deliver nitric oxide to reduce inflammation (e.g., reduce exudate secretion) at the site of a lesion (e.g., U.S. Patent Application No.: 2007/0088316). In some embodiments, a device 102 may be used to deliver nitric oxide to reduce the microbial burden within a wound site. For example, in some embodiments, a device 102 may be used to deliver nitric oxide as an antibacterial agent against methicillin-resistant Staphylococcus aureus. A device 102 may deliver nitric oxide to an individual 126 at numerous concentrations. For example, in some embodiments, nitric oxide may be delivered at a concentration ranging from about 160 ppm to about 400 ppm. Such concentrations may be used without inducing toxicity in the healthy cells around a wound site (e.g., U.S. Patent Application No.: 2007/0088316).

### Administration Form

Numerous types of administration forms 110 may be used to provide one or more photolyzable nitric oxide donors 104 to an individual 126. In some embodiments, an administration form may be a formulation of one or more photolyzable nitric oxide donors 104. In some embodiments, an administration form may be configured for oral delivery of one or more photolyzable nitric oxide donors 104 to an individual 126. For example, in some embodiments, an administration form may be configured as a pill, a lozenge, a capsule, a liquid, and the like. In some embodiments, an administration form may be configured for topical delivery of one or more photolyzable nitric oxide donors 104 to an individual 126. For example, in some embodiments, an administration form may be configured as a gel, a cream, a lotion, a lubricant, a jelly, and the like. In some embodiments, one or more photolyzable nitric oxide donors 104 may be formulated with one or more liposomes to provide for delivery of the one or more photolyzable nitric oxide donors 104 to the individual 126. In some embodiments, one or more photolyzable nitric oxide donors 104 may be formulated with one or more detergents to facilitate delivery of the one or more photolyzable nitric oxide donors 104 to the individual 126. In some embodiments, one or more photolyzable nitric oxide donors 104 may be formulated with one or more agents that stabilize the one or more photolyzable nitric oxide donors 104. In some embodiments, one or more photolyzable nitric oxide donors 104 may be formulated for administration to one or more individuals 126 through inhalation. In some embodiments, one or more photolyzable nitric oxide donors 104 may be formulated for administration to one or more individuals 126 through parenteral administration.

In some embodiments, an administration form may include an implant. In some embodiments, one or more photolyzable nitric oxide donors 104 may be coupled to a structure that can be implanted within an individual 126. For example, in some embodiments, one or more photolyzable nitric oxide donors 104 may be coupled to a polymeric structure for implantation into an individual 126 (e.g., U.S. Pat. Nos. 5,405,919; 6,451,337; 7,052,711: herein incorporated by reference, Smith et al., J. Med. Chem., 1:1148-1156 (1996)). In some embodiments, one or more photolyzable nitric oxide donors 104 may be included within a porous structure and/or matrix for implantation into an individual 126 (e.g., U.S. Published Patent Application No.: 20030039697; herein incorporated by reference). Such structures may be constructed from numerous materials that include, but are not limited to, polymers, ceramics, metals, and the like. In some embodiments, one or more photolyzable nitric oxide donors 104 may be formulated for depot administration to an individual 126. For example, in some embodiments, one or more photolyzable nitric oxide donors 104 may be formulated with one or more biodegradable materials that degrade within an individual 126 to release the one or more photolyzable nitric oxide donors 104 (e.g., U.S. Pat. Nos. 5,736,152; 6,143,314; 6,773,714; herein incorporated by reference). Accordingly, in some embodiments, one or more photolyzable nitric oxide donors 104 may be included within a flowable material that forms an implant upon being injected into an individual 126.

In some embodiments, one or more photolyzable nitric oxide donors 104 may be formulated with one or more additional agents. Examples of such agents include, but are not limited to, enzyme inhibitors, additional nitric oxide donors, free radical scavengers, and the like. In some embodiments, one or more photolyzable nitric oxide donors 104 may be formulated with one or more light sources 106 (e.g., U.S. Pat. No. 5,571,152; herein incorporated by reference). In some embodiments, one or more photolyzable nitric oxide donors 104 may be formulated with one or more quantum dots (e.g., U.S. Pat. No. 7,235,361; herein incorporated by reference).

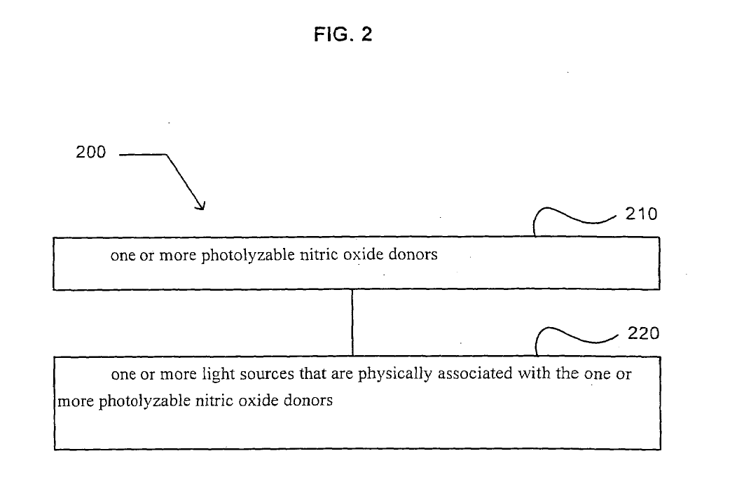

FIG. 2 illustrates embodiment 200 of device 102 within system 100. In FIG. 2, discussion and explanation may be provided with respect to the above-described example of FIG. 1, and/or with respect to other examples and contexts. However, it should be understood that the modules may execute operations in a number of other environments and contexts, and/or modified versions of FIG. 1. Also, although the various modules are presented in the sequence(s) illustrated, it should be understood that the various modules may be configured in numerous orientations.

The embodiment 200 may include module 210 that includes one or more photolyzable nitric oxide donors. In some embodiments, device 102 may include one or more photolyzable nitric oxide donors 104 that release nitric oxide upon photolysis.

Examples of such photolyzable nitric oxide donors 104 include, but are not limited to, diazeniumdiolates (e.g., U.S. Pat. Nos. 7,105,502; 7,122,529; 6,673,338; herein incorporated by reference), trans-[RuCl([15]aneN4)NO]+2 (Ferezin et al., Nitric Oxide, 13:170-175 (2005), Bonaventura et al., Nitric Oxide, 10:83-91 (2004)), nitrosyl ligands (e.g., U.S. Pat. No. 5,665,077; herein incorporated by reference, Chmura et al., Nitric Oxide, 15:370-379 (2005), Flitney et al., Br. J. Pharmacol., 107:842-848 (1992), Flitney et al., Br. J. Pharmacol., 117:1549-1557 (1996), Matthews et al., Br. J. Pharmacol., 113:87-94 (1994)), 6-Nitrobenzo[a]pyrene (e.g., Fukuhara et al., J. Am. Chem. Soc., 123:8662-8666 (2001)), S-nitroso-glutathione (e.g., Rotta et al., Braz. J. Med. Res., 36:587-594 (2003), Flitney and Megson, J. Physiol., 550:819-828 (2003)), S-nitrosothiols (e.g., Andrews et al., British Journal of Pharmacology, 138:932-940 (2003), Singh et al., FEBS Lett., 360:47-51 (1995)), 2-Methyl-2-nitrosopropane (e.g., Pou et al., Mol. Pharm., 46:709-715 (1994), Wang et al., Chem. Rev., 102:1091-1134 (2002)), imidazolyl derivatives (e.g., U.S. Pat. No. 5,374,710; herein incorporated by reference).

The embodiment 200 may include module 220 that includes one or more light sources that are physically associated with the one or more photolyzable nitric oxide donors. In some embodiments, device 102 may include one or more light sources 106 that are physically associated with one or more photolyzable nitric oxide donors 104. In some embodiments, the one or more light sources 106 may be directly coupled to one or more photolyzable nitric oxide donors 104. For example, in some embodiments, the one or more photolyzable nitric oxide donors 104 may be chemically coupled to a surface of the light source 106 (e.g., chemically coupled to a polymer coating on the light source). In some embodiments, one or more photolyzable nitric oxide donors 104 may be indirectly coupled to one or more light sources 106. For example, in some embodiments, one or more photolyzable nitric oxide donors 104 may be coupled to a material that is used to coat the one or more light sources 106.

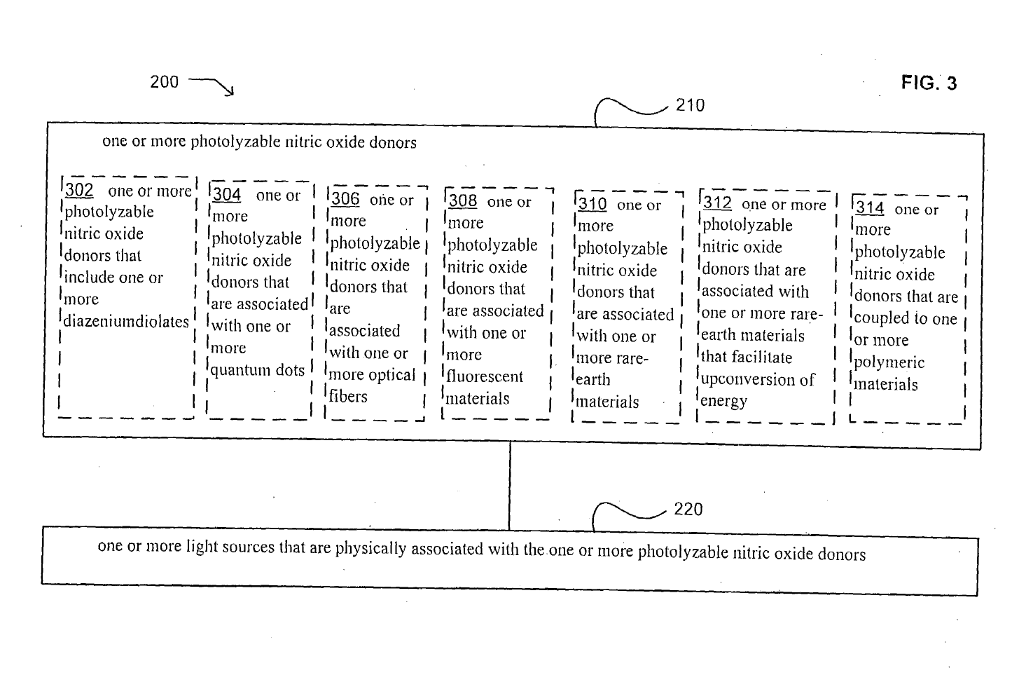

FIG. 3 illustrates alternative embodiments of embodiment 200 of device 102 within system 100 of FIG. 2. FIG. 3 illustrates example embodiments of module 210. Additional embodiments may include an embodiment 302, an embodiment 304, an embodiment 306, an embodiment 308, an embodiment 310, an embodiment 312, and/or an embodiment 314.

At embodiment 302, module 210 may include one or more photolyzable nitric oxide donors that include one or more diazeniumdiolates. In some embodiments, one or more photolyzable nitric oxide donors 104 may include one or more diazeniumdiolates. Many photolyzable nitric oxide donors 104 that are diazeniumdiolates are known and have been described (e.g., U.S. Pat. No. 7,122,529). Examples of such diazeniumdiolates include, but are not limited to, O²-benzyl, O²-naphthylmethyl substituted diazeniumdiolates and O²-naphthylallyl substituted diazeniumndiolates.

At embodiment 304, module 210 may include one or more photolyzable nitric oxide donors that are associated with one or more quantum dots. In some embodiments, one or more photolyzable nitric oxide donors 104 may be associated with one or more quantum dots. For example, in some embodiments, one or more diazeniumdiolates may be associated with one or more quantum dots. In some embodiments, one or more quantum dots may be tuned to emit light that facilitates photolysis of one or more nitric oxide donors. In some embodiments, a quantum dot may be tuned to emit light that specifically facilitates photolysis of one or more nitric oxide donors. For example, in some embodiments, one or more quantum dots may emit select wavelengths of light that correspond to wavelengths of light that cause photolysis of one or more nitric oxide donors. In some embodiments, one or more quantum dots may be selected that absorb light emitted by one or more light sources 106 and emit light that facilitates photolysis of one or more nitric oxide donors.

At embodiment 306, module 210 may include one or more photolyzable nitric oxide donors that are associated with one or more optical fibers. In some embodiments, one or more photolyzable nitric oxide donors 104 may be associated with one or more optical fibers. In some embodiments, one or more photolyzable nitric oxide donors 104 may be directly associated with one or more optical fibers. For example, in some embodiments, one or more optical fibers may be directly coated with one or more photolyzable nitric oxide donors 104. In some embodiments, one or more optical fibers may be directly coated with one or more compositions that include one or more photolyzable nitric oxide donors 104.

In some embodiments, one or more portions of one or more optical fibers may be directly coated with one or more photolyzable nitric oxide donors 104. In some embodiments, one or more portions of one or more optical fibers may be directly coated with one or more compositions that include one or more photolyzable nitric oxide donors 104. In some embodiments, one or more photolyzable nitric oxide donors 104 may be indirectly associated with one or more optical fibers. For example, in some embodiments, one or more optical fibers may be inserted into a structure that is coated with one or more photolyzable nitric oxide donors 104. In some embodiments, one or more optical fibers may be inserted into a structure that is coated with one or more compositions that include one or more photolyzable nitric oxide donors 104. In some embodiments, one or more optical fibers may be inserted into a structure that is partially coated with one or more photolyzable nitric oxide donors 104. In some embodiments, one or more optical fibers may be inserted into a structure that is partially coated with one or more compositions that include one or more photolyzable nitric oxide donors 104. For example, in some embodiments, one or more optical fibers may be inserted into one or more tubes that are coated with one or more photolyzable nitric oxide donors 104. In some embodiments, one or more optical fibers may be inserted into one or more tubes that are coated with one or more compositions that include one or more photolyzable nitric oxide donors 104. One or more photolyzable nitric oxide donors 104 may be associated with numerous types of optical fibers. Methods to construct optical fibers have been described. Examples of optical fibers include, but are not limited to, optical fibers that include a single core and/or one or more cores. In some embodiments, an optical fiber may include silica glass. In some embodiments, an optical fiber may include a cladding. Optical fibers have been described (e.g., U.S. Pat. Nos. 7,295,741; 7,295,737).

At embodiment 308, module 210 may include one or more photolyzable nitric oxide donors that are associated with one or more fluorescent materials. In some embodiments, one or more light sources 106 may include one or more photolyzable nitric oxide donors 104 that are associated with one or more fluorescent materials. Numerous fluorescent materials may be associated with one or more light sources 106. Examples of such materials include, but are not limited to, 1,4-diphenylbutadiyne; 9,10-diphenylanthracene; benzene; biphenyl; ethyl-p-dimethylaaminobenzoate; naphthalene; P-terphenyl; ethyl-p-dimethylaminobenzoate; stilbene; tryptophan; tyrosine; 1,2-diphenylacetylene; 7-methoxycoumarin-4-acetic acid; anthracene; indo-1; POPOP; P-quaterphenyl; pyrene; and the like.

At embodiment 310, module 210 may include one or more photolyzable nitric oxide donors that are associated with one or more rare-earth materials. In some embodiments, one or more light sources 106 may include one or more photolyzable nitric oxide donors 104 that are associated with one or more rare-earth materials. In some embodiments, one or more rare-earth materials may include one or more rare-earth elements. The rare-earth elements are a collection of sixteen chemical elements in the periodic table, namely scandium, yttrium, and fourteen of the fifteen lanthanoids (excluding promethium). In some embodiments, one or more rare-earth materials may include one or more rare-earth elements that fluoresce.

At embodiment 312, module 210 may include one or more photolyzable nitric oxide donors that are associated with one or more rare-earth materials that facilitate upconversion of energy. In some embodiments, one or more photolyzable nitric oxide donors 104 may be associated with one or more rare-earth materials that facilitate upconversion of energy. In some embodiments, infrared light may be upconverted to visible light (e.g., Mendioroz et al., Optical Materials, 26:351-357 (2004). In some embodiments, infrared light may be upconverted to ultraviolet light (e.g., Mendioroz et al., Optical Materials, 26:351-357 (2004). In some embodiments, one or more photolyzable nitric oxide donors 104 may be associated with one or more rare-earth materials (e.g., ytterbium-erbium, ytterbium-thulium, or the like) that facilitate upconversion of energy (e.g., U.S. Pat. No. 7,088,040; herein incorporated by reference). For example, in some embodiments, one or more photolyzable nitric oxide donors 104 may be associated with Nd³⁺ doped KPb₂Cl₅ crystals. In some embodiments, one or more photolyzable nitric oxide donors 104 may be associated with thiogallates doped with rare earths, such as CaGa₂S₄:Ce³⁺ and SrGa₂S₄:Ce³⁺. In some embodiments, one or more photolyzable nitric oxide donors 104 may be associated with aluminates that are doped with rare earths, such as YAlO₃:Ce³⁺, YGaO₃:Ce³⁺, Y(Al,Ga)O₃:Ce³⁺, and orthosilicates M₂SiO₅ :Ce³⁺ (M:Sc, Y, Sc) doped with rare earths, such as, for example, Y₂SiO₅:Ce³⁺. In some embodiments, yttrium may be replaced by scandium or lanthanum (e.g., U.S. Pat. Nos. 6,812,500 and 6,327,074; herein incorporated by reference). Numerous materials that may be used to upconvert energy have been described (e.g., U.S. Pat. Nos. 5,956,179; 5,943,160; 7,935,189 7,215,687; herein incorporated by reference).

At embodiment 314, module 210 may include one or more photolyzable nitric Oxide donors that are coupled to one or more polymeric materials. In some embodiments, one or more photolyzable nitric oxide donors 104 may be coupled to one or more polymeric materials. For example, in some embodiments, one or more polymer matrices may be impregnated with one or more photolyzable nitric oxide donors 104 (e.g., U.S. Pat. No. 5,994,444). In some embodiments, one or more photolyzable nitric oxide donors 104 may be bound to a polymer. Methods that can be used to couple nitric oxide donors to a polymeric matrix have been reported (e.g., U.S. Pat. No. 5,405,919). In some-embodiments, one or more photolyzable nitric oxide donors 104 may be coupled to polymeric materials used to produce condoms. Accordingly, in some embodiments, one or more photolyzable nitric oxide donors 104 may be coupled to a condom.

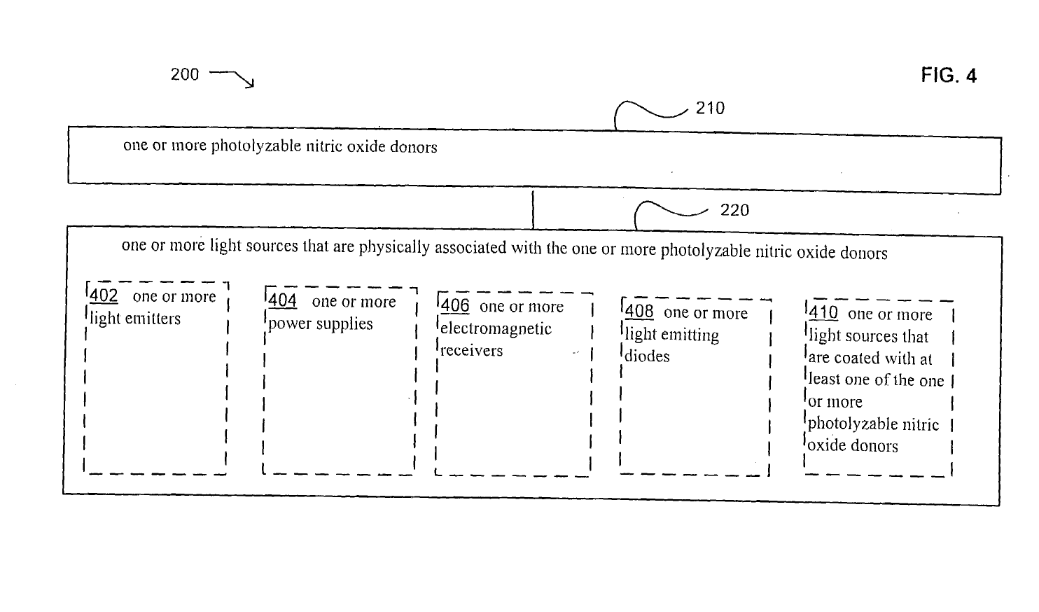

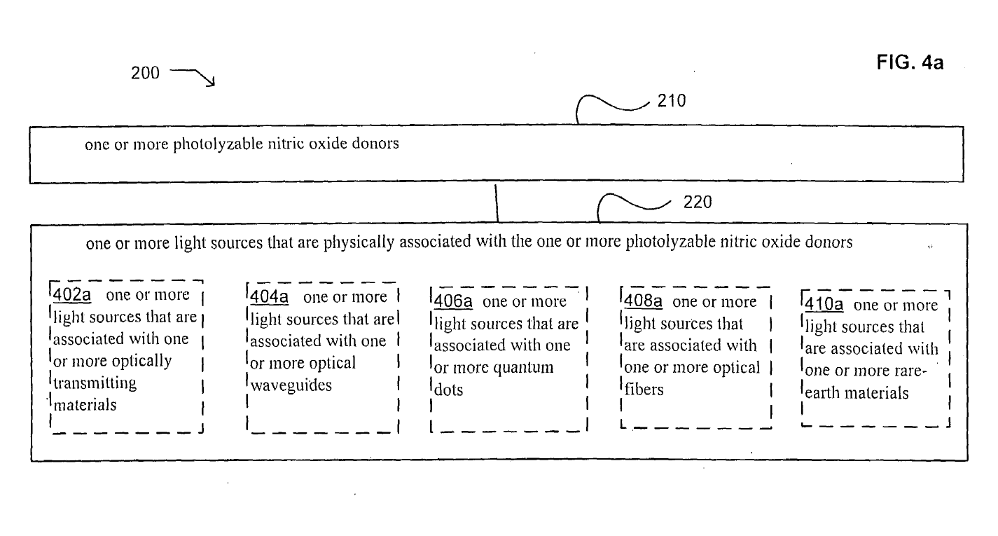

FIG. 4 illustrates alternative embodiments of embodiment 200 of device 102 within system 100 of FIG. 2. FIG. 4 illustrates example embodiments of module 220. Additional embodiments may include an embodiment 402, an embodiment 404, an embodiment 406, an embodiment 408, and/or an embodiment 410.

At embodiment 402, module 220 may include one or more light emitters. In some embodiments, one or more light sources 106 may include one or more light emitters. Numerous types of light emitters may be associated with one or more light sources 106. Examples of such light emitters include, but are not limited to, light emitting diodes, filaments, arc lamps, fluorescent light emitters, phosphorescent light emitters, chemiluminescent emitters, and the like. In some embodiments, one or more light emitters may be coupled with one or more quantum dots. In some embodiments, one or more light emitters may be coupled with one or more rare-earth materials.

At embodiment 404, module 220 may include one or more power supplies. In some embodiments, one or more light sources 106 may include one or more power supplies. Numerous types of power supplies may be associated with one or more light sources 106. Examples of such power supplies include, but are not limited to, batteries (e.g., thin film batteries), electromagnetic receivers 108, line power, and the like.

At embodiment 406, module 220 may include one or more electromagnetic receivers. In some embodiments, one or more light sources 106 may include one or more electromagnetic receivers 108. In some embodiments, one or more electromagnetic receivers 108 may be used to receive electromagnetic energy 110 for use in providing power to one or more light emitters. Methods to construct electromagnetic receivers 108 have been described (e.g., U.S. Pat. No. 5,571,152).

At embodiment 408, module 220 may include one or more light emitting diodes. In some embodiments, one or more light sources 106 may include one or more light emitting diodes. One or more light sources 106 may include one or more light emitting diodes that are configured to emit light of select wavelengths. For example light emitting diodes may be configured to emit infrared light, visible light, near-ultraviolet light, or ultraviolet light. In some embodiments, a light source 106 may include a conventional light emitting diode that can include a variety of inorganic semiconductor materials. Examples of such materials and the emitting light include, but are not limited to, aluminium gallium arsenide (red and infrared), aluminium gallium phosphide (green), aluminium gallium indium phosphide (high-brightness orange-red, orange, yellow, and green), gallium arsenide phosphide (red, orange-red, orange, and yellow), gallium phosphide (red, yellow and green), gallium nitride (green, pure green, emerald green, blue, and white (if it has an AlGaN Quantum Barrier)), indium gallium nitride (near ultraviolet, bluish-green and blue), silicon carbide (blue), silicon (blue), sapphire (blue), zinc selenide (blue), diamond (ultraviolet), aluminium nitride (near to far ultraviolet), aluminium gallium nitride (near to far ultraviolet), aluminium gallium indium nitride (near to far ultraviolet).

At embodiment 410, module 220 may include one or more light sources that are coated with at least one of the one or more photolyzable nitric oxide donors. In some embodiments, one or more light sources 106 may be coated with at least one photolyzable nitric oxide donor 104. For example, in some embodiments, a light source 106 may be configured as a wand that emits light which can be coated with one or more photolyzable nitric oxide donors 104. In some embodiments, a light source 106 may be configured as a sheet that is coated with one or more photolyzable nitric oxide donors 104. In some embodiments, one or more light sources 106 may be partially coated with one or more photolyzable nitric oxide donors 104.

FIG. 4a illustrates alternative embodiments of embodiment 200 of device 102 within system 100 of FIG. 2. FIG. 4a illustrates example embodiments of module 220. Additional embodiments may include an embodiment 402a, an embodiment 404a, an embodiment 406a, an embodiment 408a, and/or an embodiment 410a.

At embodiment 402a, module 220 may include one or more light sources that are associated with one or more optically transmitting materials. In some embodiments, one or more light sources 106 may be associated with one or more optically transmitting materials. In some embodiments, optically transmitting materials include all substances that function to alter or control electromagnetic radiation in the ultraviolet, visible, or infrared spectral regions. Such materials may be fabricated into optical elements such as lenses, mirrors, windows, prisms, polarizers, detectors, and modulators. These materials may refract, reflect, transmit, disperse, polarize, detect, and/or transform light. Examples of optically transmitting materials include, but are not limited to, glass, crystalline materials, polymers, plastics, and the like. In some embodiments, one or more light sources 106 may include fused silica which transmits to about 180 nm. In some embodiments, one or more light sources 106 may include calcium fluoride which transmits into the ultraviolet region to about 140 nm. Accordingly, a light source 106 may include numerous types of optically transmitting materials.

At embodiment 404a, module 220 may include one or more light sources that are associated with one or more optical waveguides. In some embodiments, one or more light sources 106 may be associated with one or more optical waveguides. Numerous types of optical waveguides may be associated with one or more light sources 106. For example, in some embodiments, a waveguide may be an optical fiber waveguide. In some embodiments, a waveguide may be a rectangular waveguide. In some embodiments, a waveguide may be a dielectric slab waveguide. In some embodiments, optical waveguides may include, but are not limited to, planar wave guides, strip waveguides, and/or fiber waveguides. In some embodiments, an optical waveguide may have a single-mode structure. In some embodiments, an optical waveguide may have a multi-mode structure. In some embodiments, an optical waveguide may exhibit a step refractive index distribution. In some embodiments, an optical waveguide may exhibit a gradient refractive index distribution. An optical waveguide may be constructed from numerous types of materials that include, but are not limited to, glass, polymers, semiconductors, and the like. Methods to construct optical waveguides have been described (e.g., U.S. Pat. No. 7,283,710).

At embodiment 406a, module 220 may include one or more light sources that are associated with one or more quantum dots. In some embodiments, one or more light sources 106 may be associated with one or more quantum dots (e.g., U.S. Pat. No. 7,235,361; herein incorporated by reference). For example, in some embodiments, one or more light sources 106 may be configured to emit one or more wavelengths of light that are absorbed by one or more quantum dots. In some embodiments, one or more quantum dots may be configured to absorb light and then emit one or more wavelengths of light that cause release of nitric oxide from one or more nitric oxide donors. Accordingly, in some embodiments, emission from one or more first quantum dots may be tuned to facilitate release of nitric oxide from one or more first photolyzable nitric oxide donors 104 and emission from one or more second quantum dots may be tuned to facilitate release of nitric oxide from one or more second photolyzable nitric oxide donors 104.

At embodiment 408a, module 220 may include one or more light sources that are associated with one or more optical fibers. In some embodiments, one or more photolyzable nitric oxide donors 104 may be associated with one or more optical fibers. In some embodiments, one or more photolyzable nitric oxide donors 104 may be directly associated with one or more optical fibers. For example, in some embodiments, one or more optical fibers may be directly coated with one or more photolyzable nitric oxide donors 104. In some embodiments, one or more optical fibers may be directly coated with one or more compositions that include one or more photolyzable nitric oxide donors 104.

In some embodiments, one or more portions of one or more optical fibers may be directly coated with one or more photolyzable nitric oxide donors 104. In some embodiments, one or more portions of one or more optical fibers may be directly coated with one or more compositions that include one or more photolyzable nitric oxide donors 104. In some embodiments, one or more photolyzable nitric oxide donors 104 may be indirectly associated with one or more optical fibers. For example, in some embodiments, one or more optical fibers may be inserted into a structure that is coated with one or more photolyzable nitric oxide donors 104. In some embodiments, one or more optical fibers may be inserted into a structure that is coated with one or more compositions that include one or more photolyzable nitric oxide donors 104. In some embodiments, one or more optical fibers may be inserted into a structure that is partially coated with one or more photolyzable nitric oxide donors 104. In some embodiments, one or more optical fibers may be inserted into a structure that is partially coated with one or more compositions that include one or more photolyzable nitric oxide donors 104. For example, in some embodiments, one or more optical fibers may be inserted into one or more tubes that are coated with one or more photolyzable nitric oxide donors 104. In some embodiments, one or more optical fibers may be inserted into one or more tubes that are coated with one or more compositions that include one or more photolyzable nitric oxide donors 104. One or more photolyzable nitric oxide donors 104 may be associated with numerous types of optical fibers. Methods to construct optical fibers have been described. Examples of optical fibers include, but are not limited to, optical fibers that include a single core and/or one or more cores. In some embodiments, an optical fiber may include silica glass. In some embodiments, an optical fiber may include a cladding. Optical fibers have been described (e.g., U.S. Pat. Nos. 7,295,741; 7,295,737).

At embodiment 410a, module 220 may include one or more light sources that are associated with one or more rare-earth materials. In some embodiments, one or more light sources 106 may include one or more photolyzable nitric oxide donors 104 that are associated with one or more rare-earth materials. In some embodiments, one or more rare-earth materials may include one or more rare-earth elements. The rare-earth elements are a collection of sixteen chemical elements in the periodic table, namely scandium, yttrium, and fourteen of the fifteen lanthanoids (excluding promethium). In some embodiments, one or more rare-earth materials may include one or more rare-earth elements that fluoresce.

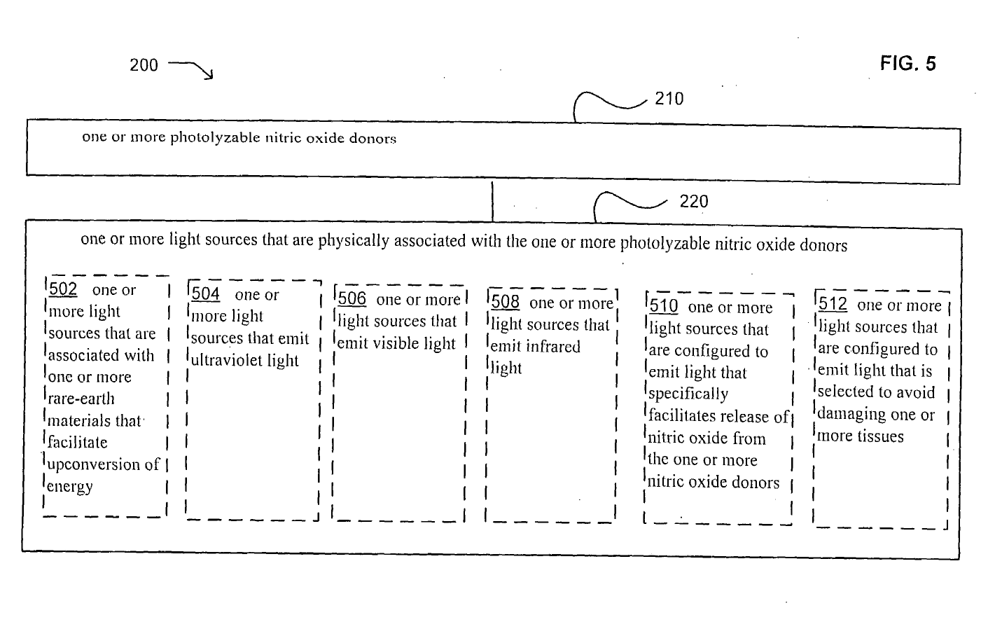

FIG. 5 illustrates alternative embodiments of embodiment 200 of device 102 within system 100 of FIG. 2. FIG. 5 illustrates example embodiments of module 220. Additional embodiments may include an embodiment 502, an embodiment 504, an embodiment 506, an embodiment 508, an embodiment 510, and/or an embodiment 512.

At embodiment 502, module 220 may include one or more light sources that are associated with one or more rare-earth materials that facilitate upconversion of energy. In some embodiments, one or more light sources 106 may be associated with one or more rare-earth materials that facilitate upconversion of energy. In some embodiments, infrared light may be upconverted to visible light (e.g., Mendioroz et al., Optical Materials, 26:351-357 (2004). In some embodiments, infrared light may be upconverted to ultraviolet light (e.g., Mendioroz et al., Optical Materials, 26:351-357 (2004). In some embodiments, one or more light sources 106 may include one or more rare-earth materials (e.g., ytterbium-erbium, ytterbium-thulium, or the like) that facilitate upconversion of energy (e.g., U.S. Pat. No. 7,088,040; herein incorporated by reference). For example, in some embodiments, one or more light sources 106 may be associated with Nd3+ doped KPb2Cl5 crystals. In some embodiments, one or more light sources 106 may be associated with thiogallates doped with rare earths, such as CaGa2S4:Ce3+ and SrGa2S4:Ce3+. In some embodiments, one or more light sources 106 may be associated with aluminates that are doped with rare earths, such as YAlO3:Ce3+, YGaO3:Ce3+, Y(Al,Ga)O3:Ce3+, and orthosilicates M2SiO5:Ce3+(M:Sc, Y, Sc) doped with rare earths, such as, for example, Y2SiO5 :Ce3+. In some embodiments, yttrium may be replaced by scandium or lanthanum (e.g., U.S. Pat. Nos. 6,812,500 and 6,327,074; herein incorporated by reference). Numerous materials that may be used to upconvert energy have been described (e.g., U.S. Pat. Nos. 5,956,172; 5,943,160; 7,235,189; 7,215,687; herein incorporated by reference).

At embodiment 504, module 220 may include one or more light sources that emit ultraviolet light. In some embodiments, one or more light sources 106 may emit ultraviolet light. In some embodiments, one or more light sources 106 may emit a broad spectrum of ultraviolet light. In some embodiments, one or more light sources 106 may emit a narrow spectrum of ultraviolet light. In some embodiments, one or more light sources 106 that emit one or more wavelengths of ultraviolet light that are specifically selected to release nitric oxide from one or more photolyzable nitric oxide donors 104. In some embodiments, one or more light sources 106 may emit ultraviolet light that does not include one or more wavelengths of light. In some embodiments, one or more light sources 106 may emit ultraviolet light that is selected to avoid and/or reduce damage to structures and/or tissues of an individual 126. For example, in some embodiments, one or more light sources 106 may emit ultraviolet light that does not include wavelengths of light that are absorbed by nucleic acids. In some embodiments, one or more light sources 106 may emit ultraviolet light that does not include wavelengths of light that are absorbed by polypeptides. In some embodiments, one or more light sources 106 may emit light that does not include one or more wavelengths of ultraviolet light within the following range: 250-320 nm. For example, in some embodiments, one or more light sources 106 may not emit 260 nm light. In some embodiments, one or more light sources 106 may not emit 280 nm light. In some embodiments, one or more light sources 106 may not emit 260 nm light or 280 nm light. Accordingly, numerous combinations of wavelengths of light may be excluded from emission by one or more light sources 106. In some embodiments, light may be emitted continuously. In some embodiments, light may be emitted as a flash. In some embodiments, light may be emitted alternately as continuous light and a flash. In some embodiments, light may be emitted as a pulse. In some embodiments, light may be emitted continuously, as a flash, as a pulse, or substantially any combination thereof.

At embodiment 506, module 220 may include one or more light sources that emit visible light. In some embodiments, one or more light sources 106 may emit visible light. In some embodiments, one or more light sources 106 may emit a broad spectrum of visible light. In some embodiments, one or more light sources 106 may emit a narrow spectrum of visible light. In some embodiments, one or more light sources 106 may emit one or more wavelengths of visible light that are specifically selected to release nitric oxide from one or more photolyzable nitric oxide donors 104. In some embodiments, one or more light sources 106 may emit visible light that does not include one or more wavelengths of light. In some embodiments, one or more light sources 106 may emit visible light that is selected to avoid and/or reduce damage to structures and/or tissues of an individual 126. Accordingly, numerous combinations of wavelengths of light may be excluded from emission by one or more light sources 106. In some embodiments, light may be emitted continuously. In some embodiments, light may be emitted as a flash. In some embodiments, light may be emitted alternately as continuous light and a flash. In some embodiments, light may be emitted as a pulse. In some embodiments, light may be emitted continuously, as a flash, as a pulse, or substantially any combination thereof. In some embodiments, the visible light may be upconverted.

At embodiment 508, module 220 may include one or more light sources that emit infrared light. In some embodiments, one or more light sources 106 may emit infrared light. In some embodiments, one or more light sources 106 may emit a broad spectrum of infrared light. In some embodiments, one or more light sources 106 may emit a narrow spectrum of infrared light. In some embodiments, one or more light sources 106 may emit one or more wavelengths of infrared light that are specifically selected to release nitric oxide from one or more photolyzable nitric oxide donors 104. In some embodiments, one or more light sources 106 may emit infrared light that does not include one or more wavelengths of light. In some embodiments, one or more light sources 106 may emit infrared light that is selected to avoid and/or reduce damage to structures and/or tissues of an individual 126. Accordingly, numerous combinations of wavelengths of light may be excluded from emission by one or more light sources 106. In some embodiments, light may be emitted continuously. In some embodiments, light may be emitted as a flash. In some embodiments, light may be emitted alternately as continuous light and a flash. In some embodiments, light may be emitted as a pulse. In some embodiments, light may be emitted continuously, as a flash, as a pulse, or substantially any combination thereof. In some embodiments, the infrared light may be upconverted.

At embodiment 510, module 220 may include one or more light sources that are configured to emit light that specifically facilitates release of nitric oxide from the one or more nitric oxide donors. In some embodiments, one or more light sources 106 may emit light that specifically facilitates release of nitric oxide from the one or more nitric oxide donors. For example, in some embodiments, one or more light sources 106 may be configured to emit light that includes one or more wavelengths of light that correspond to the absorption maximum for one or more nitric oxide donors. Examples of nitric oxide donors and their associated λₘₐₓ (nm) are provided in Table I below. Accordingly, one or more light sources 106 maybe configured to emit numerous wavelengths of light.

TABLE I
Example Nitric Oxide Donors

| Compound Name | λₘₐₓ (nm) |
|---|---|
| O²-(Acetoxymethyl) 1-(N,N-Diethylamino)diazen-1-ium-1,2- | 230 |
| diolate | |
| O²-(Acetoxymethyl) 1-(Pyrrolidin-1-yl)diazen-1-ium-1,2- | 256 |
| diolate | |
| Sodium 1-(N-Benzyl-N-methylamino)diazen-1-ium-1,2- | 252 |
| diolate | |
| O²-[(2,3,4,6-Tetra-O-acetyl)-β-D-glucosyl] 1-[4-(2,3- | 232 |
| Dihydroxypropyl)piperazin-1 | |
| Sodium 1-[4-(2,3-Dihydroxypropyl)piperazin-1-yl-]diazen-1- | 248.5 |
| ium-1,2-diolate | |
| O²-Methyl 1-[(4-Carboxamido)piperidin-1-yl]diazen-1-ium- | 241 |
| 1,2-diolate | |
| O²-(2-Chloropyrimidin-4-yl) 1-(Pyrrolidin-1-yl)diazen-1-ium- | 274 |
| 1,2-diolate | |
| O²(2,4-Dinitrophenyl) 1-[4-(N,N-Diethylcarboxamido) | 300 |
| piperazin-1-yl]diazen-1-ium-1,2-diolate | |
| O²-(2,4-Dinitrophenyl) 1-(4-Nicotinylpiperazin-1-yl)diazen- | 300 |
| 1-ium-1,2-diolate | |
| O²-(2,4-Dinitrophenyl) 1-{4-[2-(4-{2- | 300 |
| Methylpropyl}phenyl)propionyl]piperazin-1-yl}diazen-1- | |
| ium-1,2-diolate | |
| Sodium 1-(4-Benzyloxycarbonylpiperazin-1-yl)diazen-1-ium- | 252 |
| 1,2-diolate | |
| O²-(2,4-Dinitrophenyl) 1-[4-(tert-Butoxycarbonyl)piperazin-1- | 299 |
| yl]diazen-1-ium-1,2-diolate | |
| O²-(2,4-Dinitrophenyl) 1-(4-Acetylpiperazin-1-yl)diazen-1- | 394 |
| ium-1,2-diolate | |
| O²-(2,4-Dinitrophenyl) 1-[4-(Succinimidoxycarbonyl) | 300 |
| piperazin-1-yl]diazen-1-ium-1,2-diolate | |
| O²-(2,4-Dinitrophenyl) 1-(Piperazin-1-yl)diazen-1-ium-1,2- | 297 |
| diolate, Hydrochloride Salt | |
| O²-(2,3,4,6-Tetra-O-acetyl-D-glucopyranosyl) 1-(N,N- | 228 |
| Diethylamino)diazen-1-ium-1,2-diolate | |
| O²-(-D-Glucopyranosyl) 1-(N,N-Diethylamino)diazen-1-ium- | 228 |
| 1,2-diolate | |
| Sodium (Z)-1-(N,N-Diethylamino)diazen-1-ium-1,2-diolate | 250 |
| 1-[N-(2-Aminoethyl)-N-(2-ammonioethyl)amino]diazen-1- | 252 |
| ium-1,2-diolate | |
| Sodium 1-(N,N-Dimethylamino)diazen-1-ium-1,2-diolate | 250 |
| O²-(2,4-Dinitrophenyl) 1-(N,N-Diethylamino)diazen-1-ium- | 302 |
| 1,2-diolate | |
| 1-[N-(3-Aminopropyl)-N-(3-ammoniopropyl]diazen-1-ium- | 252 |
| 1,2-diolate | |
| 1-[N-(3-Aminopropyl)-N-(3-ammoniopropyl]diazen-1-ium- | 252 |
| 1,2-diolate | |
| Bis-diazeniumdiolated benzyl imidate dehydrate | 264 |
| p-Bisdiazeniumdiolated benzene | 316 |
| Methane Trisdiazeniumdiolate trihydrate | 316 |
| O²-(β-D-Glucopyranosyl) 1-(Isopropylamino)diazen-1-ium- | 278 |
| 1,2-diolate | |
| Sodium 1-[4-(5-Dimethylamino-1-naphthalenesulfonyl) | 344 |
| piperazin-1-yl]diazen-1-ium-1,2-diolate | |
| 1-(2-Methyl-1-propenyl)piperidine diazeniumdiolate | 246 |
| 1-(2-Methyl-1-propenyl)pyrrolidine diazeniumdiolate | 246 |
| O²-Vinyl 1-(Pyrrolidin-1-yl)diazen-1-ium-1,2-diolate | 268 |
| 1-{N-[3-Aminopropyl]-N-[4-(3-aminopropylammoniobutyl) | 252 |
| ]}diazen-1-ium-1,2-diolate | |
| Disodium 1-[(2-Carboxylato)pyrrolidin-1-yl]diazen-1-ium-1,2- | 252 |
| diolate | |
| 1-[N-(3-Ammoniopropyl)-N-(n-propyl)amino]diazen-1-ium- | 250 |
| 1,2-diolate | |
| (Z)-1-{N-Methyl-N-[6-(N-methylammoniohexyl)amino | 250 |
| ]}diazen-1-ium-1,2-diolate | |
| O²-(2,4-Dinitrophenyl) 1-[(4-Ethoxycarbonyl)piperazin-1- | 300 |
| yl]diazen-1-ium-1,2-diolate | |

At embodiment 512, module 220 may include one or more light sources that are configured to emit light that is selected to avoid damaging one or more tissues. In some embodiments, one or more light sources 106 malt be configured to emit light that is selected to avoid damaging one or more tissues. In some embodiments, one or more light sources 106 may emit light that is selected to avoid and/or reduce damage to structures and/or tissues of an individual 126. For example, in some embodiments, one or more light sources 106 may emit light that does not include wavelengths of light that are absorbed by nucleic acids. In some embodiments, one or more light sources 106 may emit light that does not include wavelengths of light that are absorbed by polypeptides. In some embodiments, one or more light sources 106 may emit light that does not include one or more wavelengths of light within the following range: 250-320 nm. For example, in some embodiments, one or-more light sources 106 may not emit 260 nm light. In some embodiments, one or more light sources 106 may not emit 280 nm light. In some embodiments, one or more light sources 106 may not emit 260 nm light or 280 nm light. Accordingly, numerous combinations of wavelengths of light may be excluded from emission by one or more light sources 106. In some embodiments, light may be emitted continuously. In some embodiments, light may be emitted as a flash. In some embodiments, light may be emitted alternately as continuous light and a flash. In some embodiments, light may be emitted as a pulse.

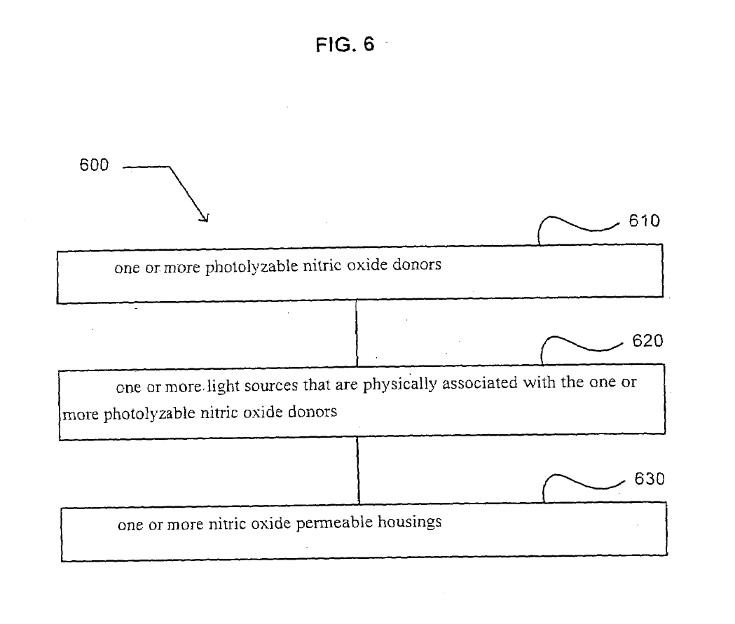

FIG. 6 illustrates alternative embodiment 600 of device 102 within system 100 of FIG. 1. In FIG. 6, discussion and explanation may be provided with respect to the above-described example of FIG. 1, and/or with respect to other examples and contexts. In some embodiments, modules 210 and 220 as described with respect to embodiment 200 of device 102 of FIG. 2 may correspond to modules 610 and 620 as described with respect to embodiment 600 of device 102 within system 100. However, it should be understood that the modules may execute operations in a number of other environments and contexts, and/or modified versions of FIG. 1. Also, although the various modules are presented in the sequence(s) illustrated, it should be understood that the various modules may be configured in numerous orientations.

The embodiment 600 includes module 610 that includes one or more photolyzable nitric oxide donors. In some embodiments, device 102 includes one or more photolyzable nitric oxide donors 104 that release nitric oxide upon photolysis. Examples of such photolyzable nitric oxide donors 104 include, but are not limited to, diazeniumdiolates (e.g., U.S. Pat. Nos. 7,105,502; 7,122,529; 6,673,338; herein incorporated by reference), trans-[RuCl([15]aneN4)NO]+2 (Ferezin et al., Nitric Oxide, 13:170-175 (2005), Bonaventura et al., Nitric Oxide, 10:83-91 (2004)), nitrosyl ligands (e.g., U.S. Pat. No. 5,665,077; herein incorporated by reference, Chmura et al., Nitric Oxide, 15:370-379 (2005), Flitney et al., Br. J. Pharmacol., 107:842-848 (1992), Flitney et al. Br. J. Pharmacol., 117:1549-1557 (1996), Matthews et al., Br. J. Pharmacol., 113:87-94 (1994)), 6-Nitrobenzo[a]pyrene (e.g., Fukuhara et al., J. Am. Chem. Soc., 123:8662-8666 (2001)), S-nitroso-glutathione (e.g., Rotta et al., Braz. J. Med. Res., 36:587-594 (2003), Flitney and Megson, J. Physiol., 550:819-828 (2003)), S-nitrosothiols (e.g., Andrews et al., British Journal of Pharmacology, 138:932-940 (2003), Singh et al., FEBS Lett., 360:47-51 (1995)), 2-Methyl-2-nitrosopropane (e.g., Pou et al., Mol. Pharm., 46:709-715 (1994), Wang et al., Chem. Rev., 102:1091-1134 (2002)), imidazolyl derivatives (e.g., U.S. Pat. No. 5,374,710; herein incorporated by reference).

The embodiment 600 includes module 620 that includes one or more light sources that are physically associated with the one or more photolyzable nitric oxide donors. In some embodiments, device 102 includes one or more light sources 106 that are physically associated with one or more photolyzable nitric oxide donors 104. In some embodiments, the one or more light sources 106 may be directly coupled to one or more photolyzable nitric oxide donors 104. For example, in some embodiments, the one or more photolyzable nitric oxide donors 104 may be chemically coupled to a surface of the light source 106 (e.g., chemically coupled to a polymer coating on the light source). In some embodiments, one or more photolyzable nitric oxide donors 104 may be indirectly coupled to one or more light sources 106. For example, in some embodiments, one or more photolyzable nitric oxide donors 104 may be coupled to a material that is used to coat the one or more light sources 106.

The embodiment 600 includes module 630 that includes one or more nitric oxide permeable housings. In some embodiments, device 102 may include one or more nitric oxide permeable housings 114. In some embodiments, nitric oxide permeable housings 114 may be configured for implantation within an individual 126. In some embodiments, nitric oxide permeable housings 114 may be configured to facilitate application of nitric oxide to a surface. In some embodiments, one or more nitric oxide permeable housings 114 may be configured to facilitate application-of nitric oxide to one or more surfaces of an individual 126. For example, in some embodiments, one or more nitric oxide permeable housings 114 may be configured as a canister having a nitric oxide permeable end that may be positioned on a skin surface of an individual to deliver nitric oxide to the skin surface. In some embodiments, one or more nitric oxide permeable housings 114 may be configured for insertion into the urethra of a male. In some embodiments, one or more nitric oxide permeable housings 114 may be configured for insertion into the vagina of a female.

In some embodiments, one or more nitric oxide permeable housings 114 may be configured to enclose at least a portion of one or more photolyzable nitric oxide donors 104. In some embodiments, one or more nitric oxide permeable housings 114 may be configured to enclose at least a portion of one or more light sources 106. In some embodiments, one or more nitric oxide permeable housings 114 may be configured to enclose at least a portion of one or more control units 116. In some embodiments, one or more nitric oxide permeable housings 114 may be configured to enclose at least a portion of one or more nitric oxide sensors 120. In some embodiments, one or more nitric oxide permeable housings 114 may be configured to enclose one or more photolyzable nitric oxide donors 104. In some embodiments, one or more nitric oxide permeable housings 114 may be configured to enclose one or more light sources 106. In some embodiments, one or more nitric oxide permeable housings 114 may be configured to enclose one or more photolyzable nitric oxide donors 104 and one or more light sources 106. In some embodiments, one or more nitric oxide permeable housings 114 may be configured to enclose one or more photolyzable nitric oxide donors 104, one or more light sources 106, and one or more control units 116. In some embodiments, one or more nitric oxide permeable housings 114 may be configured to enclose one or more photolyzable nitric oxide donors 104, one or more light sources 106, one or more control units 116, and one or more electromagnetic receivers 108. In some embodiments, one or more nitric oxide permeable housings 114 may be configured to enclose one or more photolyzable nitric oxide donors 104, one or more light sources 106, one or more control units 116, one or more electromagnetic receivers 108, or substantially any combination thereof. In some embodiments, one or more nitric oxide permeable housings 114 may be configured to include one or more compartments. For example, in some embodiments, a nitric oxide permeable housing 14 may include a compartment that is configured to accept one or more light sources 106 and a second compartment that is configured to accept one or more photolyzable nitric oxide donors 104. In some embodiments, a nitric oxide permeable housing 114 may include a compartment that is configured to accept one or more light sources 106, a light permeable divider, and a second compartment that is configured to accept one or more photolyzable nitric oxide donors 104. In some embodiments, a light permeable divider may be made of a material that allows light to pass through the divider. Examples of such material include, but are not limited to, plastic, quartz, and the like.

Nitric oxide permeable housings 114 may be constructed of numerous types of materials and combinations of materials. Examples of such materials include, but are not limited to, ceramics, polymeric materials, metals, plastics, and the like. In some embodiments, nitric oxide permeable housings 114 may include numerous combinations of materials.

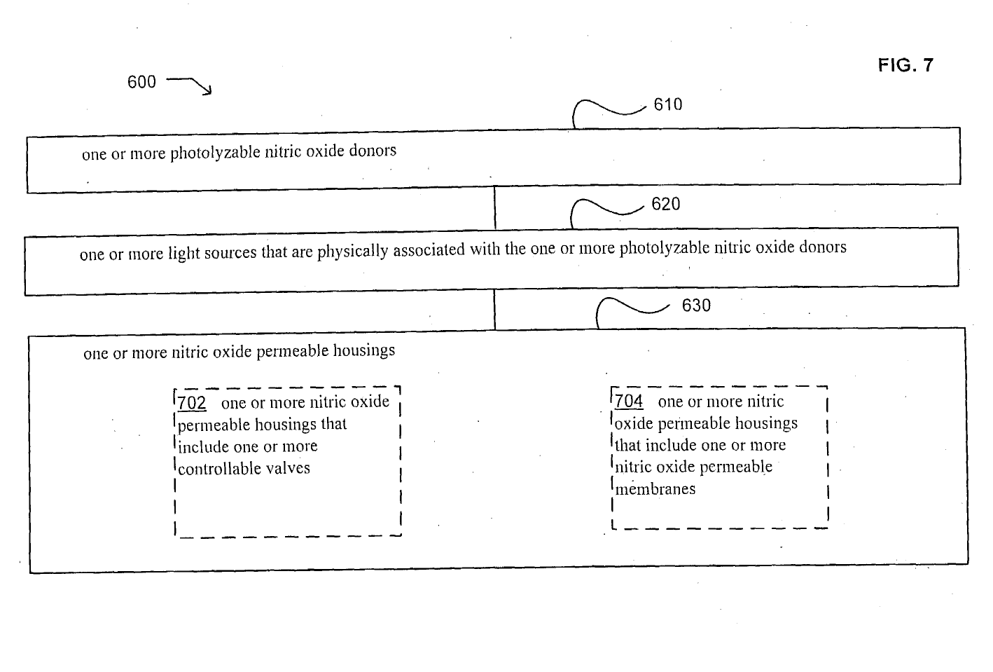

FIG. 7 illustrates alternative embodiments of embodiment 600 of device 102 within system 100 of FIG. 6. FIG. 7 illustrates example embodiments of module 630. Additional embodiments may include an embodiment 702, and/or an embodiment 704.

At embodiment 702, module 630 may include one or more nitric oxide permeable housings that include one or more controllable valves. In some embodiments, one or more nitric oxide permeable housings 114 may include one or more controllable valves. In some embodiments, a controllable valve may provide for the passage of nitric oxide. In some embodiments, a controllable valve may provide for the passage of one or more photolyzable nitric oxide donors 104. In some embodiments, one or more controllable valves may include one or more electromagnets that provide for controlled opening and closing of an orifice associated with the valve. In some embodiments, one or more controllable valves may include one or more screw closures that provide for controlled opening and closing of the valve. For example, in some embodiments, a nitric oxide permeable housing 114 may include an electric motor that operates the screw mechanism to provide for opening and closure of an orifice associated with the nitric oxide permeable housing 114. Numerous controllable valves may be associated with one or more nitric oxide permeable housings 114. In some embodiments, a nitric oxide permeable housing 114 may include one or more valves that are controllable by one or more control units 116. Accordingly, in some embodiments, valves may be opened or closed in response to one or more nitric oxide sensors 120, one or more signals 118, one or more information packets, one or more management units 122, or substantially any combination thereof.

At embodiment 704, module 630 may include one or more nitric oxide permeable housings that include one or more nitric oxide permeable membranes. In some embodiments, one or more nitric oxide permeable housings 114 may include one or more nitric oxide permeable membranes. For example, in some embodiments, a nitric oxide permeable housing 114 may include a nitric oxide impermeable metal canister that is coupled to a nitric oxide permeable membrane (e.g., U.S. Patent Application No.: 20020026937). In some embodiments, a nitric oxide permeable housing 114 may include a selectively permeable membrane. For example, in some embodiments, a nitric oxide permeable housing 114 may include a selectively permeable, hydrophilic polyester co-polymer membrane system that includes a copolymer with 70% polyester and 30% polyether (e.g., Sympatex™ 10 μm membrane, see Hardwick et al., Clinical Science, 100:395-400 (2001)). In some embodiments, a nitric oxide permeable housing 114 may include a scintered glass portion that is permeable to nitric oxide. Accordingly, nitric oxide permeable housings 114 may include numerous types of porous ceramics that are permeable to nitric oxide. In some embodiments, a nitric oxide permeable housing 114 may include a nitric oxide permeable coating (e.g., U.S. Patent Application Nos.: 20050220838; 20030093143).

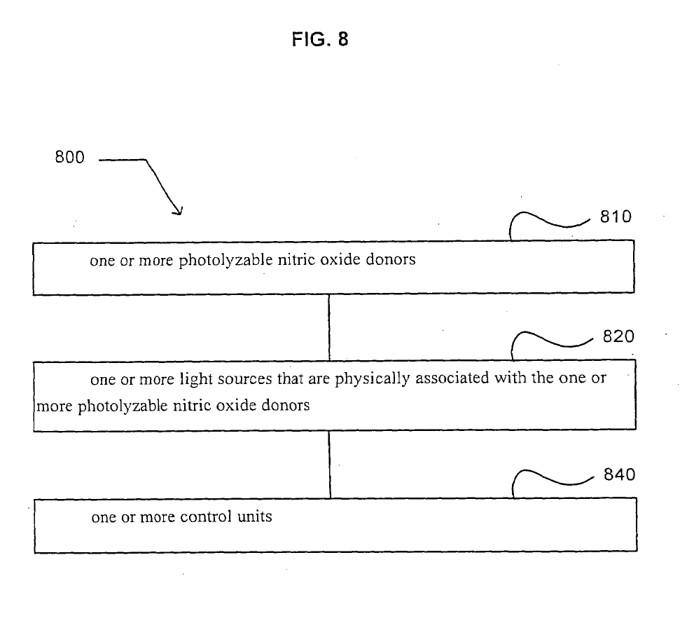

FIG. 8 illustrates alternative embodiment 800 of device 102 within system 100 of FIG. 1. In FIG. 8, discussion and explanation may be provided with respect to the above-described example of FIG. 1, and/or with respect to other examples and contexts. In some embodiments, modules 210 and 220 as described with respect to embodiment 200 of device 102 of FIG. 2 may correspond to modules 810 and 820 as described with respect to embodiment 800 of FIG. 8. However, it should be understood that the modules may execute operations in a number of other environments and contexts, and/or modified versions of FIG. 1. Also, although the various modules are presented in the sequence(s) illustrated, it should be understood that the various modules may be configured in numerous orientations.

The embodiment 800 includes module 810 that includes one or more photolyzable nitric oxide donors. In some embodiments, a device 102 includes one or more photolyzable nitric oxide donors 104 that release nitric oxide upon photolysis. Examples of such photolyzable nitric oxide donors 104 include, but are not limited to, diazeniumdiolates (e.g., U.S. Pat. Nos. 7,105,502; 7,122,529; 6,673,338; herein incorporated by reference), trans-[RuCl([15]aneN4)NO]+2 (Ferezin et al., Nitric Oxide, 13:170-175 (2005), Bonaventura et al., Nitric Oxide, 10:83-91 (2004)), nitrosyl ligands (e.g., U.S. Pat. No. 5,665,077; herein incorporated by reference, Chmura et al., Nitric Oxide, 15:370-379 (2005), Flitney et al., Br. J. Pharmacol., 107:842-848 (1992), Flitney et al., Br. J. Pharmacol., 117:1549-1557 (1996), Matthews et al., Br. J. Pharmacol., 113:87-94 (1994)), 6-Nitrobenzo[a]pyrene (e.g., Fukuhara et al., J. Am. Chem. Soc., 123:8662-8666 (2001)), S-nitroso-glutathione (e.g., Rotta et al., Braz. J. Med. Res., 36:587-594 (2003), Flitney and Megson, J. Physiol., 550:819-828 (2003)), S-nitrosothiols (e.g., Andrews et al., British Journal of Pharmacology, 138:932-940 (2003), Singh et al., FEBS Lett., 360:47-51 (1995)), 2-Methyl-2-nitrosopropane (e.g., Pou et al., Mol. Pharm., 46:709-715 (1994), Wang et al., Chem. Rev., 102:1091-1134 (2002)), imidazolyl derivatives (e.g., U.S. Pat. No. 5,374,710; herein incorporated by reference).

The embodiment 800 includes module 820 that includes one or more light sources that are physically associated width the one or more photolyzable nitric oxide donors. In some embodiments, device 102 includes one or more light sources 106 that are physically associated with one or more photolyzable nitric oxide donors 104. In some embodiments, the one or more light sources 106 may be directly coupled to one or more photolyzable nitric oxide donors 104. For example, in some embodiments, the one or more photolyzable nitric oxide donors 104 may be chemically coupled to a surface of the light source 106 (e.g., chemically coupled to a polymer coating on the light source). In some embodiments, one or more photolyzable nitric oxide donors 104 may be indirectly coupled to one or more light sources 106. For example, in some embodiments, one or more photolyzable nitric oxide donors 104 may be coupled to a material that is used to coat the one or more light sources 106.

The embodiment 800 includes module 840 that includes one or more control units. In some embodiments, device 102 may include one or more control units 116. A device 102 may include numerous types of control units 116. In some embodiments, one or more control units 116 may be operably coupled with one or more light sources 106, one or more nitric oxide sensors 120, one or more electromagnetic receivers 108, one or more electromagnetic transmitters 112, or substantially any combination thereof. In some embodiments, one or more control units 116 may be operably coupled to other components t-rough use of one or more wireless connections, one or more hardwired connections, or substantially any combination thereof. Control units 116 may be configured in numerous ways. For example, in some embodiments, a control unit 116 may be configured as an on/off switch. Accordingly, in some embodiments, a control unit 116 may be configured to turn a light source 106 on and/or off. In some embodiments, a control unit 116 may be configured to control the emission of light from one or more light sources 106. For example, in some embodiments, one or more control units 116 may regulate the intensity of light emitted from one or more light sources 106, the duration of light emitted from one or more light sources 106, the frequency of light emitted from one or more light sources 106, wavelengths of light emitted from one or more light sources 106, or substantially any combination thereof. In some embodiments, one or more control units 116 may be configured to receive one or more signals 118 from one or more nitric oxide sensors 120. Accordingly, in some embodiments, one or more control units 116 may be configured to control one or more light sources 106 in response to one or more signals 118 received from one or more nitric oxide sensors 120. For example, in some embodiments, one or more nitric oxide sensors 120 may sense a low concentration of nitric oxide in one or more tissues and send one or more signals 118 to one or more control units 116. The one or more control units 116 may then turn one or more light sources 106 on to facilitate release of nitric oxide from one or more photolyzable nitric oxide donors 104. Accordingly, in some embodiments, one or more nitric oxide sensors 120 may sense a high concentration of nitric oxide in one or more tissues and send one or more signals 118 to one or more control units 116. The one or more control units 116 may then turn one or more light sources 106 off to end release of nitric oxide from one or more photolyzable nitric oxide donors 104. In some embodiments, one or more control units 116 may be programmed to control one or more light sources 106. For example, in some embodiments, one or more control units 116 may be programmed to turn one or more light sources 106 on for a predetermined amount of time and then turn off. Accordingly, in some embodiments, one or more control units 116 may be preprogrammed. In some embodiments, one or more control units 116 may be dynamically programmed. For example, in some embodiments, one or more management units 122 may receive one or more signals 118 from one or more nitric oxide sensors 120 and program one or more control units 116 in response to the one or more signals 118 received from the one or more nitric oxide sensors 120. In some embodiments, one or more control units 116 may include one or more receivers that are able to receive one or more signals 118, one or more information packets, or substantially any combination thereof. Control units 116 may be configured in numerous ways. For example, in some embodiments, one or more control units I 16 may be operably coupled to one or more light sources 106 that include numerous light emitting diodes that emit light of different wavelengths. Accordingly, in some embodiments, one or more control units 116 may control the wavelengths of light emitted by the one or more light sources 106 by controlling the operation of light emitting diodes that emit light of the selected wavelength. Accordingly, control units 116 may be configured in numerous ways and utilize numerous types of mechanisms.

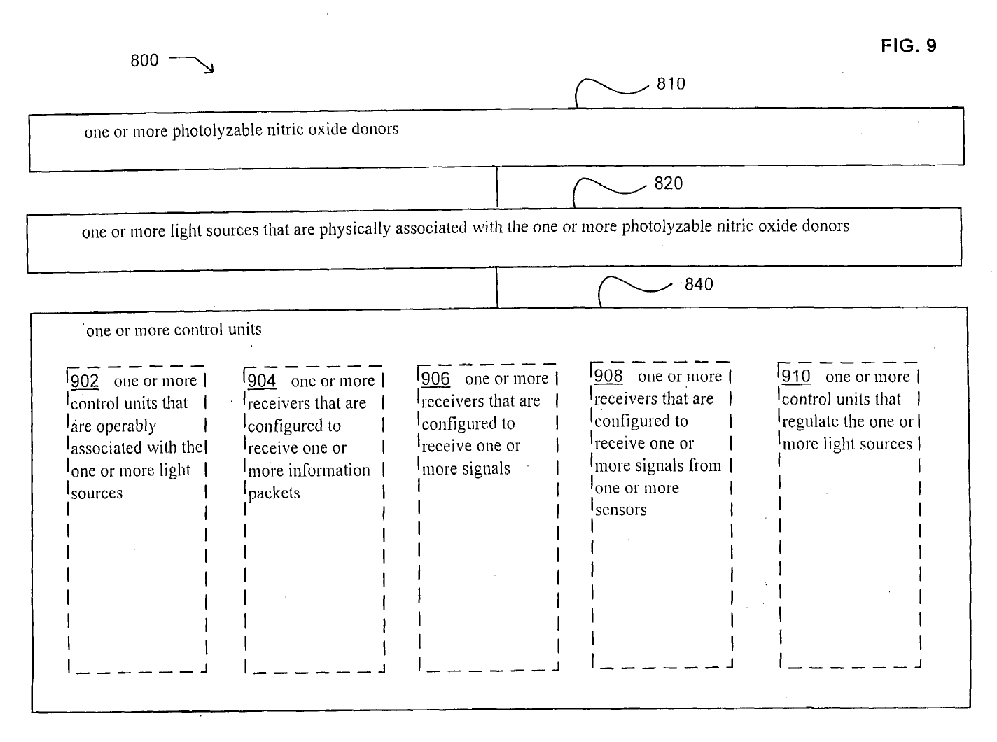

FIG. 9 illustrates alternative embodiments of embodiment 800 of device 102 within system 100 of FIG. 8. FIG. 9 illustrates example embodiments of module 840. Additional embodiments may include an embodiment 902, an embodiment 904, an embodiment 906, an embodiment 908, and/or an embodiment 910.

At embodiment 902, module 840 may include one or more control units that are operably associated with the one or more light sources. In some embodiments, one or more control units 116 may be operably associated with one or more light sources 106. In some embodiments, the one or more control units 116 may be operably associated width one or more light sources 106 through use of a hardwired connection. In some embodiments, the one or more control units 116 may be operably associated with one or more light sources 106 through use of a wireless connection. In some embodiments, one or more control units 116 may include numerous types of receivers. Examples of such receivers include, but are not limited to, receivers that receive one or more optical signals 118, radio signals 118, wireless signals 118, hardwired signals 118, infrared signals 118, ultrasonic signals 118, and the like. Such receivers are known and have been described (e.g., U.S. Pat. Nos. RE39,785; 7,218,900; 7,254,160; 7,245,894; 7,206,605; herein incorporated by reference).

At embodiment 904, module 840 may include one or more receivers that are configured to receive one or more information packets. In some embodiments, one or more control units 116 may include one or more receivers that are configured to receive one or more information packets. In some embodiments, one or more control units 116 may be configured to receive one or more information packets that include numerous types of information. Examples of such information include, but are not limited to, intensity of light to be emitted by one or more light sources 106, duration of light to be emitted by one or more light sources 106, frequency of light to be emitted by one or more light sources 106, wavelengths of light to be emitted by one or more light sources 106, and the like.

At embodiment 906, module 840 may include one or more receivers that are configured to receive one or more signals. In some embodiments, one or more control units 116 may include one or more receivers that are configured to receive one or more signals 118. A control unit 116 may include a receiver that is configured to receive numerous types of signals 118. Examples of-such signals 118 include, but are not limited to, optical signals 118, radio signals 118, wireless signals 118, hardwired signals 118, infrared signals 118, ultrasonic signals 118, and the like. In some embodiments, one or more signals 118 may not be encrypted. In some embodiments, one or more signals 118 may be encrypted. In some embodiments, one or more signals 118 may be sent through use of a secure mode of transmission. In some embodiments, one or more signals 118 may be coded for receipt by a specific individual 126. In some embodiments, such code may include anonymous code that is specific for an individual 126. Accordingly, information included within one or more signals 118 may be protected against being accessed by others who are not the intended recipient.

At embodiment 908, module 840 may include one or more receivers that are configured to receive one or more signals from one or more sensors. In some embodiments, one or more control units 116 may include one or more receivers that are configured to receive one or more signals 118 from one or more sensors. In some embodiments, one or more control units 116 may include one or more receivers that are configured to receive one or more signals 118 from one or more nitric oxide sensors 120. Control units 116 may be configured to receive one or more signals 118 from numerous types of sensor. Examples of such sensors include, but are not limited to, temperature sensors, blood pressure sensors, pulse rate sensors, hydrostatic pressure sensors, clocks, and the like.

At embodiment 910, module 840 may include one or more control units that regulate the one or more light sources. In some embodiments, one or more control units 116 may include one or more control units 116 that regulate one or more light sources 106. One or more control units 116 may regulate numerous aspects of one or more light sources 106. Examples of such aspects include, but are not limited to, intensity of emitted light, duration of emitted light, pulse frequency of emitted light, wavelengths of emitted light, and the like.

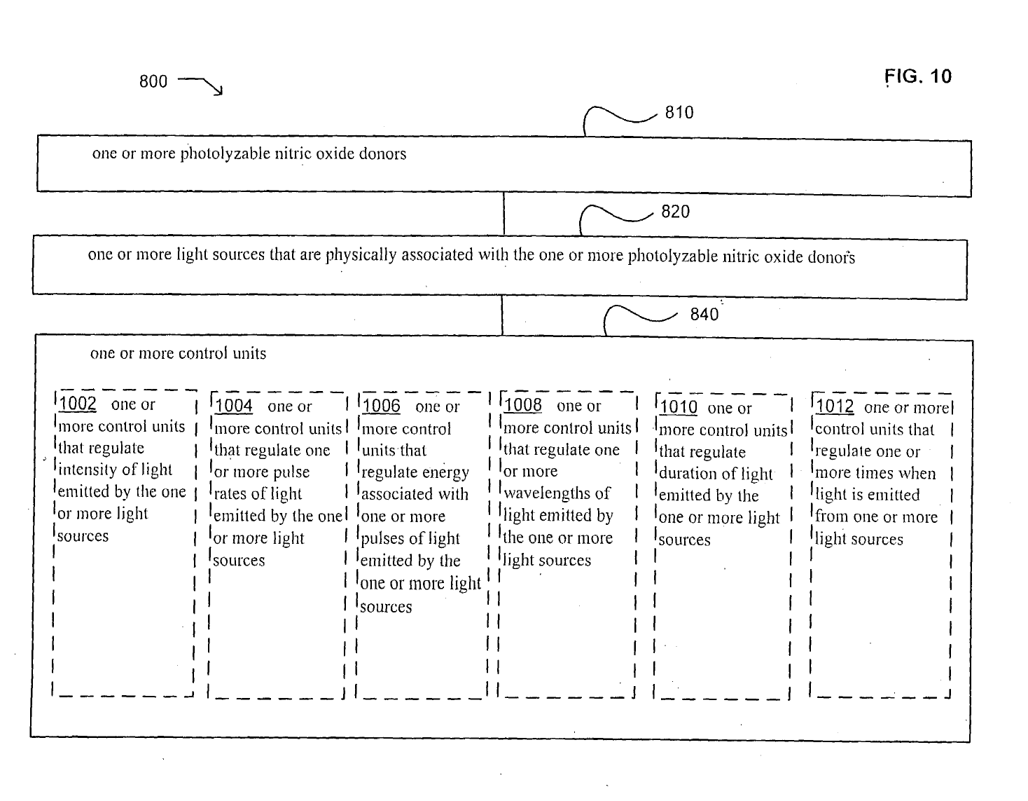

FIG. 10 illustrates alternative embodiments of embodiment 800 device 102 of FIG. 8. FIG. 10 illustrates example embodiments of module 840. Additional embodiments may include an embodiment 1002, an embodiment 1004, an embodiment 1006, an embodiment 1008, an embodiment 1010, and/or an embodiment 1012.

At embodiment 1002, module 840 may include one or more control units that regulate intensity of light emitted by the one or more light sources. In some embodiments, one or more control units 116 may include one or more control units 116 that regulate the intensity of light emitted by one or more light sources 106. For example, in some embodiments, one or more control units 116 may regulate the current flowing through a light source 106 to regulate the intensity of light emitted from the light source. For example, in some embodiments, one or more control units 116 may include a potentiometer.

At embodiment 1004, module 840 may include one or more control units that regulate one or more pulse rates of light emitted by the one or more light sources. In some embodiments, one or more control units 116 may include one or more control units 116 that regulate one or more pulse rates of light emitted by the one or more light sources 106. For example, in some embodiments, one or more control units 116 may cause a light source 106 to emit light in short pulses (e.g., nanosecond pulses, microsecond pulses). In some embodiments, one or more control units 116 may cause a light source 106 to emit light in medium pulses (e.g., second pulses, minute pulses). In some embodiments, one or more control units 16 may cause a light source 106 to emit light in medium pulses (e.g., hour pulses, day long pulses).

At embodiment 1006, module 840 may include one or more control units that regulate energy associated with one or more pulses of light emitted by the one or more light sources. In some embodiments, one or more control units 116 may include one or more control units 116 that regulate energy associated with one or more pulses of light emitted by the one or more light sources 106. For example, in some embodiments, one or more control units 116 may regulate the current flowing through a light source 106 to regulate the energy associated with one or more pulses of light emitted by the one or more light sources 106. In some embodiments, one or more control units 116 may regulate what wavelengths of light are emitted by a light source 106 to regulate the energy associated with one or more pulses of light emitted by the one or more light sources 106.

At embodiment 1008, module 840 may include one or more control units that regulate one or more wavelengths of light emitted by the one or more light sources. In some embodiments, one or more control units 116 may include one or more control units 116 that regulate one or more wavelengths of light emitted by one or more light sources 106. For example, in some embodiments, one or more control units 116 may be coupled to a light source 106 that includes numerous light emitting diodes that emit light of different wavelengths. Accordingly, in some embodiments, one or more control units 116 may regulate wavelengths of light emitted from the light source 106 by selectively illuminating light emitting diodes that emit the desired wavelengths of light.

At embodiment 1010, module 840 may include one or more control units that regulate duration of light emitted by the one or more light sources. In some embodiments, one or more control units 116 may include one or more control units 116 that regulate the duration of light emitted by one or more light sources 106. For example, one or more control units 116 may cause one or more light sources 106 to emit light for a period of nanoseconds, microseconds, milliseconds, seconds, minutes, hours, days, and the like.

At embodiment 1012, module 840 may include one or more control units that regulate one or more times when light is emitted from one or more light sources. In some embodiments, one or more control units 116 may include one or more control units 116 that regulate one or more times when light is emitted from one or more light sources 106. For example, in some embodiments, one or more control units 116 may facilitate illumination of one or more photolyzable nitric oxide donors 104 at predetermined time intervals. In some embodiments, one or more control units 116 may facilitate illumination of one or more photolyzable nitric oxide donors 104 at predetermined time intervals. In some embodiments, one or more control units 116 may facilitate illumination of one or more photolyzable nitric oxide donors 104 at selected times during the day. Accordingly, one or more control units may regulate one or more times when one or more light sources emit light.

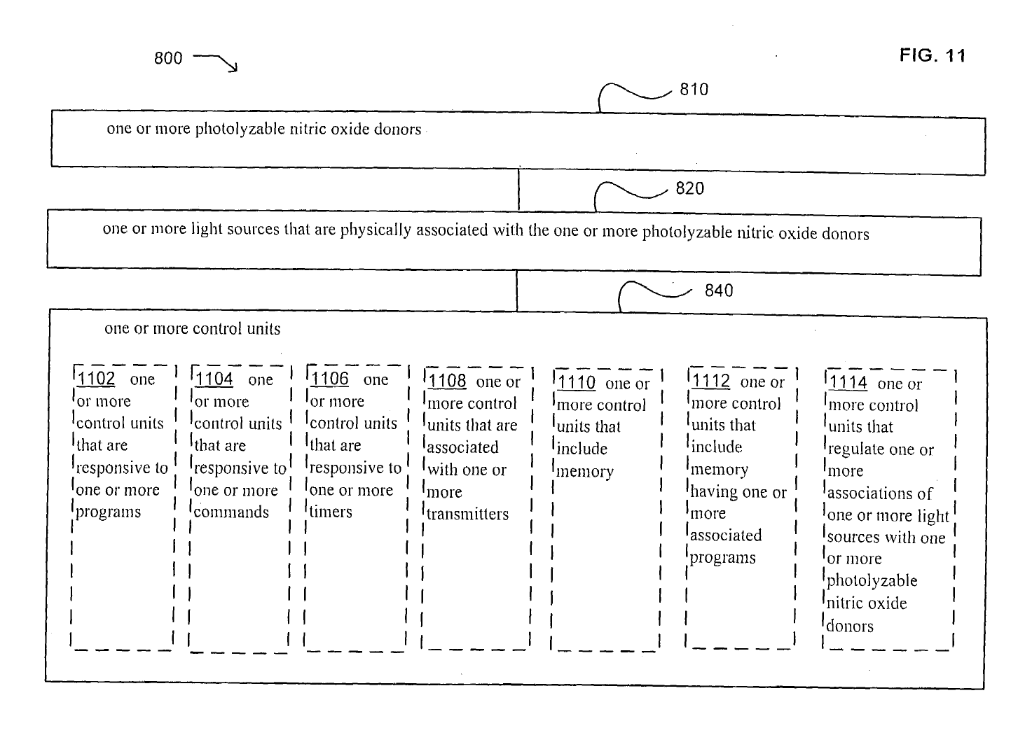

FIG. 11 illustrates alternative embodiments of embodiment 840 of device 102 of FIG. 8. FIG. 11 illustrates example embodiments of module 840. Additional embodiments may include an embodiment 1102, an embodiment 1104, an embodiment 1106, an embodiment 1108, an embodiment 1110, an embodiment 1112, and/or an embodiment 1114.

At embodiment 1102, module 840 may include one or more control units that are responsive to one or more programs. In some embodiments, one or more control units 116 may include one or more control units 116 that are responsive to one or more programs. For example, in some embodiments, one or more control units 116 may be responsive to a programmed set of instructions. In some embodiments, the one or more control units 116 may be directly programmed. For example, in some embodiments, one or more control units 116 may include a programmable memory that can include instructions. In some embodiments, the one or more control units 116 may receive instructions from a program that is associated with one or more management units 122.

At embodiment 1104, module 840 may include one or more control units that are responsive to one or more commands. In some embodiments, one or more control units 116 may include one or more control units 116 that are responsive to one or more commands. For example, in some embodiments, one or more control units 116 may receive one or more signals 118 that act as commands for the one or more control units 116. In some embodiments, one or more control units 116 may receive one or more information packets that act as commands for the one or more control units 116.

At embodiment 1106, module 840 may include one or more control units that are responsive to one or more timers. In some embodiments, one or more control units 116 may include one or more control units 116 that are responsive to one or more timers. In some embodiments, one or more control units 116 may be configured to include one or more timers to which the one or more control units 116 are responsive. In some embodiments, one or more control units 116 may be responsive to one or more timers that are remote from the one or more control units 116. For example, in some embodiments, one or more control units 116 may be responsive to one or more timers that are associated with one or more management units 122 that send instructions to the one or more control units 116.

At embodiment 1108, nodule 840 may include one or more control units that are associated with one or more transmitters. In some embodiments, one or more control units 116 may be associated with one or more transmitters. In some embodiments, one or more control units 116 may transmit one or more signals 118. In some embodiments, one or more control units 116 may transmit one or more information packets. Accordingly, in some embodiments, control units 116 may be configured to operate within a feedback scheme that can receive information and transmit information to regulate the generation of nitric oxide. For example, in some embodiments, one or more control units 116 may regulate one or more light sources 106 to generate nitric oxide and then transmit information related to the operation of the one or more light sources 106. In some embodiments, one or more control units 116 may regulate one or more nitric oxide permeable housings 114 to release nitric oxide and then transmit information related to the operation of the one or more housings.

At embodiment 1110, module 840 may include one or more control units that include memory. In some embodiments, one or more control units 116 may include memory. Numerous types of memory may be associated with one or more control units 116. Examples of such memory include, but are not limited to, magnetic memory, semiconductor memory, and the like.

At embodiment 1112, module 840 may include one or more control units that include memory having one or more associated programs. In some embodiments, one or more control units 116 may include memory having one or more associated programs. In some embodiments, one or more control units 116 may include memory that includes a program that provides instructions for operating one or more light sources 106. For example, in some embodiments, one or more control units 116 may receive information with regard to a current concentration of nitric oxide within an area and then process the information with one or more programs to determine one or more operating parameters for one or more light sources 106. In some embodiments, one or more control units 116 may receive information with regard to bacterial contamination within an area and then process the information with one or more programs to determine one or more operating parameters for one or more light sources 106. Accordingly, one or more control units 116 may include one or more programs that may be configured to respond to numerous types of information.

At embodiment 1114, module 840 may include one or more control units that regulate one or more associations of one or more light sources with one or more photolyzable nitric oxide donors. In some embodiments, one or more control units 116 may regulate one or more associations of one or more light sources 106 with one or more photolyzable nitric oxide donors 104. For example, in some embodiments, one or more control units 116 may regulate one or more connections that couple one or more light sources 106 with one or more optical fibers that are associated with one or more photolyzable nitric oxide donors 104. Accordingly, in some embodiments, one or more control units 116 may regulate light emission through regulation of the coupling of one or more light sources 106 with one or more optically transmitting materials that are associated with one or more photolyzable nitric oxide donors 104.

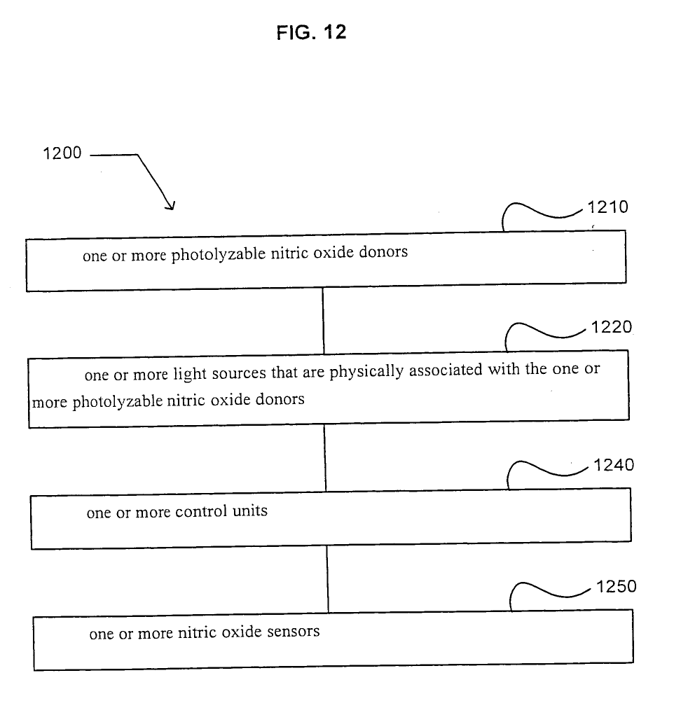

FIG. 1 illustrates alternative embodiments of device 102 within system 100 of FIG. 1. In FIG. 12, discussion and explanation may be provided with respect to the above-described example of FIG. 1, and/or with respect to other examples and contexts. In some embodiments, modules 810, 820, and 840 as described with respect to embodiment 800 of device 102 of FIG. 8 may correspond to modules 1210, 1220, and 1240 as described with respect to embodiment 1200 of device 102 of FIG. 12. However, it should be understood that the modules may execute operations in a number of other environments and contexts, and/or modified versions of FIG. 1. Also, although the various modules are presented in the sequence(s) illustrated, it should be understood that the various modules may be configured in numerous orientations.

The embodiment 1200 includes module 1210 that includes one or more photolyzable nitric oxide donors. In some embodiments, a device 102 includes one or more photolyzable nitric oxide donors 104 that release nitric oxide upon photolysis. Examples of such photolyzable nitric oxide donors 104 include, but are not limited to, diazeniumdiolates (e.g., U.S. Pat. Nos. 7,105,502; 7,122,529; 6,673,338; herein incorporated by reference), trans-[RuCl([15]aneN4)NO]+2 (Ferezin et al., Nitric Oxide, 13:170-175 (2005), Bonaventura et al., Nitric Oxide, 10:83-91 (2004)), nitrosyl ligands (e.g., U.S. Pat. No. 5,665,077; herein incorporated by reference, Chmura et al., Nitric Oxide, 15:370-379 (2005), Flitney et al., Br. J. Pharmacol., 107:842-848 (1992), Flitney et al., Br. J. Pharmacol., 117:1549-1557 (1996), Matthews et al., Br. J. Pharmacol., 113:87-94 (1994)), 6-Nitrobenzo[a]pyrene (e.g., Fukuhara et al., J. Am. Chem. Soc., 123:8662-8666 (2001)), S-nitroso-glutathione (e.g., Rotta et al., Braz. J. Med. Res., 36:587-594 (2003), Flitney and Megson, J. Physiol., 550:819-828 (2003)), S-nitrosothiols (e.g., Andrews et al., British Journal of Pharmacology, 138:932-940 (2003), Singh et al., FEBS Lett., 360:47-51 (1995)), 2-Methyl-2-nitrosopropane (e.g., Pou et al., Mol. Pharm., 46:709-715 (1994), Wang et al., Chem. Rev., 102:1091-1134 (2002)), imidazolyl derivatives (e.g., U.S. Pat. No. 5,374,710; herein incorporated by reference).

The embodiment 1200 includes module 1220 that includes one or more light sources that are physically associated with the one or more photolyzable nitric oxide donors. In some embodiments, device 102 includes one or more light sources 106 that are physically associated with one or more photolyzable nitric oxide donors 104. In some embodiments, the one or more light sources 106 may be directly coupled to one or more photolyzable nitric oxide donors 104. For example, in some embodiments, the one or more photolyzable nitric oxide donors 104 may be chemically coupled to a surface of the light source 106 (e.g., chemically coupled to a polymer coating on the light source). In some embodiments, one or more photolyzable nitric oxide donors 104 may be indirectly coupled to one or more light sources 106. For example, in some embodiments, one or more photolyzable nitric oxide donors 104 may be coupled to a material that is used to coat the one or more light sources 106.

The embodiment 1200 includes module 1240 that includes one or more control units. In some embodiments, device 102 includes one or more control units 116. A device 102 may include numerous types of control units 116. In some embodiments, one or more control units 116 nay be operably coupled with one or more light sources 106, one or more nitric oxide sensors 120, one or more electromagnetic receivers 108, one or more electromagnetic transmitters 112, or substantially any combination thereof. In some embodiments, one or more control units 116 may be operably coupled to other components through use of one or more wireless connections, one or more hardwired connections, or substantially any combination thereof. Control units 116 may be configured in numerous ways. For example, in some embodiments, a control unit 116 may be configured as an on/off switch. Accordingly, in some embodiments, a control unit 116 may be configured to turn a light source 106 on and/or off. In some embodiments, a control unit 116 may be configured to control the emission of light from one or more light sources 106. For example, in some embodiments, one or more control units 116 may regulate the intensity of light emitted from one or more light sources 106, the duration of light emitted from one or more light sources 106, the frequency of light emitted from one or more light sources 106, wavelengths of light emitted from one or more light sources 106, or substantially any combination thereof. In some embodiments, one or more control units 116 may be configured to receive one or more signals 118 from one or more nitric oxide sensors 120. Accordingly, in some embodiments, one or more control units 116 may be configured to control one or more light sources 106 in response to one or more signals 118 received from one or more nitric oxide sensors 120. For example, in some embodiments, one or more nitric oxide sensors 120 may sense a low concentration of nitric oxide in one or more tissues and send one or more signals 118 to one or more control units 116. The one or more control units 116 may then turn one or more light sources 106 on to facilitate release of nitric oxide from one or more photolyzable nitric oxide donors 104. Accordingly, in some embodiments, one or more nitric oxide sensors 120 may sense a high concentration of nitric oxide in one or more tissues and send one or more signals 118 to one or more control units 116. The one or more control units 116 may then turn one or more light sources 106 off to end release of nitric oxide from one or more photolyzable nitric oxide donors 104. In some embodiments, one or more control units 16 may be programmed to control one or more light sources 106. For example, in some embodiments, one or more control units 116 may be programmed to turn one or more light sources 106 on for a predetermined amount of time and then turn off. Accordingly, in some embodiments, one or more control units 116 may be preprogrammed. In some embodiments, one or more control units 116 may be dynamically programmed. For example, in some embodiments, one or more management units 122 may receive one or more signals 118 from one or more nitric oxide sensors 120 and program one or more control units 116 in response to the one or more signals 118 received from the one or more nitric oxide sensors 120. In some embodiments, one or more control units 116 may include one or more receivers that are able to receive one or more signals 118, one or more information packets, or substantially any combination thereof. Control units 116 may be configured in numerous ways. For example, in some embodiments, one or more control units 116 may be operably coupled to one or more light sources 106 that include numerous light emitting diodes that emit light of different wavelengths. Accordingly, in some embodiments, one or more control units 116 may control the wavelengths of light emitted by the one or more light sources 106 by controlling the operation of light emitting diodes that emit light of the selected wavelength. Accordingly, control units 116 may be configured in numerous ways and utilize numerous types of mechanisms.

The embodiment 1200 includes module 1250 that includes one or more nitric oxide sensors. In some embodiments, device 102 includes one or more nitric oxide sensors 120. In some embodiments, one or more nitric oxide sensors 120 may be used to determine the presence of nitric oxide in one or more tissues. In some embodiments, a nitric oxide sensor 120 may be configured for use on the outside surface of an individual 126. For example, in some embodiments, one or more nitric oxide sensors 120 may be configured to detect the concentration of nitric oxide on the surface of skin, a wound, a surface of a table, and the like. In some embodiments, one or more nitric oxide sensors 120 may be configured to be included within one or more housings. In some embodiments, one or more nitric oxide sensors 120 may be configured to be included within one or more nitric oxide permeable housings 114. In some embodiments, a nitric oxide sensor 120 may be configured to utilize fluorescence to detect nitric oxide. For example, in some embodiments, a nitric oxide sensor may detect nitric oxide through use of one or more fluorescent probes, such as 4,5-diaminofluorescein diacetate (EMD Chemicals Inc., San Diego, Calif.). In some embodiments, a nitric oxide sensor may detect nitric oxide through use of one or more electrodes. For example, in some embodiments, a nitric oxide sensor may utilize an electrode that includes a single walled carbon nanotube and an ionic liquid to detect nitric oxide (e.g., Li et al., Electroanalysis, 18:713-718 (2006)). Numerous nitric oxide sensors 120 are commercially available and have been described (e.g., World Precision Instruments, Inc., Sarasota, Fla., USA; U.S. Pat. Nos. 6,100,096; 6,280,604; 5,980,705). In some embodiments, a nitric oxide sensor 120 may include one or more transmitters. In some embodiments, a nitric oxide sensor 120 may include one or more receivers. In some embodiments, a nitric oxide sensor 120 may be configured to transmit one or more signals 118. In some embodiments, a nitric oxide sensor 120 may be configured to receive one or more signals 118.

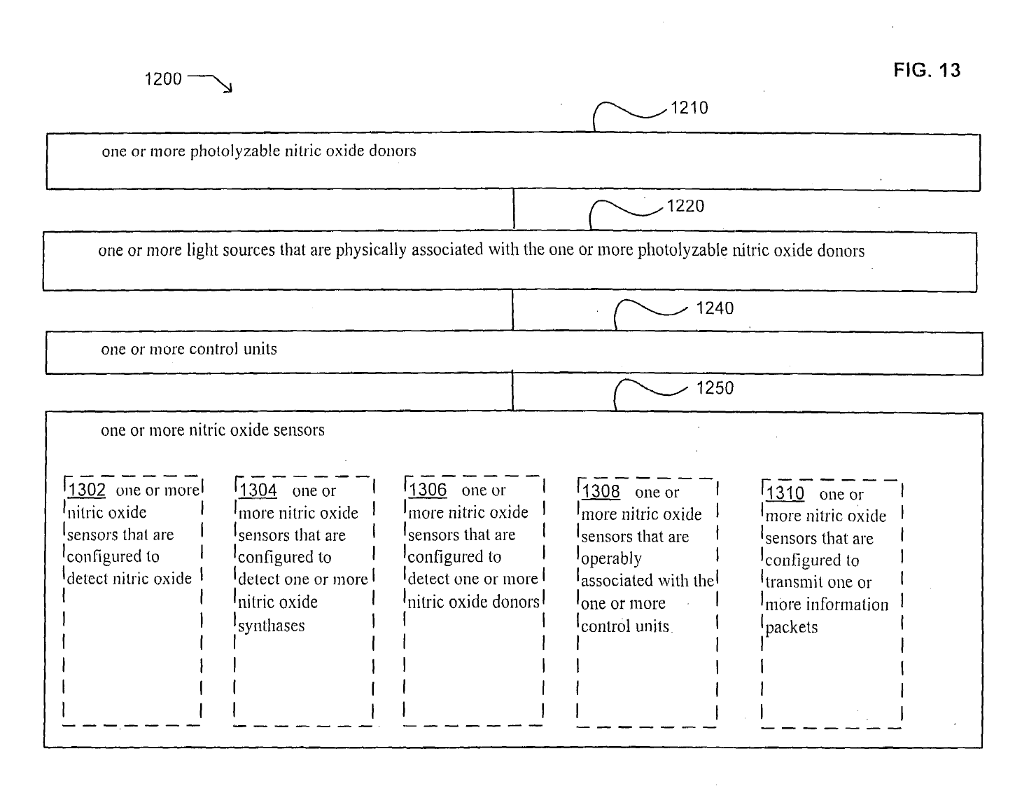

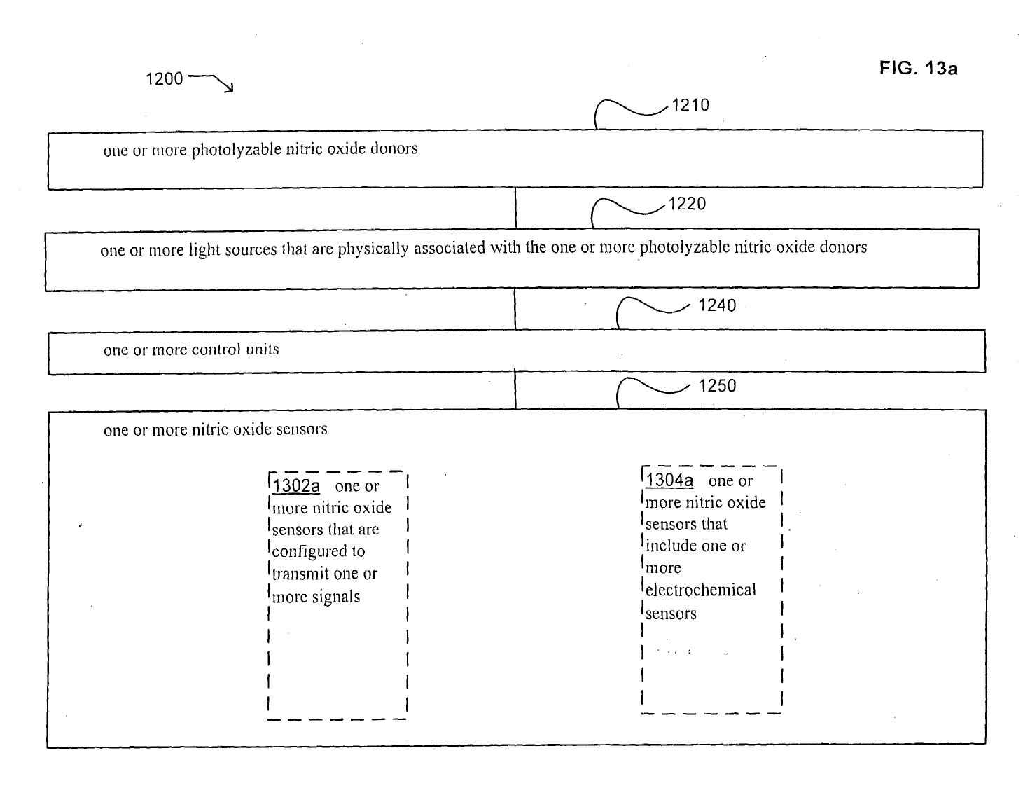

FIG. 13 illustrates alternative embodiments of embodiment 1200 of device 102 within system 100 of FIG. 12. FIG. 13 illustrates example embodiments of module 1250. Additional embodiments may include an embodiment 1302, an embodiment 1304, an embodiment 1306, an embodiment 1308, and/or an embodiment 1310.

At embodiment 1302, module 1250 may include one or more nitric oxide sensors that are configured to detect nitric oxide. In some embodiments, one or more nitric oxide sensors 120 may include one or more nitric oxide sensors 120 that are configured to detect nitric oxide. Nitric oxide sensors 120 may be configured in numerous ways. In some embodiments, a nitric oxide sensor 120 may be configured to utilize fluorescence to detect nitric oxide. For example, in some embodiments, a nitric oxide sensor may detect nitric oxide through use of one or more fluorescent probes, such as 4,5-diaminofluorescein diacetate (EMD Chemicals Inc., San Diego, Calif.). In some embodiments, a nitric oxide sensor may detect nitric oxide through use of one or more electrodes. For example, in some embodiments, a nitric oxide sensor may utilize an electrode that includes a single walled carbon nanotube and an ionic liquid to detect nitric oxide (e.g., Li et al., Electroanalysis, 18:713-718 (2006)). Numerous nitric oxide sensors 120 are commercially available and have been described (e.g., World Precision Instruments, Inc., Sarasota, Fla., USA; U.S. Pat. Nos. 6,100,096; 6,280,604; 5,980,705).

At embodiment 1304, module 1250 may include one or more nitric oxide sensors that are configured to detect one or more nitric oxide synthases. In some embodiments, one or more nitric oxide sensors 120 may include one or more nitric oxide sensors 120 that are configured to detect one or more nitric oxide synthases. In some embodiments, one or more nitric oxide sensors 120 may be configured to detect nitric oxide synthase activity. Nitric oxide synthase detection kits are commercially available (e.g., Cell Technology, Inc., Mountain View, Calif.). In some embodiments, one or more nitric oxide sensors 120 may be configured to detect nitric oxide synthase messenger ribonucleic acid (mRNA). Methods that may be used to detect such mRNA have been reported (e.g., Sonoki et al., Leukemia, 13:713-718 (1999)). In some embodiments, one or more nitric oxide sensors 120 may be configured to detect nitric oxide synthase through immunological methods. Methods that may be used to detect nitric oxide synthase directly been reported (e.g., Burrell et al., J. Histochem. Cytochem., 44:339-346 (1996) and Hattenbach et al., Ophthalmologica, 216:909-214 (2002)). In some embodiments, microelectromechanical systems may be used to detect nitric oxide synthase. In some embodiments, antibodies and/or aptamers that bind to nitric oxide synthase may be used within one or more microelectromechanical systems to detect nitric oxide synthase. Methods to construct microelectromechanical detectors have been described (e.g., Gau et al., Biosensors & Bioelectronics, 16:745-755 (2001)). Accordingly, nitric oxide sensors may be configured in numerous ways to detect one or more nitric oxide synthases.

At embodiment 1306, module 1250 may include one or more nitric oxide sensors that are configured to detect one or more nitric oxide donors. In some embodiments, one or more nitric oxide sensors 120 mall include one or more nitric oxide sensors 120 that are configured to detect one or more nitric oxide donors. In some embodiments, one or more nitric oxide sensors 120 may include one or more surface plasmon resonance chemical electrodes that are configured to detect one or more nitric oxide donors. For example, in some embodiments, one or more nitric oxide sensors 120 may include one or more surface plasmon resonance chemical electrodes that include antibodies and/or aptamers that bind to one or more nitric oxide donors. Accordingly, such electrodes may be used to detect the one or more nitric oxide donors through use of surface plasmon resonance. Methods to construct surface plasmon resonance chemical electrodes are known and have been described (e.g., U.S. Pat. No. 5,858,799; Lin et al., Applied Optics, 46:800-806 (2007)). In some embodiments, antibodies and/or aptamers that bind to one or more nitric oxide donors may be used within one or more microelectromechanical systems to detect one or more nitric oxide donors. Methods to construct microelectromechanical detectors have been described (e.g., Gau et al., Biosensors & Bioelectronics, 16:745-755 (2001)).

At embodiment 1308, module 1250 may include one or more nitric oxide sensors that are operably associated with the one or more control units. In some embodiments, one or more nitric oxide sensors 120 may be operably associated with one or more control units 116. In some embodiments, one or more nitric oxide sensors 120 may be operably associated with one or more control units 116 through a hardwired connection. In some embodiments, one or more nitric oxide sensors 120 may be operably associated with one or more control units 116 through a wireless connection. In some embodiments, one or more nitric oxide sensors 120 may be configured to send one or more signals 118 to one or more control units 116. In some embodiments, one or more nitric oxide sensors 120 may be configured to receive one or more signals 118 from one or more control units 116.

At embodiment 1310, module 1250 may include one or more nitric oxide sensors that are configured to transmit one or more information packets. In some embodiments, one or more nitric oxide sensors 120 may be configured to transmit one or more information packets. In some embodiments, one or more nitric oxide sensors 120 may be configured to transmit one or more information packets to one or more control units 116. Information packets may include numerous types of information. Examples of such information include, but are not limited to, nitric oxide concentration, temperature, time, and the like.

FIG. 13a illustrates alternative embodiments of embodiment 1200 of device 102 within system 100 of FIG. 12. FIG. 13a illustrates example embodiments of module 1250. Additional embodiments may include an embodiment 1302a, and/or an embodiment 1304a.

At embodiment 1302a, module 1250 may include one or more nitric oxide sensors that are configured to transmit one or more signals. In some embodiments, one or more nitric oxide sensors 120 may be configured to transmit one or more signals 118. In some embodiments, one or more nitric oxide sensors 120 may be configured to transmit one or more signals 118. Numerous types of signals 118 may be transmitted. Examples of such signals 118 include, but are not limited to, optical signals 118, radio signals 118, wireless signals 118, hardwired signals 118, infrared signals 118, ultrasonic signals 118, and the like.

At embodiment 1304a, module 1250 may include one or more nitric oxide sensors that include one or more electrochemical sensors. In some embodiments, a device 102 may include one or more nitric oxide sensors 120 that include one or more electrochemical sensors. Nitric oxide sensors 120 may include numerous types of electrochemical sensors. For example, in some embodiments, an electrochemical sensor may be configured as a nitric oxide specific electrode. In some embodiments, a nitric oxide specific electrode may include ruthenium and/or at least one oxide of ruthenium. Methods to construct such electrodes are known and have been described (e.g., U.S. Pat. Nos. 6,280,604; 5,980,705). In some embodiments, a nitric oxide sensor 120 may include an amperometric sensor that includes a sensing electrode that is configured to oxidize nitric oxide complexes to generate an electrical current that indicates the concentration of nitric oxide. Methods to construct such electrodes are known and have been described (e.g., U.S. Patent Application No.: 20070181444 and Ikeda et al., Sensors, 5:161-170 (2005)). Numerous types of electrochemical sensors maybe associated with one or more nitric oxide sensors 120 (e.g., Li et al., Electroanalysis, 18:713-718 (2006)). Electrodes that may be used to detect nitric oxide are commercially available (World Precision Instruments, Sarasota, Fla.). In some embodiments, such electrodes may be used to detect nitric oxide at concentrations of about 0.5 nanomolar and above, and may be about 100 micrometers in diameter (World Precision Instruments, Sarasota, Fla.).

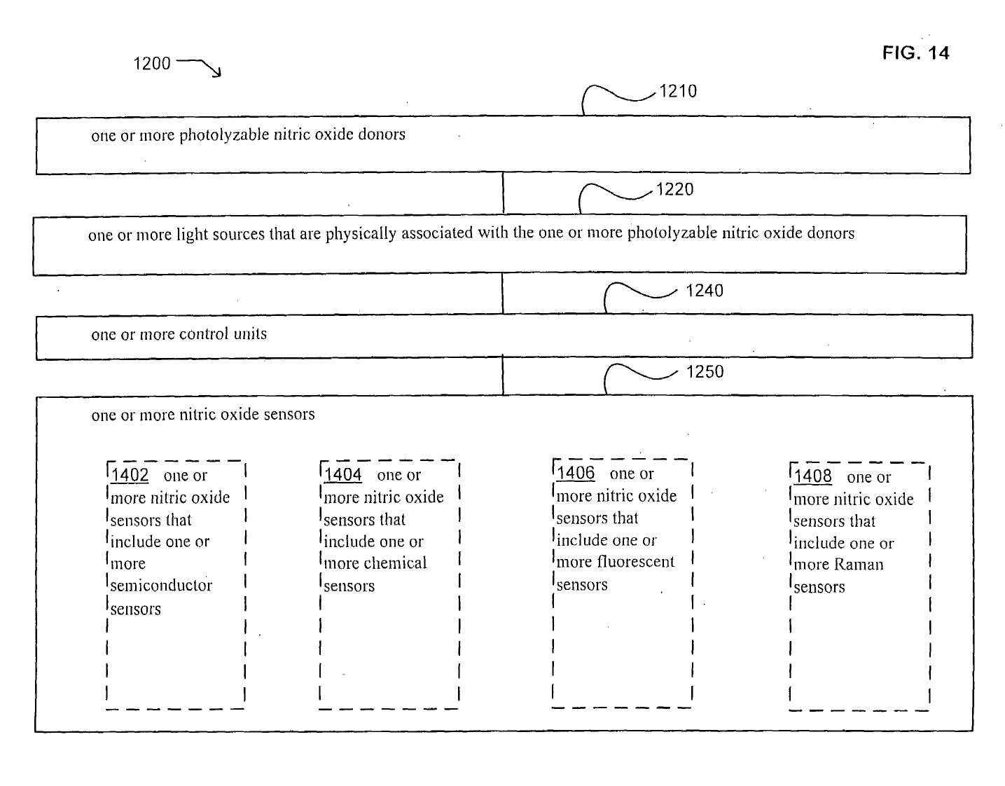

FIG. 14 illustrates alternative embodiments of embodiment 1200 of device 102 within system 100 of FIG. 12. FIG. 14 illustrates example embodiments of module 1250. Additional embodiments may include an embodiment 1402, an embodiment 1404, an embodiment 1406, and/or an embodiment 1408.

At embodiment 1402, module 1250 may include one or more nitric oxide sensors that include one or more semiconductor sensors. In some embodiments, one or more nitric oxide sensors 120 may include one or more nitric oxide sensors 120 that include one or more semiconductor sensors. In some embodiments, the sensor may be a molecular controlled semiconductor resistor of a multilayered GaAs structure to which a layer of multifunctional NO-binding molecules are adsorbed. Such nitric oxide binding molecules may include, but are not limited to, vicinal diamines, metalloporphyrins, metallophthalocyanines, and iron-dithiocarbamate complexes that contain at least one functional group selected from carboxyl, thiol, acyclic sulfide, cyclic disulfide, hydroxamic acid, trichlorosilane or phosphate (e.g., U.S. Published Patent Application No.: 20040072360). In some embodiments, a semiconductive nitric oxide sensor 120 may employ a polycrystalline-oxide semiconductor material that is coated with porous metal electrodes to form a semiconductor sandwich. In some embodiments, the semiconductor material may be formed of SnO₂ or ZnO. The porous electrodes may be formed with platinum and used to measure the conductivity of the semiconductor material. The conductivity of the semiconductor material changes when nitric oxide is absorbed on the surface of the semiconductor material (e.g., U.S. Pat. No. 5,580,433; International Application Publication Number WO 02/057738). Numerous other semiconductor sensors may be used to detect nitric oxide.

At embodiment 1404, module 1250 may include one or more nitric oxide sensors that include one or more chemical sensors. In some embodiments, a device 102 may include one or more nitric oxide sensors 120 that include one or more chemical sensors. For example, in some embodiments, one or more nitric oxide sensors 120 may include one or more chemical sensors that include a reagent solution that undergoes a chemiluminescent reaction with nitric oxide. Accordingly, one or more photodetectors may be used to detect nitric oxide. Methods to construct such detectors are known and have been described (e.g., U.S. Pat. No. 6,100,096). In some embodiments, ozone may be reacted with nitric oxide to produce light in proportion to the amount of nitric oxide present. The light produced may be measured with a photodetector. In some embodiments, sensors may include one or more charge-coupled devices to detect photonic emission.

At embodiment 1406, module 1250 may include one or more nitric oxide sensors that include one or more fluorescent sensors. In some embodiments, one or more nitric oxide sensors 120 may include one or more nitric oxide sensors 120 that include one or more fluorescent sensors. In some embodiments, a fluorescent sensor may include one or more fluorescent probes that may be used to detect nitric oxide. For example, in some embodiments, 4,5-diaminofluorescein may be used to determine nitric oxide concentration (e.g., Rathel et al., Biol. Proced. Online, 5:136-142 (2003)). Probes that may be used to detect nitric oxide are commercially available (EMD Chemicals Inc., San Diego, Calif.).

At embodiment 1408, module 1250 may include one or more nitric oxide sensors that include one or more Raman sensors. In some embodiments, one or more nitric oxide sensors 120 may include one or more nitric oxide sensors 120 that include one or more Raman sensors. Methods to use Raman spectroscopy to detect nitric oxide are known and have been described (e.g., U.S. Patent Application No.: 20060074282). In addition, Raman spectrometers are commercially available (e.g., Raman Systems, Austin, Tex. and B&W Tek, Inc., Newark, Del.).

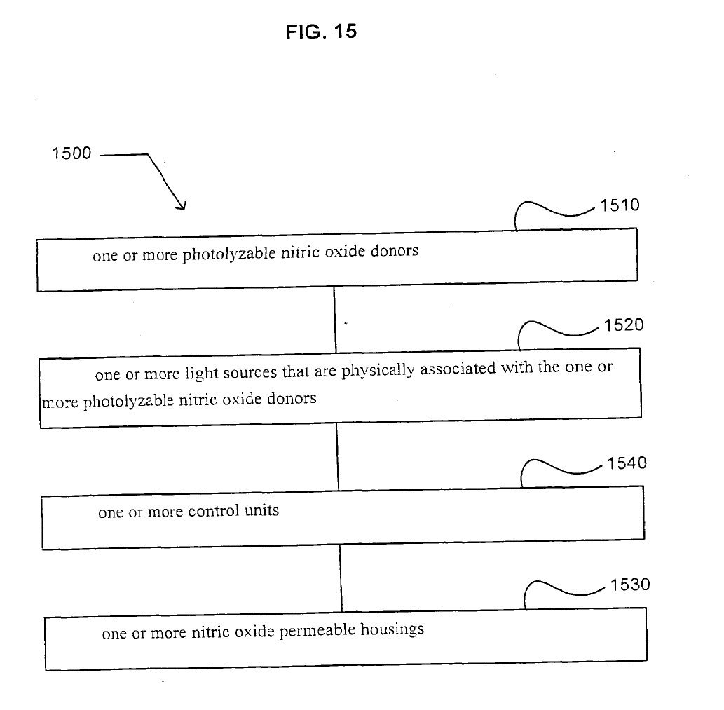

FIG. 15 illustrates alternative embodiment 1500 of device 102 within system 100 of FIG. 1. In FIG. 15, discussion and explanation may be provided with respect to the above-described example of FIG. 1, and/or with respect to other examples and contexts. In some embodiments, modules 810, 820, and 840 as described with respect to embodiment 800 of device 102 of FIG. 8 may correspond to modules 1510, 1520, and 1540 as described with respect to embodiment 1500 of device 102 of FIG. 15. However, it should be understood that the modules may execute operations in a number of other environments and contexts, and/or modified versions of FIG. 1. Also, although the various modules are presented in the sequence(s) illustrated, it should be understood that the various modules mail be configured in numerous orientations.

The embodiment 15500 includes module 510 that includes one or more photolyzable nitric oxide donors. In some embodiments, a device 102 includes one or more photolyzable nitric oxide donors 104 that release nitric oxide upon photolysis. Examples of such photolyzable nitric oxide donors 104 include, but are not limited to, diazeniumdiolates (e.g., U.S. Pat. Nos. 7,105,502; 7,122,529; 6,673,338; herein incorporated by reference), trans-[RuCl([15]aneN4)NO]+2 (Ferezin et al., Nitric Oxide, 13:170-175 (2005), Bonaventura et al., Nitric Oxide, 10:83-91 (2004)), nitrosyl ligands (e.g., U.S. Pat. No. 5,665,077; herein incorporated by reference, Chmura et al., Nitric Oxide, 15:370-379 (2005), Flitney et al., Br. J. Pharmacol., 107:842-848 (1992), Flitney et al., Br. J. Pharmacol., 117:1549-1557 (1996), Matthews et al., Br. J. Pharmacol., 113:87-94 (1994)), 6-Nitrobenzo[a]pyrene (e.g., Fukuhara et al., J. Am. Chem. Soc., 123:8662-8666 (2001)), S-nitroso-glutathione (e.g., Rotta et al., Braz. J. Med. Res., 36:587-594 (2003), Flitney and Megson, J. Physiol., 550:819-828 (2003)), S-nitrosothiols (e.g., Andrews et al., British Journal of Pharmacology, 138:932-940 (2003), Singh et al., FEBS Lett., 360:47-51 (1995)), 2-Methyl-2-nitrosopropane (e.g., Pou et al., Mol. Pharm., 46:709-715 (1994), Wang et al., Chem. Rev., 102:1091-1134 (2002)), imidazolyl derivatives (e.g., U.S. Pat. No. 5,374,710; herein incorporated by reference).

The embodiment 1500 includes module 1520 that includes one or more light sources that are physically associated with the one or more photolyzable nitric oxide donors. In some embodiments, device 102 includes one or more light sources 106 that are physically associated with one or more photolyzable nitric oxide donors 104. In some embodiments, the one or more light sources 106 may be directly coupled to one or more photolyzable nitric oxide donors 104. For example, in some embodiments, the one or more photolyzable nitric oxide donors 104 may be chemically coupled to a surface of the light source 106 (e.g., chemically coupled to a polymer coating on the light source). In some embodiments, one or more photolyzable nitric oxide donors 104 may be indirectly coupled to one or more light sources 106. For example, in some embodiments, one or more photolyzable nitric oxide donors 104 may be coupled to a material that is used to coat the one or more light sources 106.

The embodiment 1500 includes module 1540 that includes one or more control units. In some embodiments device 102 includes one or more control units 116. A device 102 may include numerous types of control units 116. In some embodiments, one or more control units 116 mail be operably coupled with one or more light sources 106, one or more nitric oxide sensors 120, one or more electromagnetic receivers 108, one or more electromagnetic transmitters 112, or substantially any combination thereof. In some embodiments, one or more control units 116 may be operably coupled to other components through use of one or more wireless connections, one or more hardwired connections, or substantially any combination thereof. Control units 116 may be configured in numerous ways. For example, in some embodiments, a control unit 116 may be configured as an on/off switch. Accordingly, in some embodiments, a control unit 116 may be configured to turn a light source 106 on and/or off. In some embodiments, a control unit 116 may be configured to control the emission of light from one or more light sources 106. For example, in some embodiments, one or more control units 116 may regulate the intensity of light emitted from one or more light sources 106, the duration of light emitted from one or more light sources 106, the frequency of light emitted from one or more light sources 106, wavelengths of light emitted from one or more light sources 106, or substantially any combination thereof. In some embodiments, one or more control units 116 may be configured to receive one or more signals 118 from one or more nitric oxide sensors 120. Accordingly, in some embodiments, one or more control units 116 may be configured to control one or more light sources 106 in response to one or more signals 118 received from one or more nitric oxide sensors 120. For example, in some embodiments, one or more nitric oxide sensors 120 may sense a low concentration of nitric oxide in one or more tissues and send one or more signals 118 to one or more control units 116. The one or more control units 116 may then turn one or more light sources 106 on to facilitate release of nitric oxide from one or more photolyzable nitric oxide donors 104. Accordingly, in some embodiments, one or more nitric oxide sensors 120 may sense a high concentration of nitric oxide in one or more tissues and send one or more signals 118 to one or more control units 116. The one or more control units 116 may then turn one or more light sources 106 off to end release of nitric oxide from one or more photolyzable nitric oxide donors 104. In some embodiments, one or more control units 16 may be programmed to control one or more light sources 106. For example, in some embodiments, one or more control units 116 may be programmed to turn one or more light sources 106 on for a predetermined amount of time and then turn off. Accordingly, in some embodiments, one or more control units 116 may be preprogrammed. In some embodiments, one or more control units 116 may be dynamically programmed. For example, in some embodiments, one or more management units 122 may receive one or more signals 118 from one or more nitric oxide sensors 120 and program one or more control units 116 in response to the one or more signals 118 received from the one or more nitric oxide sensors 120. In some embodiments, one or more control units 116 may include one or more receivers that are able to receive one or more signals 118, one or more information packets, or substantially any combination thereof. Control units 116 may be configured in numerous ways. For example, in some embodiments, one or more control units 116 may be operably coupled to one or more light sources 106 that include numerous light emitting diodes that emit light of different wavelengths. Accordingly, in some embodiments, one or more control units 116 may control the wavelengths of light emitted by the one or more light sources 106 by controlling the operation of light emitting diodes that emit light of the selected wavelength. Accordingly, control units 116 may be configured in numerous ways and utilize numerous tripes of mechanisms.

The embodiment 1500 includes module 1530 that includes one or more nitric oxide permeable housings. In some embodiments, device 102 may include one or more nitric oxide permeable housings 114. In some embodiments, nitric oxide permeable housings 114 may be configured for implantation within an individual 126. In some embodiments, nitric oxide permeable housings 114 may be configured to facilitate application of nitric oxide to a surface. In some embodiments, one or more nitric oxide permeable housings 114 may be configured to facilitate application of nitric oxide to one or more surfaces of an individual 126. For example, in some embodiments, one or more nitric oxide permeable housings 114 may be configured as a canister having a nitric oxide permeable end that may be positioned on a skin surface of an individual 126 to deliver nitric oxide to the skin surface. In some embodiments, one or more nitric oxide permeable housings 114 may be configured for insertion into the urethra of a male. In some embodiments, one or more nitric oxide permeable housings 114 may be configured for insertion into the vagina of a female.

In some embodiments, one or more nitric oxide permeable housings 114 may be configured to enclose at least a portion of one or more photolyzable nitric oxide donors 104. In some embodiments, one or more nitric oxide permeable housings 114 may be configured to enclose at least a portion of one or more light sources 106. In some embodiments, one or more nitric oxide permeable housings 114 may be configured to enclose at least a portion of one or more control units 116. In some embodiments, one or more nitric oxide permeable housings 114 may be configured to enclose at least a portion of one or more nitric oxide sensors 120. In some embodiments, one or more nitric oxide permeable housings 114 may be configured to enclose one or more photolyzable nitric oxide donors 104. In some embodiments, one or more nitric oxide permeable housings 114 may be configured to enclose one or more light sources 106. In some embodiments, one or more nitric oxide permeable housings 114 may be configured to enclose one or more photolyzable nitric oxide donors 104 and one or more light sources 106. In some embodiments, one or more nitric oxide permeable housings 114 may be configured to enclose one or more photolyzable nitric oxide donors 104, one or more light sources 106, and one or more control units 116. In some embodiments, one or more nitric oxide permeable housings 114 may be configured to enclose one or more photolyzable nitric oxide donors 104, one or more Tight sources 106, one or more control units 116, and one or more electromagnetic receivers 108. In some embodiments, one or more nitric oxide permeable housings 114 may be configured to enclose one or more photolyzable nitric oxide donors 104, one or more light sources 106, one or more control units 116, one or more electromagnetic receivers 108, or substantially any combination thereof. In some embodiments, one or more nitric oxide permeable housings 114 may be configured to include one or more compartments. For example, in some embodiments, a nitric oxide permeable housing 114 may include a compartment that is configured to accept one or more light sources 106 and a second compartment that is configured to accept one or more photolyzable nitric oxide donors 104. In some embodiments, a nitric oxide permeable housing 114 may include a compartment that is configured to accept one or more light sources 106, a light permeable divider, and a second compartment that is configured to accept one or more photolyzable nitric oxide donors 104. In some embodiments, a light permeable divider may be made of a material that allows light to pass through the divider. Examples of such material include, but are not limited to, plastic, quartz, and the like.

Nitric oxide permeable housings 114 may be constructed of numerous types of materials and combinations of materials. Examples of such materials include, but are not limited to, ceramics polymeric materials, metals, plastics, and the like. In some embodiments, nitric oxide permeable housings 114 may include numerous combinations of materials.

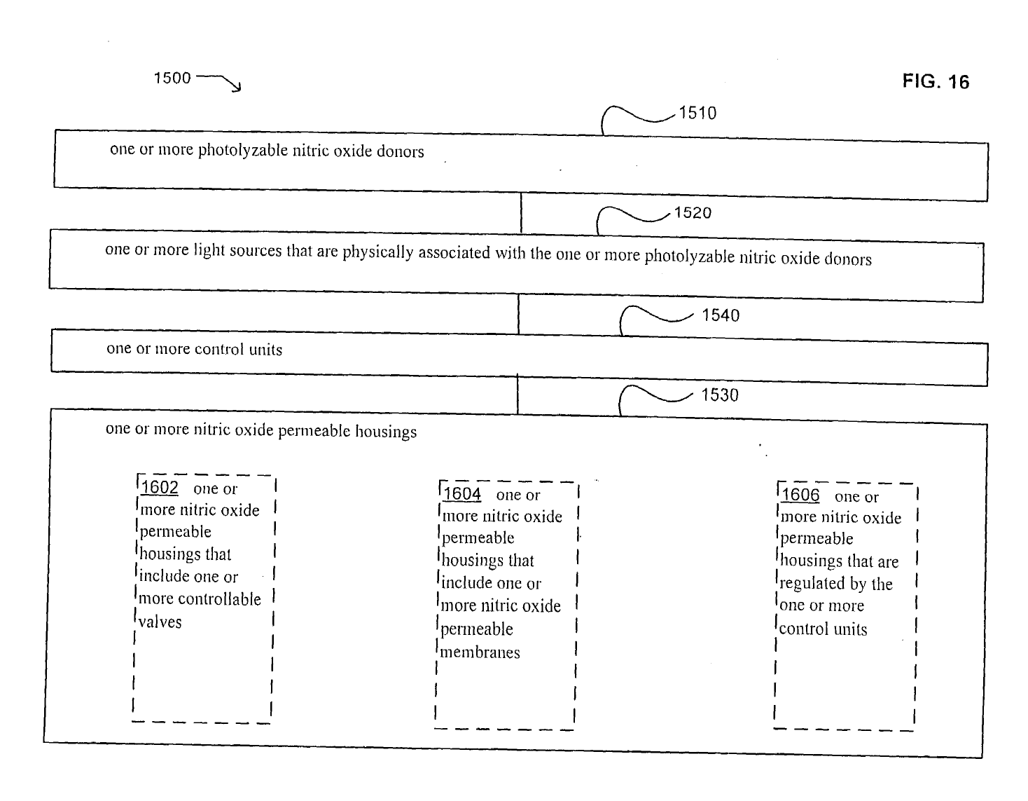

FIG. 16 illustrates alternative embodiments of embodiment 1500 of device 102 within system 100 of FIG. 15. FIG. 16 illustrates example embodiments of module 1530. Additional embodiments may include an embodiment 1602, an embodiment 1604, and/or an embodiment 1606.

At embodiment 1602, module 1530 may include one or more nitric oxide permeable housings that include one or more controllable valves. In some embodiments, one or more nitric oxide permeable housings 114 may include one or more controllable valves. In some embodiments, a controllable valve may provide for the passage of nitric oxide. In some embodiments, a controllable valve may provide for the passage of one or more photolyzable nitric oxide donors 104. In some embodiments, one or more controllable valves may include one or more electromagnets that provide for controlled opening and closing of an orifice associated with the valve. In some embodiments, one or more controllable valves may include one or more screw closures that provide for controlled opening and closing of the valve. For example, in some embodiments, a nitric oxide permeable housing 114 may include an electric motor that operates the screw mechanism to provide for opening and closure of an orifice associated with the nitric oxide permeable housing 114. Numerous controllable valves may be associated with one or more nitric oxide permeable housings 114. In some embodiments, a nitric oxide permeable housing 114 may include one or more valves that are controllable by one or more control units 116. Accordingly, in some embodiments, valves may be opened or closed in response to one or more nitric oxide sensors 120, one or more signals 118, one or more information packets, one or more management units 122, or substantially any combination thereof.

At embodiment 1604, module 1530 may include one or more nitric oxide permeable housings that include one or more nitric oxide permeable membranes. In some embodiments, one or more nitric oxide permeable housings 114 may include one or more nitric oxide permeable membranes. For example, in some embodiments, a nitric oxide permeable housing 114 may include a nitric oxide impermeable metal canister that is coupled to a nitric oxide permeable membrane (e.g., U.S. Patent Application No.: 20020026937). In some embodiments, a nitric oxide permeable housing 114 may include a selectively permeable membrane. For example, in some embodiments, a nitric oxide permeable housing 14 may include a selectively permeable, hydrophilic polyester co-polymer membrane system that includes a copolymer with 70% polyester and 30% polyether (e.g., Sympatex™ 10 μm membrane, see Hardwick et al., Clinical Science, 100:395-400 (2001)). In some embodiments, a nitric oxide permeable housing 114 may include a scintered glass portion that is permeable to nitric oxide. Accordingly, nitric oxide permeable housings 114 may include numerous types of porous ceramics that are permeable to nitric oxide. In some embodiments, a nitric oxide permeable housing 114 may include a nitric oxide permeable coating (e.g., U.S. Patent Application Nos.: 2005022083S and 20030093143).

At embodiment 1606, module 1530 may include one or more nitric oxide permeable housings that are regulated by the one or more control units. In some embodiments, one or more nitric oxide permeable housings 114 may include one or more nitric oxide permeable housings 114 that are regulated by the one or more control units 116. For example, in some embodiments, one or more nitric oxide permeable housings 114 may include one or more controllable valves that are regulated (e.g., opened and/or closed) in response to one or more control units 116.

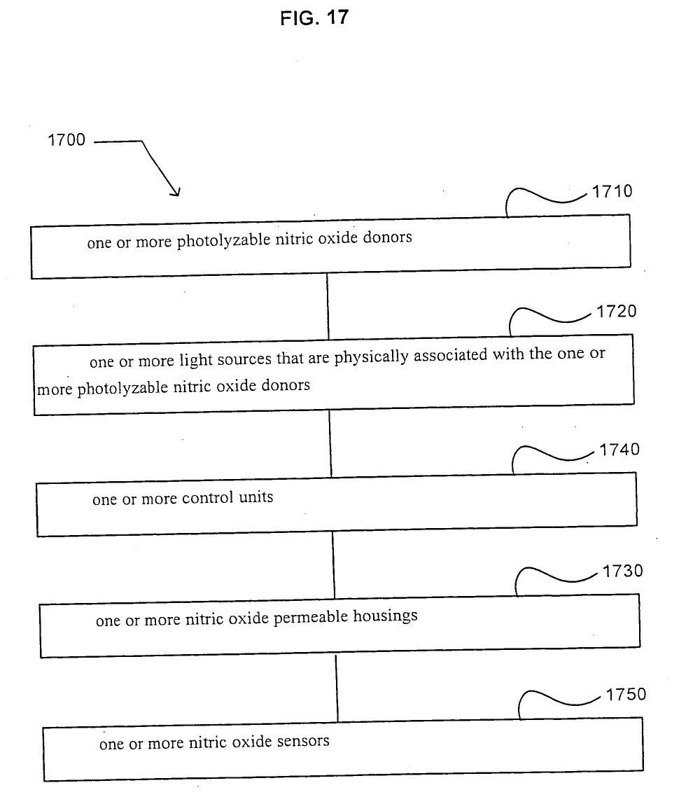

FIG. 17 illustrates alternative embodiment 1700 of device 102 within system I 00 of FIG. 1. In FIG. 17, discussion and explanation may be provided with respect to the above-described example of FIG. 1, and/or with respect to other examples and contexts. In some embodiments, modules 1510, 1520, 1530, and 1540 as described with respect to embodiment 1500 of device 102 of FIG. 15 may correspond to modules 1710, 1720, 1730, and 1740 as described with respect to embodiment 1700 of device 102 within system 100. However, it should be understood that the modules may execute operations in a number of other environments and contexts, and/or modified versions of FIG. 1 Also, although the various modules are presented in the sequence(s) illustrated, it should be understood that the Various modules may be configured in numerous orientations.

The embodiment 1700 includes module 1710 that includes one or more photolyzable nitric oxide donors. In some embodiments, a device 102 includes one or more photolyzable nitric oxide donors 104 that release nitric oxide upon photolysis. Examples of such photolyzable nitric oxide donors 104 include, but are not limited to, diazemiumdiolates (e.g., U.S. Pat. Nos. 7,105,502; 7,122,529; 6,673,338; herein incorporated by reference), trans-[RuCl([[15]aneN4)NO]+2 (Ferezin et al., Nitric Oxide, 13:170-175 (2005), Bonaventura et al., Nitric Oxide, 10:83-91 (2004)), nitrosyl, ligands (e.g., U.S. Pat. No. 5,665,077; herein incorporated by reference, Chmura et al., Nitric Oxide, 15:370-379 (2005), Flitney et al., Br. J. Pharmacol., 107:842-848 (1992), Flitney et al., Br. J. Pharmacol., 117:1549-1557 (1996), Matthew,s et al., Br. J. Pharmacol., 113:87-94 (1994)), 6-Nitrobenzo[a]pyrene (e.g., Fukuhara et al., J. Am. Chem. Soc., 123:8662-8666 (2001)), S-nitroso-glutathione (e.g., Rotta et al., Braz. J. Med. Res., 36:587-594 (2003), Flitney and Megson, J. Physiol., 550:819-828 (2003)), S-nitrosothiols (e.g., Andrews et al., British Journal of Pharmacology, 138:932-940 (2003), Singh et al., FEBS Lett., 360:47-51 (1995)), 2-Methyl-2-nitrosopropane (e.g., Pou et al., Mol. Pharm. 46:709-715 (1994), Wang et al., Chem. Rev., 102:1091-1134 (2002)), imidazolyl derivatives (e.g., U.S. Pat. No. 5,374,710; herein incorporated by reference).

The embodiment 1700 includes module 1720 that includes one or more light sources that are physically associated with the one or more photolyzable nitric oxide donors. In some embodiments, device 102 includes one or more light sources 106 that are physically associated with one or more photolyzable nitric oxide donors 104. In some embodiments, the one or more light sources 106 may be directly coupled to one or more photolyzable nitric oxide donors 104. For example, in some embodiments, the one or more photolyzable nitric oxide donors 104 may be chemically coupled to a surface of the light source 106 (e.g., chemically coupled to a polymer coating on the light source). In some embodiments, one or more photolyzable nitric oxide donors 104 may be indirectly coupled to one or more light sources 106. For example, in some embodiments, one or more photolyzable nitric oxide donors 104 may be coupled to a material that is used to coat the one or more light sources 106.

The embodiment 1700 includes module 1740 that includes one or more control units. In some embodiments, device 102 includes one or more control units 116. A device 102 may include numerous types of control units 116. In some embodiments, one or more control units 116 may be operably coupled with one or more light sources 106, one or more nitric oxide sensors 120, one or more electromagnetic receivers 108, one or more electromagnetic transmitters 112, or substantially an), combination thereof. In some embodiments, one or more control units 116 may be operably coupled to other components through use of one or more wireless connections, one or more hardwired connections, or substantially any combination thereof. Control units 116 may be configured in numerous ways. For example, in some embodiments, a control unit 116 may be configured as an on/off switch. Accordingly, in some embodiments, a control unit 116 may be configured to turn a light source 106 on and/or off. In some embodiments, a control unit 116 may be configured to control the emission of light from one or more light sources 106. For example, in some embodiments, one or more control units 116 may regulate the intensity of light emitted form one or more light sources 106, the duration of light emitted from one or more light sources 106, the frequency of light emitted from one or more light sources 106, wavelengths of light emitted from one or more light sources 106, or substantially any combination thereof. In some embodiments, one or more control units 116 may be configured to receive one or more signals 118 from one or more nitric oxide sensors 120. Accordingly, in some embodiments, one or more control units 116 may be configured to control one or more light sources 106 in response to one or more signals 118 received from one or more nitric oxide sensors 120. For example, in some embodiments, one or more nitric oxide sensors 120 may sense a low concentration of nitric oxide in one or more tissues and send one or more signals 118 to one or more control units 116. The one or more control units 116 may then turn one or more light sources 106 on to facilitate release of nitric oxide from one or more photolyzable nitric oxide donors 104. Accordingly, in some embodiments, one or more nitric oxide sensors 120 may sense a high concentration of nitric oxide in one or more tissues and send one or more signals 118 to one or more control units 116. The one or more control units 116 may then turn one or more light sources 106 off to end release of nitric oxide from one or more photolyzable nitric oxide donors 104. In some embodiments, one or, more control units 116 may be programmed to control one or more light sources 106. For example, in some embodiments, one or more control units 116 may be programmed to turn one or more light sources 106 on for a predetermined amount of time and then turn off. Accordingly, in some embodiments, one or more control units 116 may be preprogrammed. In some embodiments, one or more control units 116 may be dynamically programmed. For example, in some embodiments, one or more management units 122 may receive one or more signals 118 from one or more nitric oxide sensors 120 and program one or more control units 116 in response to the one or more signals 118 received from the one or more nitric oxide sensors 120. In some embodiments, one or more control units 116 may include one or more receivers that are able to receive one or more signals 118, one or more information packets, or substantially any combination thereof. Control units 116 may be configured in numerous ways. For example, in some embodiments, one or more control units 116 may be operably coupled to one or more light sources 106 that include numerous light emitting diodes that emit light of different wavelengths. Accordingly, in some embodiments, one or more control units 116 nay control the wavelengths of light emitted by the one or more light sources 106 by controlling the operation of light emitting diodes that emit light of the selected wavelength. Accordingly, control units 116 may be configured in numerous ways and utilize numerous types of mechanisms.

The embodiment 1700 includes module 1730 that includes one or more nitric oxide permeable housings. In some embodiments, device 102 may include one or more nitric oxide permeable housings 114. In some embodiments, nitric oxide permeable housings 114 may be configured for implantation within an individual 126. In some embodiments, nitric oxide permeable housings 114 may be configured to facilitate application of nitric oxide to a surface. In some embodiments, one or more nitric oxide permeable housings 114 may be configured to facilitate application of nitric oxide to one or more surfaces of an individual 126. For example, in some embodiments, one or more nitric oxide permeable housings 114 may be configured as a canister having a nitric oxide permeable end that mall be positioned on a skin surface of an individual 126 to delivery nitric oxide to the skill surface. In some embodiments, one or more nitric oxide permeable housings 114 mall be configured for insertion into the urethra of a male. In some embodiments, one or more nitric oxide permeable housings 114 may be configured for insertion into the vagina of a female.

In some embodiments, one or more nitric oxide permeable housings 114 may be configured to enclose at least a portion of one or more photolyzable nitric oxide donors 104. In some embodiments, one or more nitric oxide permeable housings 114 may be configured to enclose at least a portion of one or more light sources 106. In some embodiments, one or more nitric oxide permeable housings 114 may be configured to enclose at least a portion of one or more control units 116. In some embodiments, one or more nitric oxide permeable housings 114 may be configured to enclose at least a portion of one or more nitric oxide sensors 120. In some embodiments, one or more nitric oxide permeable housings 114 may be configured to enclose one or more photolyzable nitric oxide donors 104. In some embodiments, one or more nitric oxide permeable housings 114 may be configured to enclose one or more light sources 106. In some embodiments, one or more nitric oxide permeable housings 114 may be configured to enclose one or more photolyzable nitric oxide donors 104 and one or more light sources 106. In some embodiments, one or more nitric oxide permeable housings 114 may be configured to enclose one or more photolyzable nitric oxide donors 104, one or more light sources 106, and one or more control units 116. In some embodiments, one or more nitric oxide permeable housings 114 may be configured to enclose one or more photolyzable nitric oxide donors 104, one or more light sources 106, one or more control units 116, and one or more electromagnetic receivers 108. In some embodiments, one or more nitric oxide permeable housings 114 may be configured to enclose one or more photolyzable nitric oxide donors 104, one or more light sources 106, one or more control units 116, one or more electromagnetic receivers 108, or substantially an), combination thereof. In some embodiments, one or more nitric oxide permeable housings 114 may be configured to include one or more compartments. For example, in some embodiments, a nitric oxide permeable housing 114 may include a compartment that is configured to accept one or more light sources 106 and a second compartment that is configured to accept one or more photolyzable nitric oxide donors 104. In some embodiments, a nitric oxide permeable housing 114 may include a compartment that is configured to accept one or more light sources 106, a light permeable divider, and a second compartment that is configured to accept one or more photolyzable nitric oxide donors 104. In some embodiments, a light permeable divider may be made of a material that allows light to pass through the divider. Examples of such material include, but are not limited to, plastic quartz, and the like.

Nitric oxide permeable housings 114 may be constructed of numerous types of materials and combinations of materials. Examples of such materials include, but are not limited to, ceramics, polymeric materials, metals, plastics, and the like. In some embodiments, nitric oxide permeable housings 114 may include numerous combinations of materials.

The embodiment 1700 includes module 1750 that includes one or more nitric oxide sensors. In some embodiments, device 10 includes one or more nitric oxide sensors 120. In some embodiments, one or more nitric oxide sensors 120 may be used to determine the presence of nitric oxide in one or more tissues. In some embodiments, a nitric oxide sensor 120 may be configured for use on the outside surface of an individual 126. For example, in some embodiments, one or more nitric oxide sensors 120 may be configured to detect the concentration of nitric oxide on the surface of skin, a wound, a surface of a table, and the like. In some embodiments, one or more nitric oxide sensors 120 may be configured to be included within one or more housings. In some embodiments, one or more nitric oxide sensors 120 may be configured to be included within one or more nitric oxide permeable housings 114. In some embodiments, a nitric oxide sensor 120 may be configured to utilize fluorescence to detect nitric oxide. For example, in some embodiments, a nitric oxide sensor may detect nitric oxide through use of one or more fluorescent probes, such as 4,5-diaminofluorescein diacetate (EMD Chemicals Inc., San Diego, Calif.). In some embodiments, a nitric oxide sensor may detect nitric oxide through use of one or more electrodes. For example, in some embodiments, a nitric oxide sensor may utilize an electrode that includes a single walled carbon nanotube and an ionic liquid to detect nitric oxide (e.g., Li et al., Electroanalysis, 18:713-718 (2006)). Numerous nitric oxide sensors 120 are commercially available and have been described (e.g., World Precision Instruments. Inc., Sarasota, Fla., USA; U.S. Pat. Nos. 6,100,096; 6,980,604: 5,980,705). In some embodiments, a nitric oxide sensor 120 may include one or more transmitters. In some embodiments, a nitric oxide sensor 120 may include one or more receivers. In some embodiments, a nitric oxide sensor 120 may be configured to transmit one or more signals 118. In some embodiments, a nitric oxide sensor 120 may be configured to receive one or more signals 118.

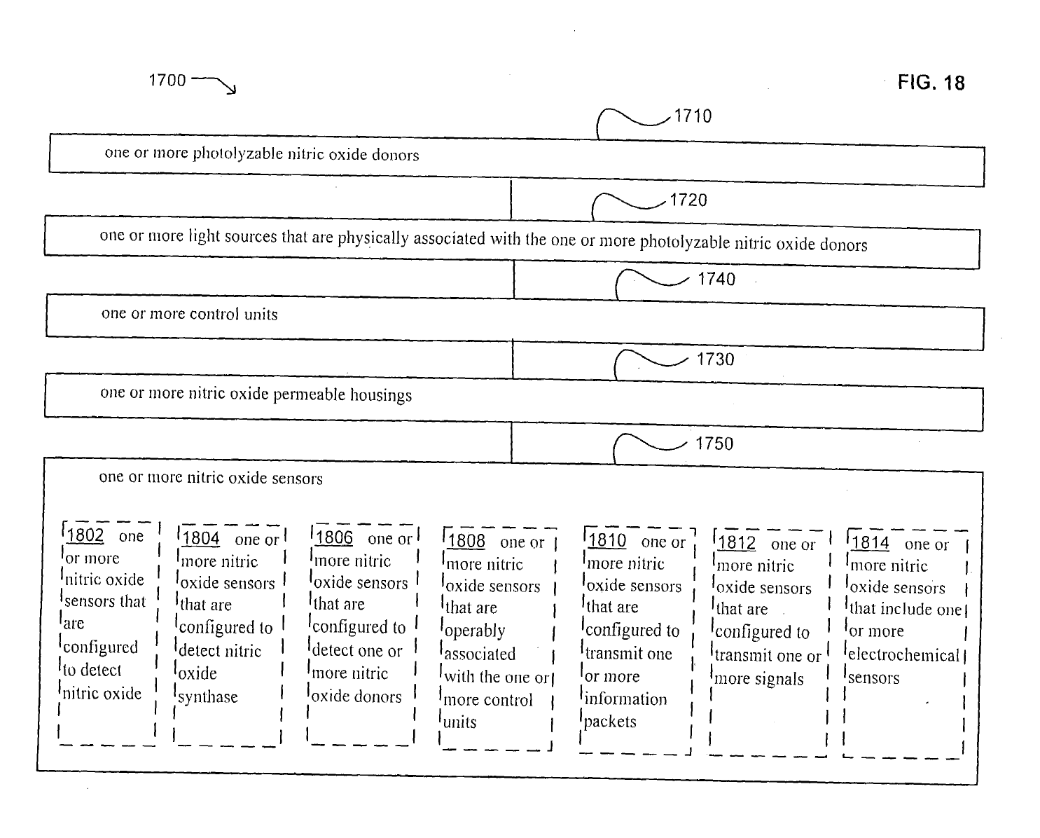

FIG. 18 illustrates alternative embodiments of embodiment 1700 of device 102 of FIG. 17. FIG. 18 illustrates example embodiments of module 1750. Additional embodiments mail include an embodiment 1802, an embodiment 1804, an embodiment 1806, an embodiment 1808, an embodiment 1810, an embodiment 1812, and/or an embodiment 1814.

At embodiment 1802, module 1750 may include one or more nitric oxide sensors that are configured to detect nitric oxide. In some embodiments, one or more nitric oxide sensors 120 may include one or more nitric oxide sensors 120 that are configured to detect nitric oxide. Nitric oxide sensors 120 may be configured in numerous ways. In some embodiments, a nitric oxide sensor 120 may be configured to utilize fluorescence to detect nitric oxide. For example, in some embodiments, a nitric oxide sensor may detect nitric oxide through use of one or more fluorescent probes, such as 4,5-diaminofluorescein diacetate (EMD Chemicals Inc., San Diego, Calif.). In some embodiments, a nitric oxide sensor may detect nitric oxide through use of one or more electrodes. For example, in some embodiments, a nitric oxide sensor may utilize an electrode that includes a single walled carbon nanotube and an ionic liquid to detect nitric oxide (e.g., Li et al., Electroanalysis, 18:713-718 (2006)). Numerous nitric oxide sensors 120 are commercially available and have been described (e.g., World Precision Instruments, Inc., Sarasota, Fla., USA; U.S. Pat. Nos. 6,100,096; 6,280,604; 5,980,705).

At embodiment 1804, module 1750 may include one or more nitric oxide sensors that are configured to detect nitric oxide synthase. In some embodiments, one or more nitric oxide sensors 120 may include one or more nitric oxide sensors 120 that are configured to detect one or more nitric oxide synthases. In some embodiments, one or more nitric oxide sensors 120 may be configured to detect nitric oxide synthase activity. Nitric oxide synthase detection kits are commercially available (e.g., Cell Technology, Inc., Mountain View, Calif.). In some embodiments, one or more nitric oxide sensors 120 may be configured to detect nitric oxide synthase messenger ribonucleic acid (mRNA). Methods that may be used to detect such mRNA have been reported (e.g., Sonoki et al., Leukemia, 13:713-718 (1999)). In some embodiments, one or more nitric oxide sensors 120 may be configured to detect nitric oxide synthase through immunological methods. Methods that may be used to detect nitric oxide synthase directly been reported (e.g., Burrell et al., J. Histochem. Cytochem., 44:339-346 (1996) and Hattenbach et al., Ophthalmologica, 216:209-214 (2002)). In some embodiments, microelectromechanical systems may be used to detect nitric oxide synthase. In some embodiments, antibodies and/or aptamers that bind to nitric oxide synthase mall be used within one or more microelectromechanical systems to detect nitric oxide synthase. Methods to construct microelectromechanical detectors have been described (e.g., Gau et al., Biosensors & Bioelectronics, 16:745-755 (2001)). Accordingly, nitric oxide sensors may be configured in numerous ways to detect one or more nitric oxide synthases.

At embodiment 1806, module 1750 may include one or more nitric oxide sensors that are configured to detect one or more nitric oxide donors. In some embodiments, one or more nitric oxide sensors 120 may include one or more nitric oxide sensors 120 that are configured to detect one or more nitric oxide donors. In some embodiments, one or more nitric oxide sensors 120 may include one or more surface plasmon resonance chemical electrodes that are configured to detect one or more nitric oxide donors. For example, in some embodiments, one or more nitric oxide sensors 120 may include one or more surface plasmon resonance chemical electrodes that include antibodies and/or aptamers that bind to one or more nitric oxide donors. Accordingly, such electrodes may be used to detect the one or more nitric oxide donors through use of surface plasmon resonance. Methods to construct surface plasmon resonance chemical electrodes are known and haste been described (e.g., U.S. Pat. No. 5,858,799; Lin et al., Applied Optics, 46:800-806 (2007)). In some embodiments, antibodies and/or aptamers that bind to one or more nitric oxide donors may be used within one or more microelectromechanical systems to detect one or more nitric oxide donors. Methods to construct microelectromechanical detectors have been described (e.g., Gau et al., Biosensors & Bioelectronics, 16:745-755 (2001)).

At embodiment 1808, module 1750 may include one or more nitric oxide sensors that are operably associated with the one or more control units. In some embodiments, a device 102 may include one or more nitric oxide sensors 120 that are operably associated with one or more control units 116. In some embodiments, one or more nitric oxide sensors 120 mall be operably associated with one or more control units 116 through a hardwired connection. In some embodiments, one or more nitric oxide sensors 120 may be operably associated with one or more control units 116 through a wireless connection. In some embodiments, one or more nitric oxide sensors 120 may be configured to send one or more signals 118 to one or more control units 116. In some embodiments, one or more nitric oxide sensors 120 may be configured to receive one or more signals 118 from one or more control units 116.

At embodiment 1810, module 1750 may include one or more nitric oxide sensors that are configured to transmit one or more information packets. In some embodiments, one or more nitric oxide sensors 120 may be configured to transmit one or more information packets. In some embodiments, one or more nitric oxide sensors 120 may be configured to transmit one or more information packets to one or more control units 116. Information packets may include numerous types of information. Examples of such information include, but are not limited to, nitric oxide concentration, temperature, time, and the like.

At embodiment 1812, module 1750 may include one or more nitric oxide sensors that are configured to transmit one or more signals. In some embodiments, one or more nitric oxide sensors 120 may be configured to transmit one or more signals 118. In some embodiments, one or more nitric oxide sensors 120 may be configured to transmit one or more signals 118. Numerous types of signals 118 may be transmitted. Examples of such signals 118 include, but are not limited to, optical signals 118, radio signals 118, wireless signals 118, hardwired signals 118, infrared signals 118, ultrasonic signals 118, and the like.

At embodiment 1814, module 1750 may include one or more nitric oxide sensors that include one or more electrochemical sensors. In some embodiments, a device 102 may include one or more nitric oxide sensors 120 that include one or more electrochemical sensors. Nitric oxide sensors 120 may include numerous types of electrochemical sensors. For example, in some embodiments; an electrochemical sensor may be configured as a nitric oxide specific electrode. In some embodiments, a nitric oxide specific electrode may include ruthenium and/or at least one oxide of ruthenium. Methods to construct such electrodes are known and have been described (e.g., U.S. Pat. Nos. 6,280,604; 5,980,705). In some embodiments, a nitric oxide sensor 120 may include an amperometric sensor that includes a sensing electrode that is configured to oxidize nitric oxide complexes to generate an electrical current that indicates the concentration of nitric oxide. Methods to construct such electrodes are known and have been described (e.g., U.S. Patent Application No.: 20070181444). Numerous types of electrochemical sensors may be associated with one or more nitric oxide sensors 120 (e.g., Li et al., Electroanalysis, 18:713-718 (2006)). Electrodes that may be used to detect nitric oxide are commercially available (World Precision Instruments, Sarasota, Fla.). In some embodiments, such electrodes may be used to detect nitric oxide at concentrations of about 0.5 nanomolar and above, and may be about 100 micrometers in diameter (World Precision Instruments, Sarasota, Fla.).

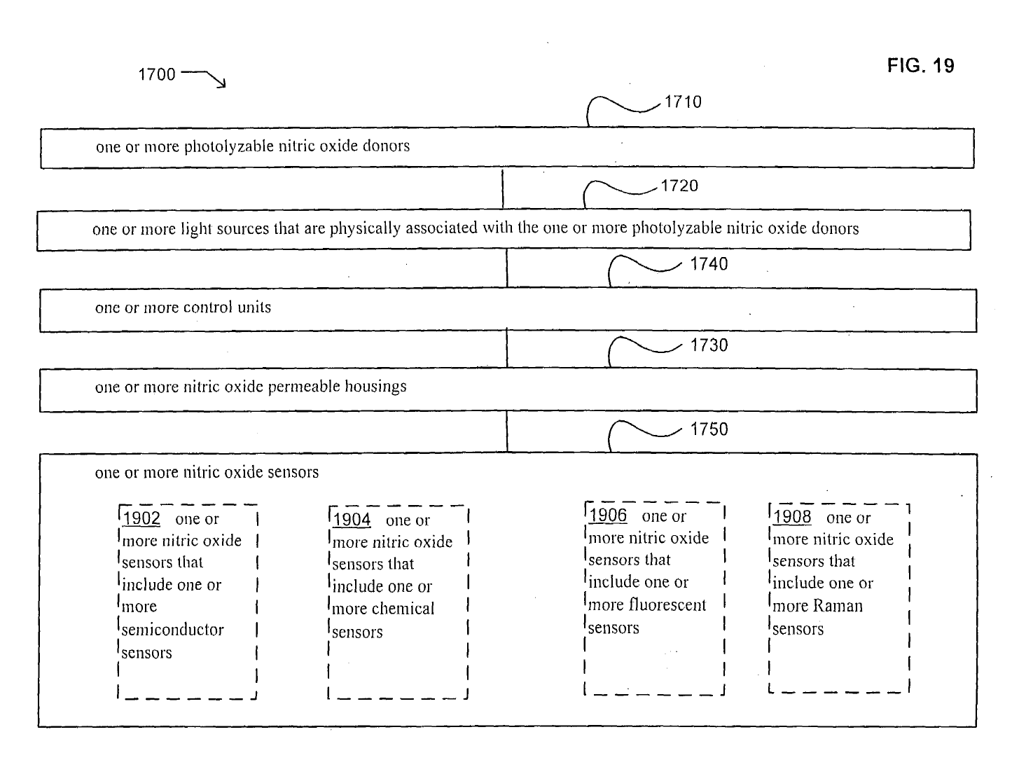

FIG. 19 illustrates alternative embodiments of embodiment 1700 of device 102 of FIG. 17. FIG. 19 illustrates example embodiments of module 1750. Additional embodiments may include an embodiment 1902, an embodiment 1904, an embodiment 1906, and/or an embodiment 1908.

At embodiment 1902, module 1750 may include one or more nitric oxide sensors that include one or more semiconductor sensors. In some embodiments, one or more nitric oxide sensors 120 may include one or more nitric oxide sensors 120 that include one or more semiconductor sensors. In some embodiments, the sensor may be a molecular controlled semiconductor resistor of a multilayered GaAs structure to which a layer of multifunctional NO-binding molecules are adsorbed. Such nitric oxide binding molecules may include, but are not limited to vicinal diamines, metalloporphyrins, metallophthalocyanines, and iron-dithiocarbamate complexes that contain at least one functional group selected from carboxyl, thiol, acyclic sulfide, cyclic disulfide, hydroxamic acid, trichlorosilane or phosphate (e.g., U.S. Published Patent Application No.: 20040072360). In some embodiments, a semiconductive nitric oxide sensor 120 may employ a polycrystalline-oxide semiconductor material that is coated with porous metal electrodes to form a semiconductor sandwich. In some embodiments, the semiconductor material may be formed of SnO₂ or ZnO. The porous electrodes may be formed with platinum and used to measure the conductivity of the semiconductor material. The conductivity of the semiconductor material changes when nitric oxide is absorbed on the surface of the semiconductor material (e.g., U.S. Pat. No. 5,580,433). Numerous other semiconductor sensors may be used to detect nitric oxide.

At embodiment 1904, module 1750 may include one or more nitric oxide sensors that include one or more chemical sensors. In some embodiments, a device 102 may include one or more nitric oxide sensors 120 that include one or more chemical sensors. For example, in some embodiments, one or more nitric oxide sensors 120 may include one or more chemical sensors that include a reagent solution that undergoes a chemiluminescent reaction with nitric oxide. Accordingly, one or more photodetectors may be used to detect nitric oxide. Methods to construct such detectors are known and have been described (e.g., U.S. Pat. No. 6,100,096). In some embodiments, ozone may be reacted with nitric oxide to produce light in proportion to the amount of nitric oxide present. The light produced may be measured with a photodetector. In some embodiments, sensors may include one or more charge-coupled devices to detect photonic emission.

At embodiment 1906, module 1750 may include one or more nitric oxide sensors that include one or more fluorescent sensors. In some embodiments, a device 102 may include one or more nitric oxide sensors 120 that include one or more fluorescent sensors. In some embodiments, a fluorescent sensor may include one or more fluorescent probes that may be used to detect nitric oxide. For example, in some embodiments, 4,5-diaminofluorescein may be used to determine nitric oxide concentration (e.g., Rathel et al., Biol. Proced. Online, 5:136-142 (2003)). Probes that may be used to detect nitric oxide are commercially available (EMD Chemicals Inc., San Diego, Calif.).

At embodiment 1908, module 1750 may include one or more nitric oxide sensors that include one or more Raman sensors. In some embodiments, one or more nitric oxide sensors 120 may include one or more nitric oxide sensors 120 that include one or more Raman sensors. Methods to use Raman spectroscopy to detect nitric oxide are known and have been described (e g., U.S. Patent Application No.: 20060074282). In addition, Raman spectrometers are commercially available (e.g., Raman Systems, Austin, Tex. and B&W Tek. Inc., Newark, Del.).

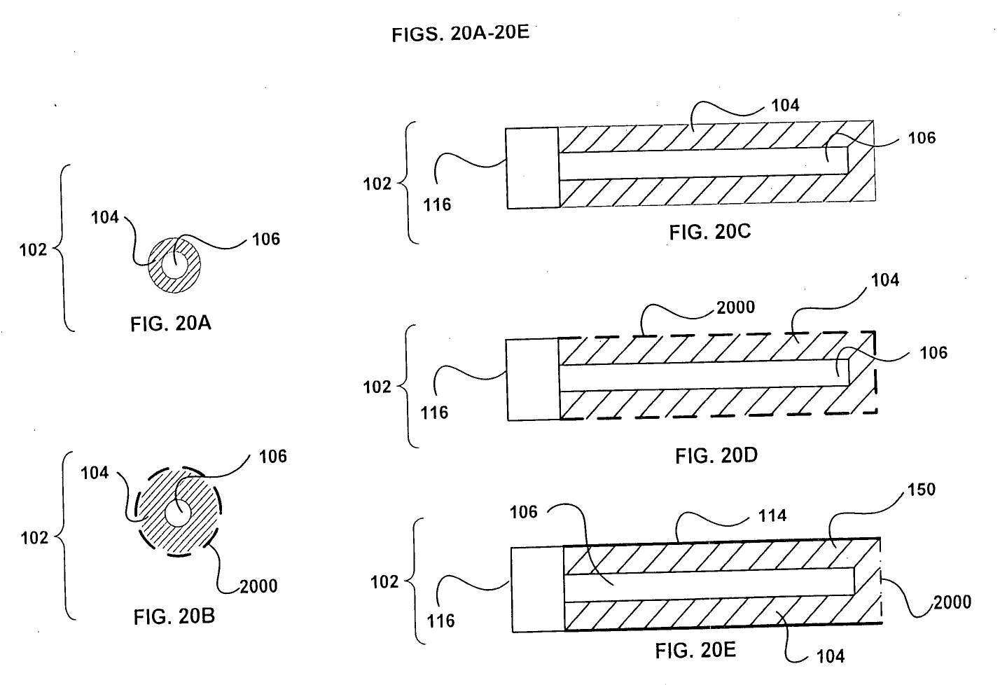

FIG. 20A illustrates an embodiment of device 102. Photolyzable nitric oxide donor 104 is shown in association with light source 106.

FIG. 20B illustrates an embodiment of device 102. Photolyzable nitric oxide donor 104 is shown in association with light source 106 and nitric oxide permeable membrane 2000.

FIG. 20C illustrates an embodiment of device 102. Photolyzable nitric oxide donor 104 is shown in association with light source 106, and control unit 116.

FIG. 20D illustrates an embodiment of device 102. Photolyzable nitric oxide donor 104 is shown in association with light source 106, control unit 116, and nitric oxide permeable membrane 2000.

FIG. 20E illustrates an embodiment of device 102. Photolyzable nitric oxide donor 104 is shown in association with light source 106, control unit 116, and nitric oxide permeable housing 114 that includes a nitric oxide permeable membrane 2000.

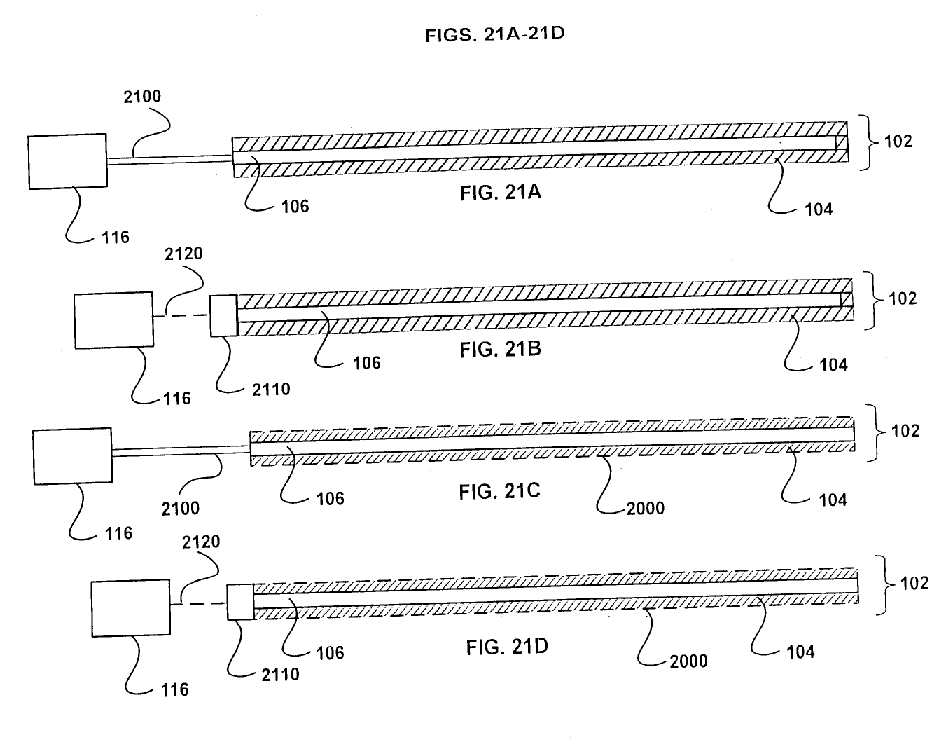

FIG. 21A illustrates an embodiment of device 102. Photolyzable nitric oxide donor 104 is shown in association with light source 106, and control unit 116. Control unit 116 is shown associated with light source 106 through a hardwired connection 2100.

FIG. 21B illustrates an embodiment of device 102. Photolyzable nitric oxide donor 104 is shown in association with light source 106, control unit 116, and receiver 2110. Control unit 116 is shown associated with receiver 2110 through a wireless connection 2120.

FIG. 21C illustrates an embodiment of device 102. Photolyzable nitric oxide donor 104 is shown in association with light source 106, control unit 116, and nitric oxide permeable membrane 2000. Control unit 116 is shown associated with light source 106 through a hardwired connection 2100.

FIG. 21D illustrates an embodiment of device 102. Photolyzable nitric oxide donor 104 is shown in association with light source 106, control unit 116, nitric oxide permeable membrane 2000, and receiver 2110. Control unit 116 is shown associated with receiver 2110 through a wireless connection 2120.

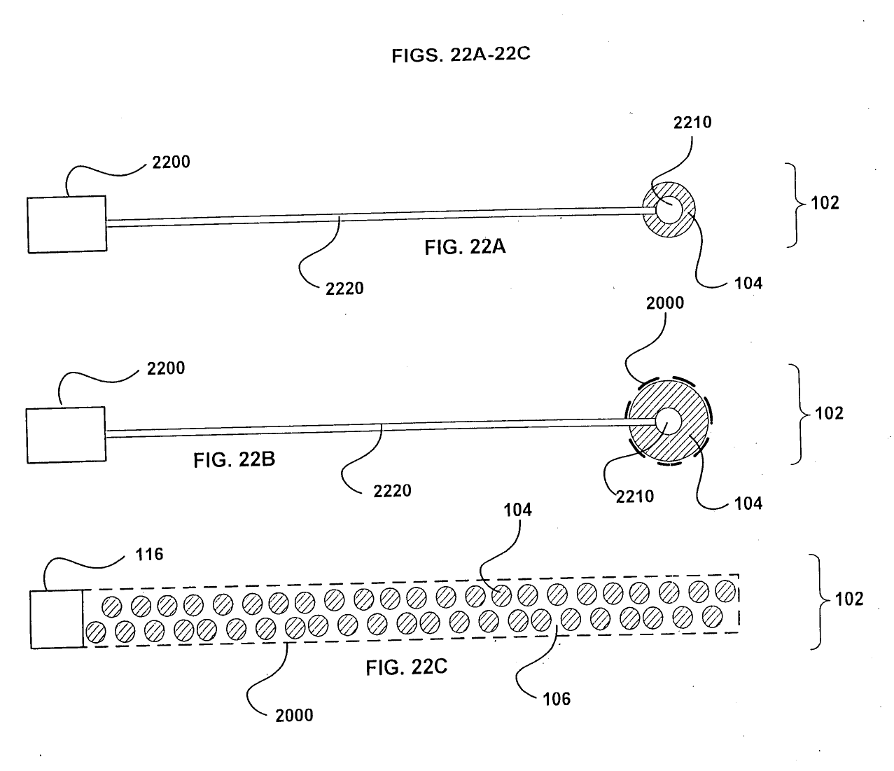

FIG. 22A illustrates an embodiment of device 102. Photolyzable nitric oxide donor 104 is shown in association with light emitter 2210. Battered 2200 is shown associated with light emitter 2210 through a hardwired connection 2220.

FIG. 22B illustrates an embodiment of device 102. Photolyzable nitric oxide donor 104 is shown in association with light emitter 2210 and nitric oxide permeable membrane 2000. Battery 2200 is shown associated with light emitter 2210 through a hardwired connection 2220.

FIG. 22C illustrates an embodiment of device 102. Photolyzable nitric oxide donor 104 is shown in association with light source 106, nitric oxide permeable membrane 2000, and control unit 116.

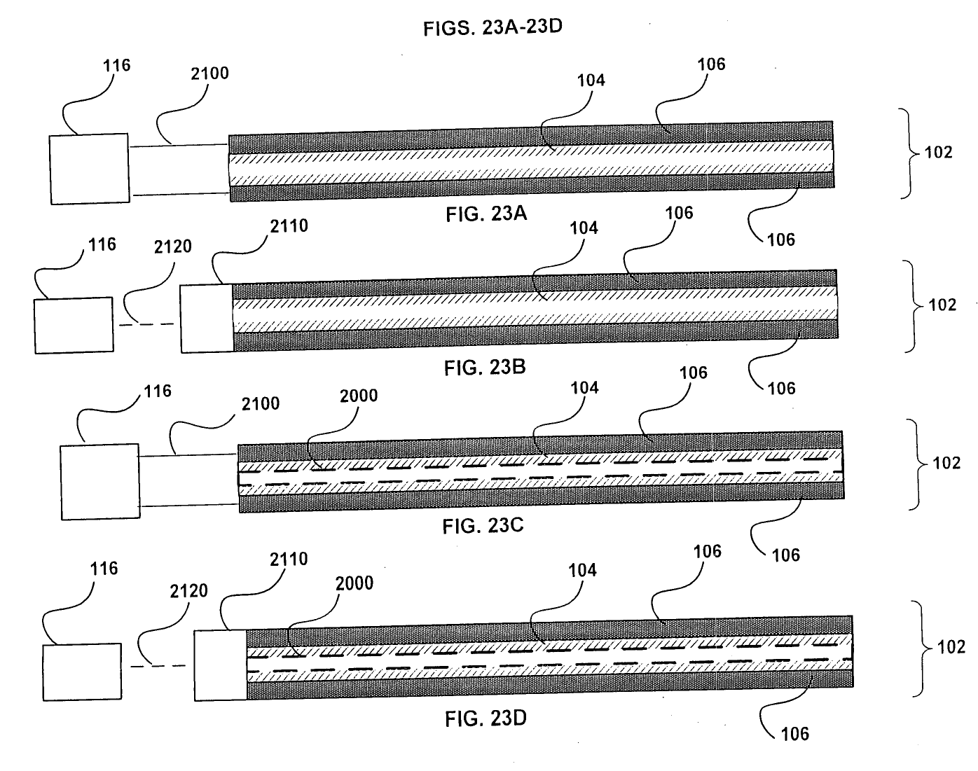

FIG. 23A illustrates an embodiment of device 102. Photolyzable nitric oxide donor 104 is shown in association width light source 106. Control unit 116 is shown associated with light source 106 through a hardwired connection 2100.

FIG. 23B illustrates an embodiment of device 102. Photolyzable nitric oxide donor 104 is shown in association with light source 106 and receiver 2110. Control unit 116 is shown associated with receiver 2110 through a wireless connection 2120.

FIG. 23C illustrates an embodiment of device 102. Photolyzable nitric oxide donor 104 is shown in association with light source 106 and nitric oxide permeable membrane 2000. Control unit 116 is shown associated with light source 106 through a hardwired connection 2100.

FIG. 23D illustrates an embodiment of device 102. Photolyzable nitric oxide donor 104 is shown in association with light source 106, receiver 2110 and nitric oxide permeable membrane 2000. Control unit 116 is shown associated with receiver 2110 through a wireless connection 2120.

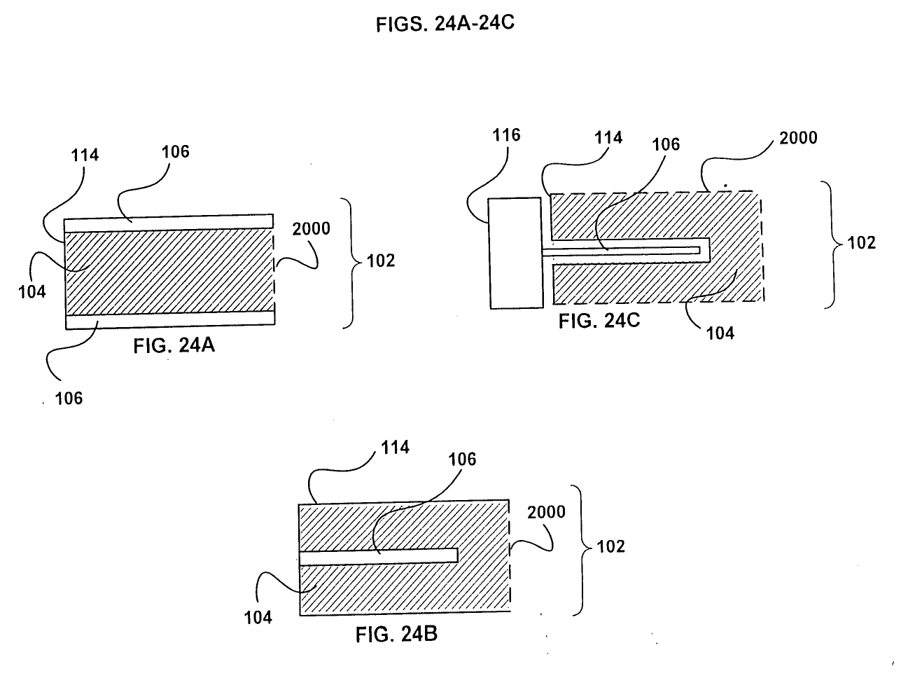

FIG. 24A illustrates an embodiment of device 102. Photolyzable nitric oxide donor 104 is shown in association with light source 106. The photolyzable nitric oxide donor 104 and light source 106 are enclosed within a nitric oxide permeable housing 114 that includes a nitric oxide permeable membrane 2000.

FIG. 24B illustrates an embodiment of device 102. Photolyzable nitric oxide donor 104 is shown in association with light source 106. The photolyzable nitric oxide donor 104 and light source 106 are enclosed within a nitric oxide permeable housing 114 that includes a nitric oxide permeable membrane 2000.

FIG. 24C illustrates an embodiment of device 102. Photolyzable nitric oxide donor 104 is shown enclosed within a nitric oxide permeable housing 114 that includes a nitric oxide permeable membrane 2000. Light source 106 is shown as being positioned within a cavity of the nitric oxide permeable housing 114 and associated with a control unit 116.

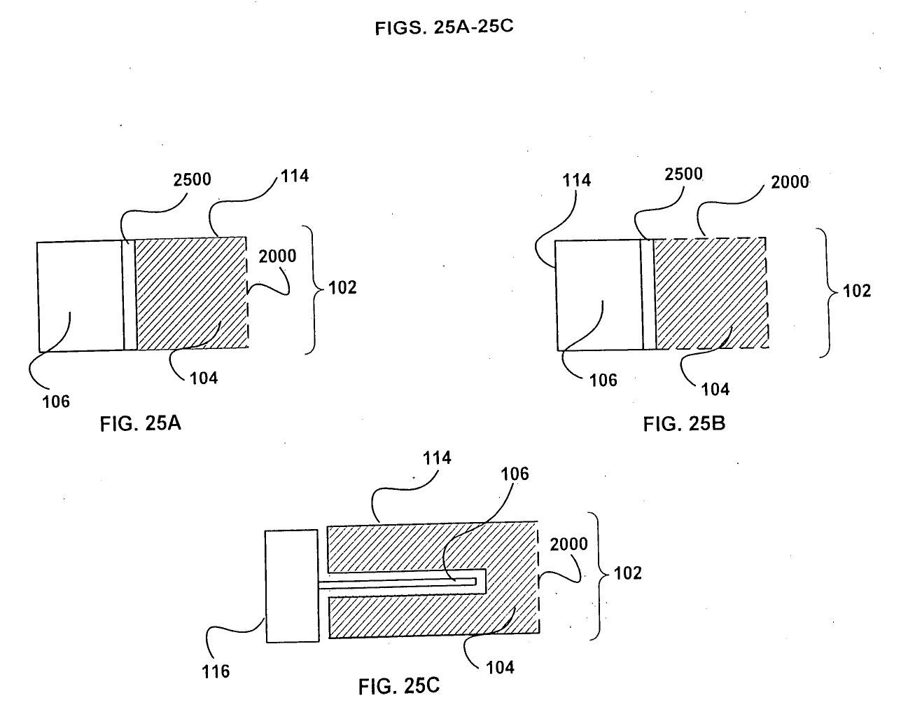

FIG. 25A illustrates an embodiment of device 102. Photolyzable nitric oxide donor 104 is shown enclosed within a nitric oxide permeable housing 114 that includes a nitric oxide permeable membrane 2000. Light source 106 is shown as being positioned within the nitric oxide permeable housing 114 in association with a light permeable barrier 2500.

FIG. 25B illustrates an embodiment of device 102. Photolyzable nitric oxide donor 104 is shown enclosed within a nitric oxide permeable housing 114 that includes a nitric oxide permeable membrane 2000. Light source 106 is shown as being positioned within the nitric oxide permeable housing 114 in association with a light permeable barrier 2500.

FIG. 25C illustrates an embodiment of device 102. Photolyzable nitric oxide donor 104 is shown enclosed within a nitric oxide permeable housing 114 that includes a nitric oxide permeable membrane 2000. Light source 106 is shown as being positioned within a cavity of the nitric oxide permeable housing 114 and associated with a control unit 116.

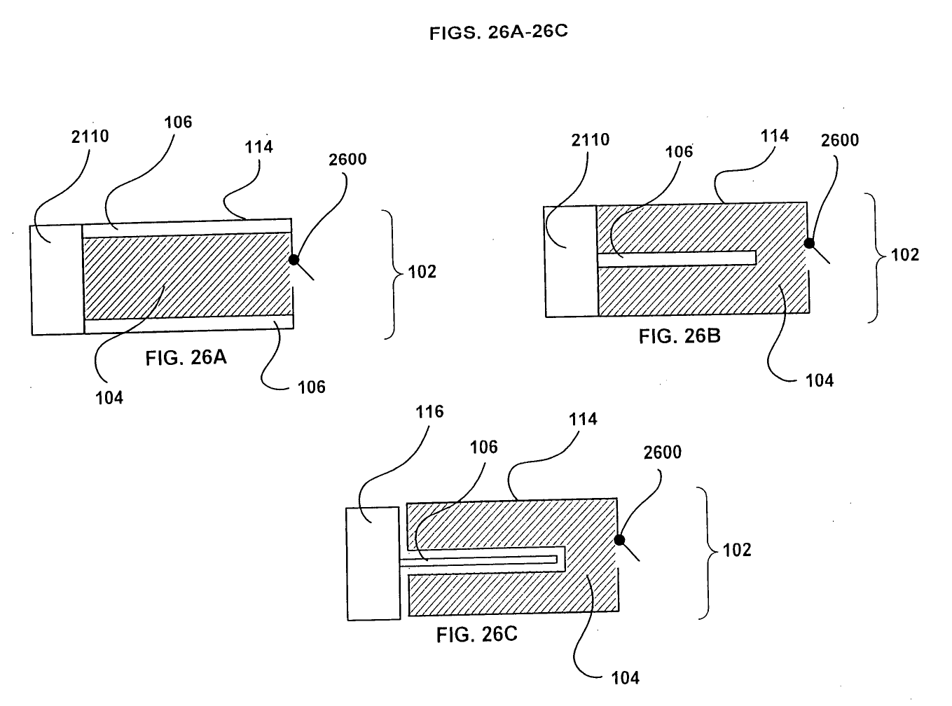

FIG. 26A illustrates an embodiment of device 102. Photolyzable nitric oxide donor 104 is shown enclosed within a nitric oxide permeable housing 114 that includes a controllable valve 2600. Light source 106 is shown as being positioned within the nitric oxide permeable housing 114. Receiver 2110 is associated with the nitric oxide permeable housing 114.

FIG. 26B illustrates an embodiment of device 102. Photolyzable nitric oxide donor 104 is shown enclosed within a nitric oxide permeable housing 114 that includes a controllable valve 2600. Light source 106 is shown as being positioned within the nitric oxide permeable housing 114. Receiver 2110 is associated with the nitric oxide permeable housing 114.

FIG. 26C illustrates an embodiment of device 102. Photolyzable nitric oxide donor 104 is shown enclosed within a nitric oxide permeable housing 114 that includes a controllable valve 2600. Light source 106 is shown as being positioned within a cavity of the nitric oxide permeable housing 114 and associated with a control unit 116.

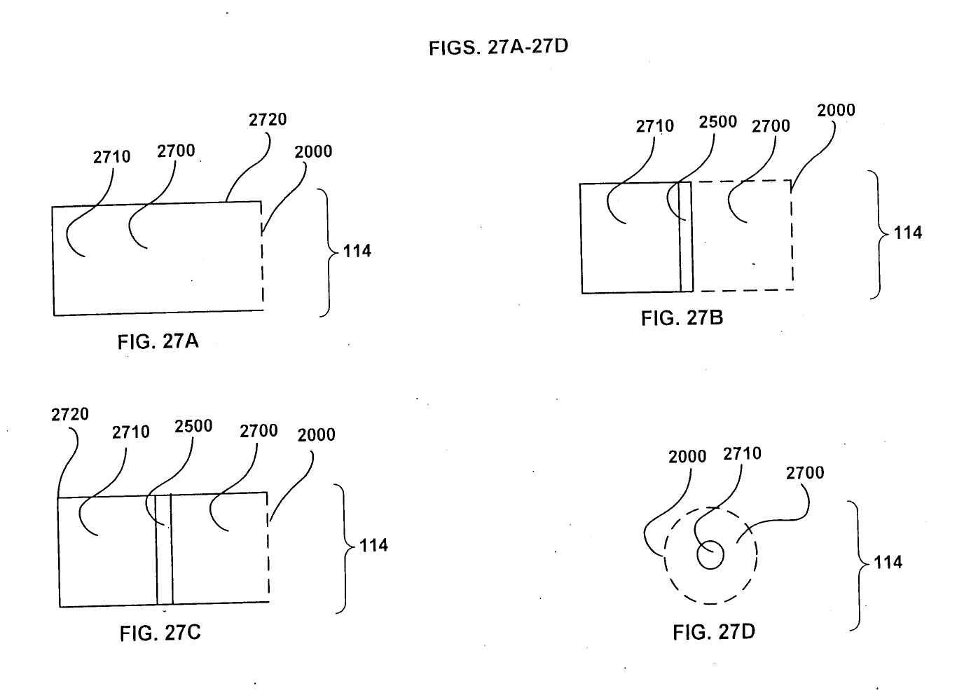

FIG. 27A illustrates an embodiment of nitric oxide permeable housing 114. Nitric oxide permeable housing 114 is illustrated as including a nitric oxide impermeable portion 2720 of the nitric oxide permeable housing 114 and a nitric oxide permeable membrane 2000. The nitric oxide permeable housing 114 includes a cavity 2710 configured to accept one or more light sources 106 and a cavity 2700 configured to accept one or more photolyzable nitric oxide donors 104.

FIG. 27B illustrates an embodiment of nitric oxide permeable housing 114. Nitric oxide permeable housing 114 is illustrated as including a nitric oxide permeable membrane 2000 that includes a cavity 2700 configured to accept one or more photolyzable nitric oxide donors 104. The nitric oxide permeable housing 114 includes a cavity 2710 configured to accept one or more light sources 106. Nitric oxide permeable housing 114 includes a light permeable barrier 2500 that separates the cavity 2700 configured to accept one or more photolyzable nitric oxide donors 104 from the cavity 2710 configured to accept one or more light sources 106.

FIG. 27C illustrates an embodiment of nitric oxide permeable housing 114. Nitric oxide permeable housing 114 is illustrated as including a nitric oxide impermeable portion 2720 of the nitric oxide permeable housing 114 and a nitric oxide permeable membrane 2000. Nitric oxide permeable housing 114 is illustrated as including a cavity 2700 configured to accept one or more photolyzable nitric oxide donors 104. The nitric oxide permeable housing 114 includes a cavity 2710 configured to accept one or more light sources 106. Nitric oxide permeable housing 114 includes a light permeable barrier 2500 that separates the cavity 2700 configured to accept one or more photolyzable nitric oxide donors 104 from the cavity 2710 configured to accept one or more light sources 106.

FIG. 27D illustrates an embodiment of nitric oxide permeable housing 114. Nitric oxide permeable housing 114 is illustrated as including a nitric oxide permeable membrane 2000. Nitric oxide permeable housing 114 includes a cavity 2700 configured to accept one or more photolyzable nitric oxide donors 104. The nitric oxide permeable housing 114 includes a cavity 2710 configured to accept one or more light sources 106.

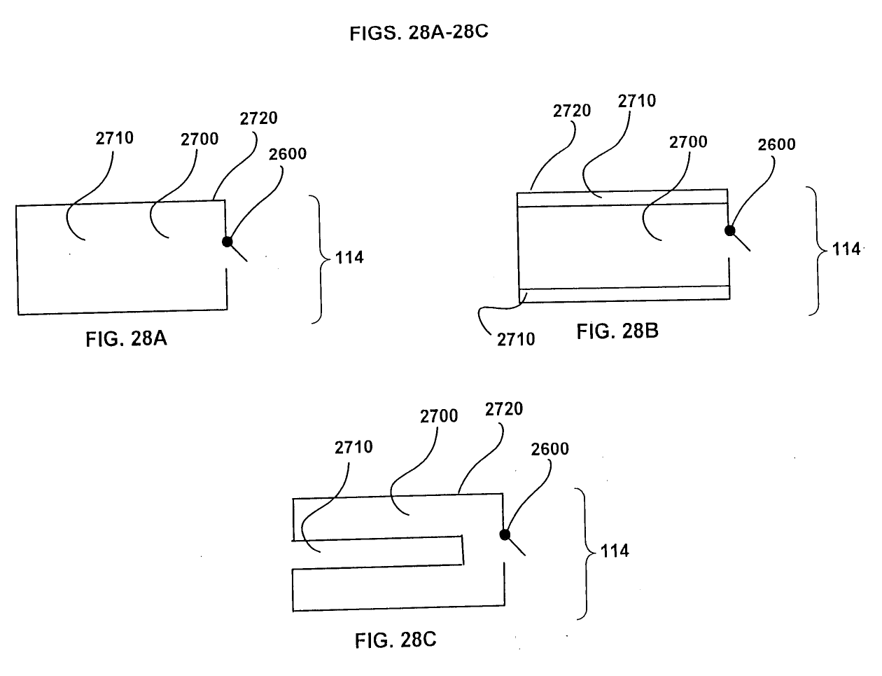

FIG. 28A illustrates an embodiment of nitric oxide permeable housing 114. Nitric oxide permeable housing 114 is illustrated as including a nitric oxide impermeable portion 2720 of the nitric oxide permeable housing 114 and a controllable valve 2600. The nitric oxide permeable housing 114 includes a cavity 2710 configured to accept one or more light sources 106 and a cavity 2700 configured to accept one or more photolyzable nitric oxide donors 104.

FIG. 28B illustrates an embodiment of nitric oxide permeable housing 114. Nitric oxide permeable housing 114 is illustrated as including a nitric oxide impermeable portion 2720 of the nitric oxide permeable housing 114 and a controllable valve 2600. The nitric oxide permeable housing 114 includes a cavity 2710 configured to accept one or more light sources 106 and a cavity 2700 configured to accept one or more photolyzable nitric oxide donors 104.

FIG. 28C illustrates an embodiment of nitric oxide permeable housing 114. Nitric oxide permeable housing 114 is illustrated as including a nitric oxide impermeable portion 2720 of the nitric oxide permeable housing 114 and a controllable valve 2600. The nitric oxide permeable housing 114 includes a cavity 2710 configured to accept one or more light sources 106 and a cavity 2700 configured to accept one or more photolyzable nitric oxide donors 104.

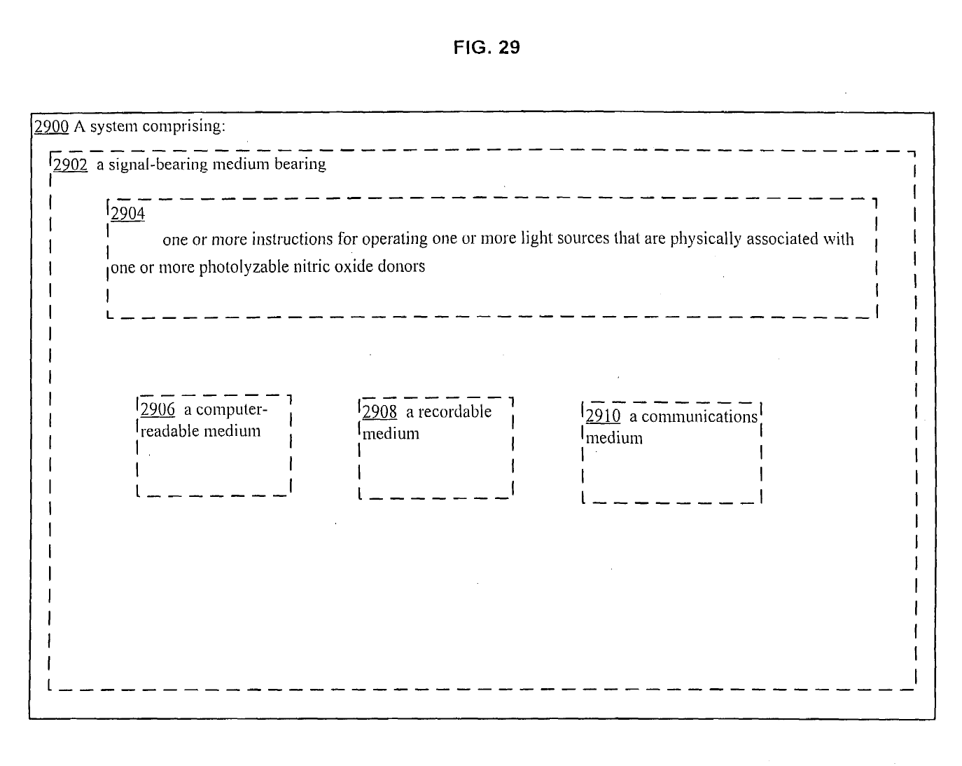

FIG. 29 illustrates a partial view of a system 2900 that includes a computer program 2904 for executing a computer process on a computing device. An embodiment of the system 2900 is provided using a signal-bearing medium 2902 bearing at least one of one or more instructions for operating one or more light sources that are physically associated with one or more photolyzable nitric oxide donors. The one or more instructions may be, for example, computer executable and/or logic-implemented instructions. In some embodiments, the signal-bearing medium 2902 may include a computer-readable medium 2906. In some embodiments, the signal bearing medium 2909 may include a recordable medium 2908. In some embodiments, the signal bearing medium 2902 may include a communications medium 2910.

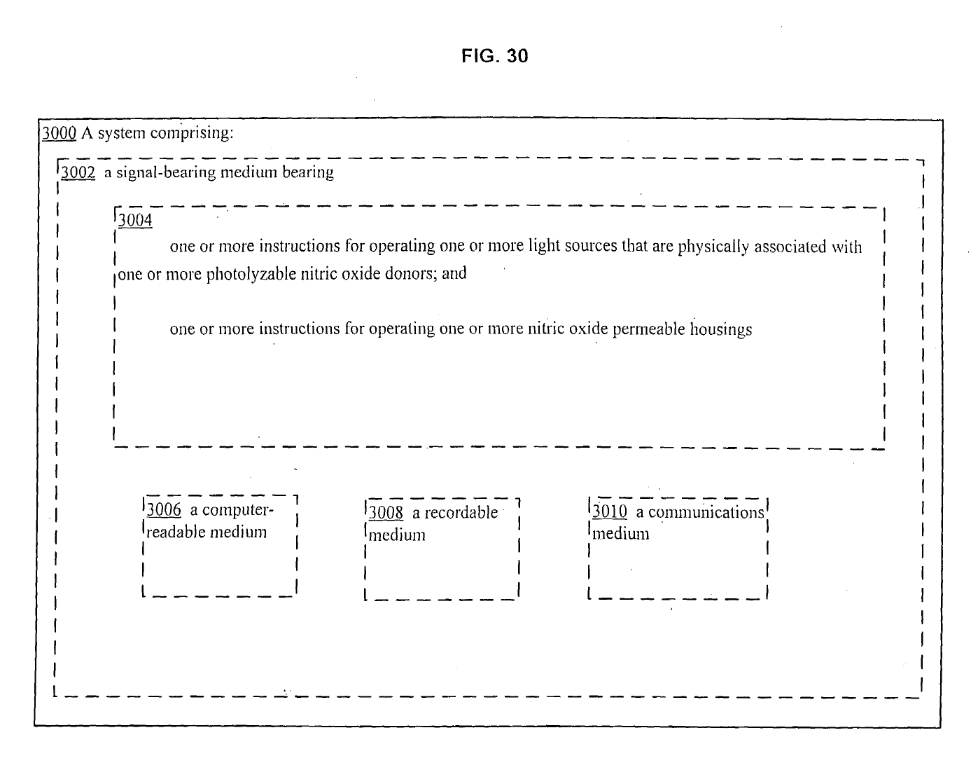

FIG. 30 illustrates a partial view of a system 3000 that includes a computer program 3004 for executing a computer process on a computing device. An embodiment of the system 3000 is provided using a signal-bearing medium 3002 bearing at least one of one or more instructions for operating one or more light sources that are physically associated with one or more photolyzable nitric oxide donors and one or more instructions for operating one or more nitric oxide permeable housings. The one or more instructions may be, for example, computer executable and/or logic-implemented instructions. In some embodiments, the signal-bearing medium 3002 may include a computer-readable medium 3006. In some embodiments, the signal bearing medium 3002 may include a recordable medium 3008. In some embodiments, the signal bearing medium 3002 may include a communications medium 3010.

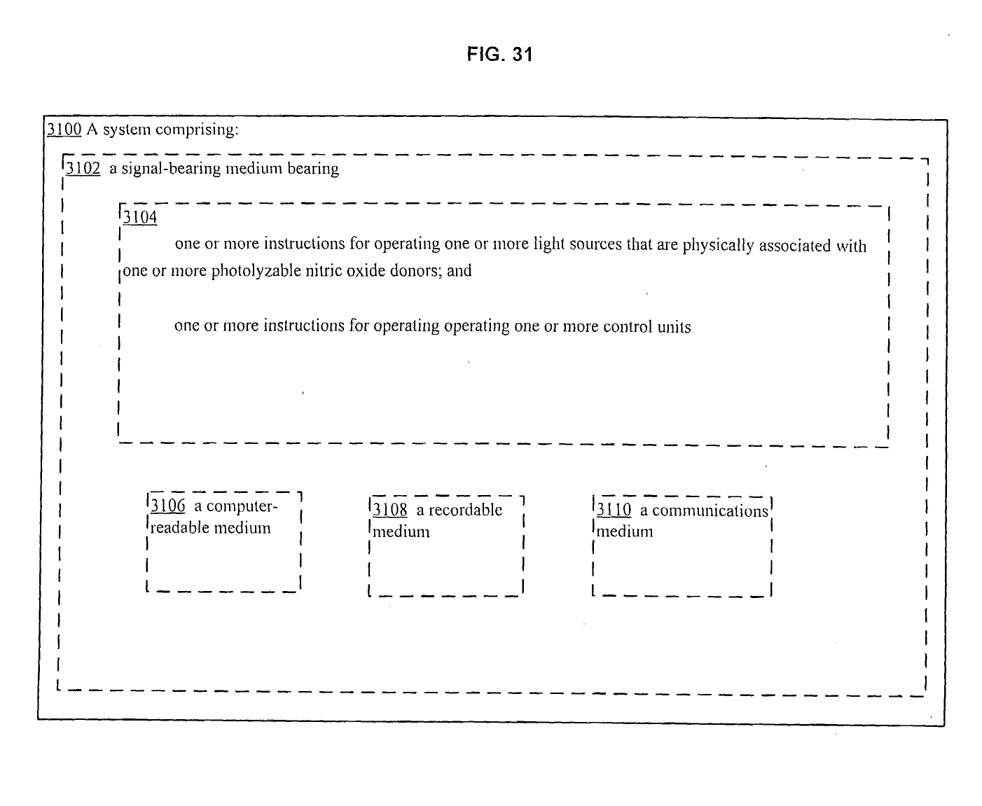

FIG. 31 illustrates a partial view of a system 3100 that includes a computer program 3104 for executing a computer process on a computing device. An embodiment of the system 3100 is provided using a signal-bearing medium 3102 bearing at least one of one or more instructions for operating one or more light sources that are physically associated with one or more photolyzable nitric oxide donors and one or more instructions for operating operating one or more control units. The one or more instructions may be, for example, computer executable and/or logic-implemented instructions. In some embodiments, the signal-bearing medium 3102 may include a computer-readable medium 3106. In some embodiments, the signal bearing medium 3102 may include a recordable medium 3108. In some embodiments, the signal bearing medium 3102 may include a communications medium 3110.

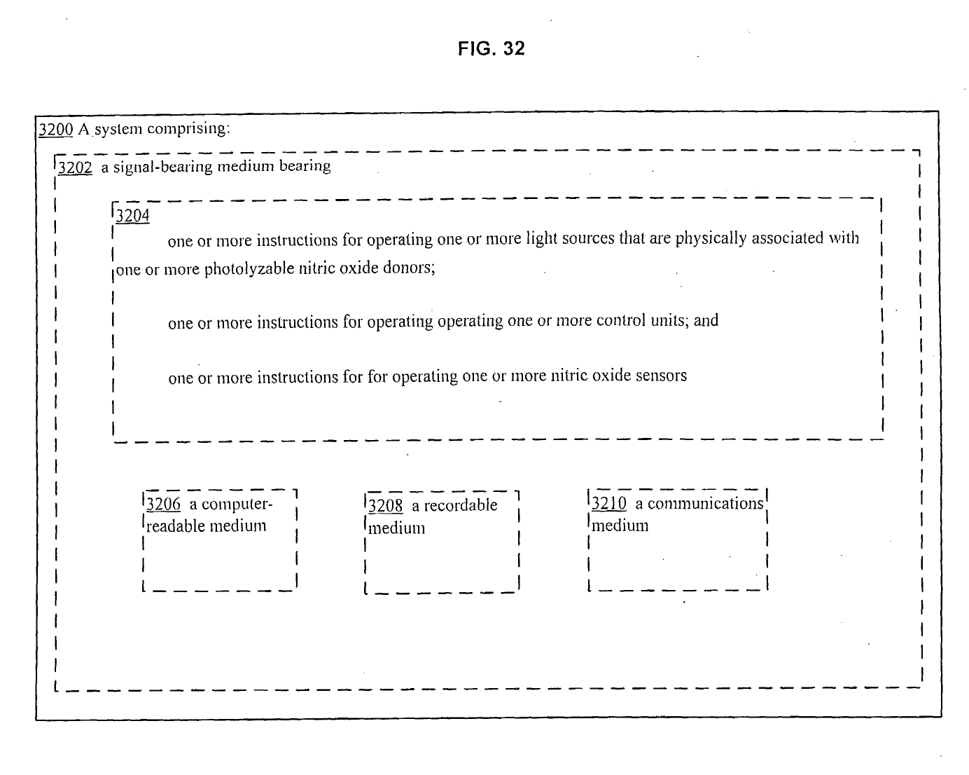

FIG. 32 illustrates a partial view of a system 3200 that includes a computer program 3204 for executing a computer process on a computing device. An embodiment of the system 3200 is provided using a signal-bearing medium 3202 bearing at least one of one or more instructions for operating one or more light sources that are physically associated with one or more photolyzable nitric oxide donors, one or more instructions for operating operating one or more control units, and one or more instructions for operating one or more nitric oxide sensors. The one or more instructions may be, for example, computer executable and/or logic-implemented instructions. In some embodiments, the signal-bearing medium 3202 may include a computer-readable medium 3206. In some embodiments, the signal bearing medium 3202 may include a recordable medium 3208. In some embodiments, the signal bearing medium 3202 may include a communications medium 3210.

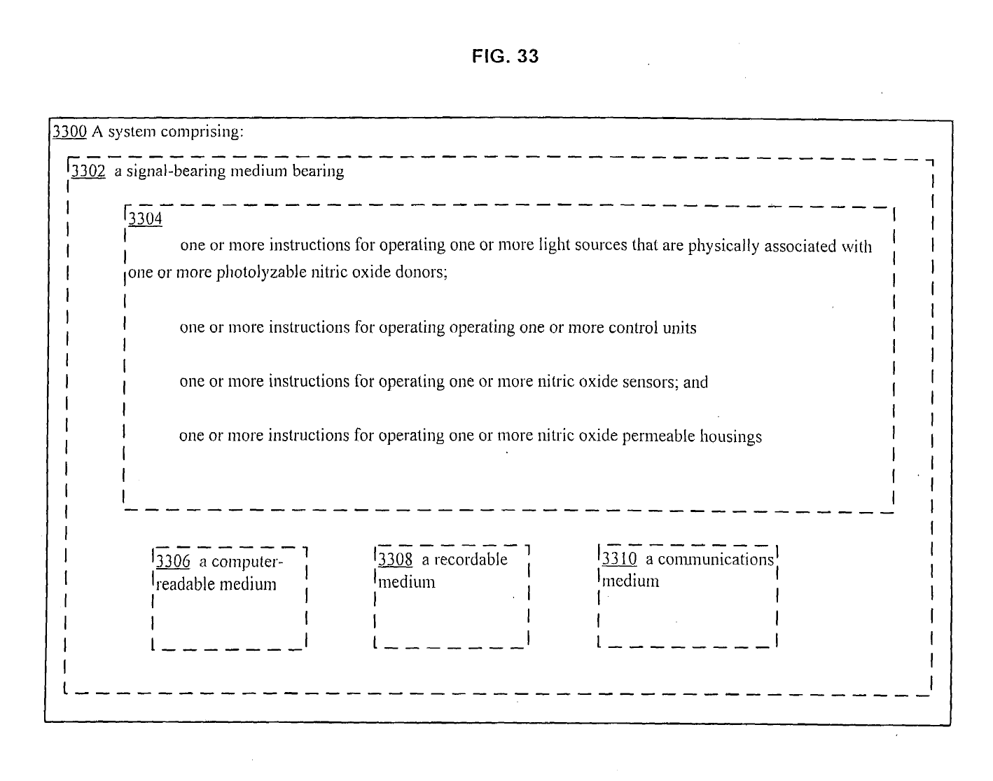

FIG. 33 illustrates a partial view of a system 3300 that includes a computer program 3304 for executing a computer process on a computing device. An embodiment of the system 3300 is provided using a signal-bearing medium 3302 bearing at least one of one or more instructions for operating one or more light sources that are physically associated with one or more photolyzable nitric oxide donors, one or more instructions for operating operating one or more control units, and one or more instructions for operating one or more nitric oxide sensors. The one or more instructions may be, for example, computer executable and/or logic-implemented instructions. In some embodiments, the signal-bearing medium 3302 may include a computer-readable medium 3306. In some embodiments, the signal bearing medium 3302 may include a recordable medium 3308. In some embodiments, the signal bearing medium 3302 may include a communications medium 3310.

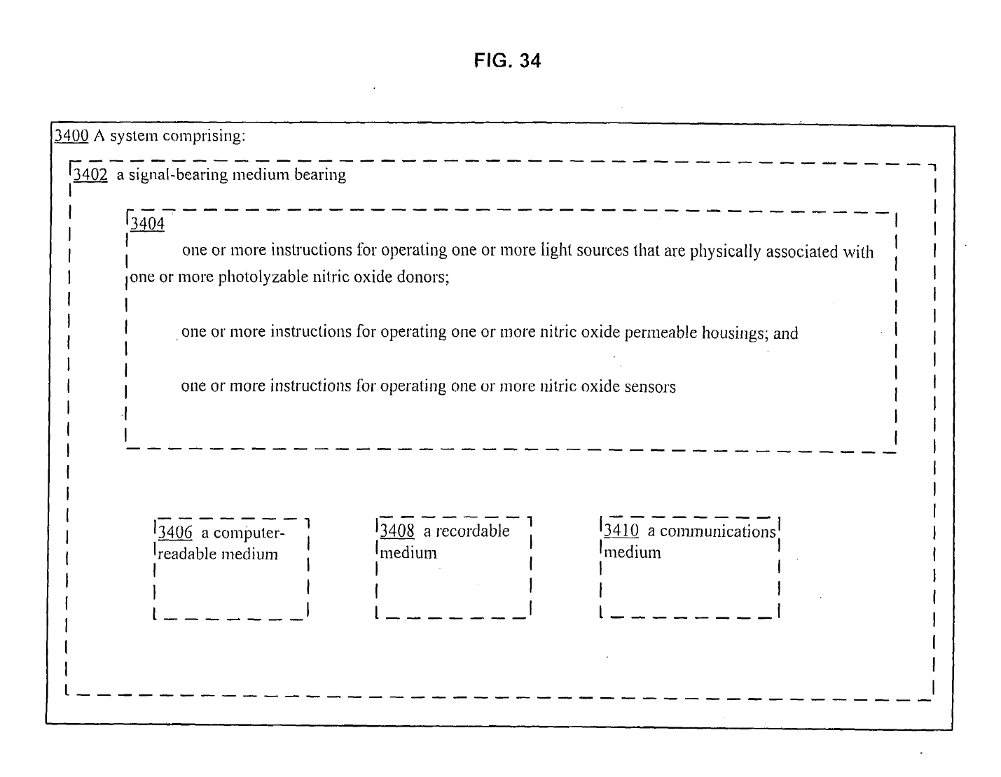

FIG. 34 illustrates a partial view, of a system 3400 that includes a computer program 3404 for executing a computer process on a computing device. An embodiment of the system 3400 is provided using a signal-bearing medium 3402 bearing at least one of one or more instructions for operating one or more light sources that are physically associated with one or more photolyzable nitric oxide donors, one or more instructions for operating one or more nitric oxide permeable housings, and one or more instructions for operating one or more nitric oxide sensors. The one or more instructions may be, for example, computer executable and/or logic-implemented instructions. In some embodiments, the signal-bearing medium 3402 may include a computer-readable medium 3406. In some embodiments, the signal bearing medium 3402 may include a recordable medium 3408. In some embodiments, the signal bearing medium 3402 may include a communications medium 3410.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal-bearing medium used to actually carry out the distribution. Examples of a signal-bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

In a general sense, those skilled in the art will recognize that the various embodiments described herein can be implemented, individually and/or collectively, by various types of electromechanical systems having a wide range of electrical components such as hardware, software, firmware, or virtually any combination thereof; and a wide range of components that may impart mechanical force or motion such as rigid bodies, spring or torsional bodies, hydraulics, and electro-magnetically actuated devices, or virtually any combination thereof. Consequently, as used herein “electro-mechanical system” includes, but is not limited to, electrical circuitry operably coupled with a transducer (e.g., an actuator, a motor, a piezoelectric crystal, etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), electrical circuitry forming a communications device (e.g., a modem, communications switch, or optical-electrical equipment), and any non-electrical analog thereto, such as optical or other analogs. Those skilled in the art will also appreciate that examples of electro-mechanical systems include but are not limited to a variety of consumer electronics systems, as well as other systems such as motorized transport systems, factory automation systems, security systems, and communication/computing systems. Those skilled in the art will recognize that electro-mechanical as used herein is not necessarily limited to a system that has both electrical and mechanical actuation except as context may dictate otherwise.

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

Those skilled in the art will recognize that it is common within the art to implement devices and/or processes and/or systems in the fashion(s) set forth herein, and thereafter use engineering and/or business practices to integrate such implemented devices and/or processes and/or systems into more comprehensive devices and/or processes and/or systems. That is, at least a portion of the devices and/or processes and/or systems described herein can be integrated into other devices and/or processes and/or systems via a reasonable amount of experimentation. Those having skill in the art will recognize that examples of such other devices and/or processes and/or systems might include—as appropriate to context and application—all or part of devices and/or processes and/or systems of (a) an air conveyance (e.g., an airplane, rocket, hovercraft, helicopter, etc.), (b) a ground conveyance (e.g., a car, truck, locomotive, tank, armored personnel carrier, etc.), (c) a building (e.g., a home, warehouse, office, etc.), (d) an appliance (e.g., a refrigerator, a washing machine, a dryer, etc.), (e) a communications system (e.g., a networked system, a telephone system, a voice-over IP system, etc.), (f) a business entity (e.g., an Internet Service Provider (ISP) entity such as Comcast Cable, Quest, Southwestern Bell, etc), or (g) a wired/wireless services entity (e.g., such as Sprint, Cingular, Nextel, etc.), etc.

Although the user interface 124 is shown/described herein as a single illustrated figure that is associated with an individual 126, those skilled in the art will appreciate that a user interface 124 may be utilized by a user that is a representative of a human user, a robotic user (e.g., computational entity), and/or substantially any combination thereof (e.g., a user may be assisted by one or more robotic based systems). In addition, a user as set forth herein, although shown as a single entity may in fact be composed of two or more entities. Those skilled in the art will appreciate that, in general, the same may be said of “sender” and/or other entity-oriented terms as such terms are used herein.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

All publications, patents and patent applications cited herein are incorporated herein by reference. The foregoing specification has been described in relation to certain embodiments thereof, and many details have been set forth for purposes of illustration, however, it will be apparent to those skilled in the art that the invention is susceptible to additional embodiments and that certain of the details described herein may be varied considerably without departing from the basic principles of the invention.

1.-74. (canceled)

75. A system comprising:
circuitry for operating one or more light sources that are physically associated with one or more photolyzable nitric oxide donors.

76. The system of claim 75, wherein the circuitry for operating one or more light sources that are physically associated with one or more photolyzable nitric oxide donors comprises:
circuitry for operating one or more light emitters.

77. The system of claim 75, wherein the circuitry for operating one or more light sources that are physically associated with one or more photolyzable nitric oxide donors comprises:
circuitry for operating one or more power supplies.

78. The system of claim 75, wherein the circuitry for operating one or more light sources that are physically associated with one or more photolyzable nitric oxide donors comprises:
circuitry for operating one or more electromagnetic receivers.

79. The system of claim 75, wherein the circuitry for operating one or more light sources that are physically associated with one or more photolyzable nitric oxide donors comprises:
circuitry for operating one or more light emitting diodes.

80.-81. (canceled)

82. The system of claim 75, wherein the circuitry for operating one or more light sources that are physically associated with one or more photolyzable nitric oxide donors comprises:
circuitry for operating the one or more light sources that are associated with one or more optical waveguides.

83. The system of claim 75, wherein the circuitry for operating one or more light sources that are physically associated with one or more photolyzable nitric oxide donors comprises:
circuitry for operating the one or more light sources that are associated with one or more quantum dots.

84. (canceled)

85. The system of claim 75, wherein the circuitry for operating one or more light sources that are physically associated with one or more photolyzable nitric oxide donors comprises:
circuitry for operating the one or more light sources that are associated with one or more rare-earth materials.

86.-87. (canceled)

88. The system of claim 75, wherein the circuitry for operating one or more light sources that are physically associated with one or more photolyzable nitric oxide donors comprises:
circuitry for operating the one or more light sources that emit visible light.

89. The system of claim 75, wherein the circuitry for operating one or more light sources that are physically associated with one or more photolyzable nitric oxide donors comprises:
circuitry for operating the one or more light sources that emit infrared light.

90. The system of claim 75, wherein the circuitry for operating one or more light sources that are physically associated with one or more photolyzable nitric oxide donors comprises:
circuitry for operating the one or more light sources that are configured to emit light that specifically facilitates release of nitric oxide from the one or more nitric oxide donors.

91. The system of claim 75, wherein the circuitry for operating one or more light sources that are physically associated with one or more photolyzable nitric oxide donors comprises:
circuitry for operating the one or more light sources that are configured to emit light that is selected to avoid damaging one or more tissues.

92. The system of claim 75, further comprising:
circuitry for operating one or more nitric oxide permeable housings.

93. The system of claim 92, wherein the circuitry for operating one or more nitric oxide permeable housings comprises:
circuitry for operating the one or more nitric oxide permeable housings that include one or more controllable valves.

94. (canceled)

95. The system of claim 75, further comprising:
circuitry for operating one or more control units.

96. The system of claim 95, wherein the circuitry for operating one or more control units comprises:
circuitry for operating the one or more control units that are operably associated with the one or more light sources.

97.-98. (canceled)

99. The system of claim 95, wherein the circuitry for operating one or more control units comprises:
circuitry for operating one or more receivers that are configured to receive one or more signals from one or more sensors.

100. The system of claim 95, wherein the circuitry for operating one or more control units comprises:
circuitry for operating the one or more control units that regulate the one or more light sources.

101.-107. (canceled)

108. The system of claim 95, wherein the circuitry for operating one or more control units comprises:
circuitry for operating the one or more control units that are responsive to one or more commands.

109. (canceled)

110. The system of claim 95, wherein the circuitry for operating one or more control units comprises:
circuitry for operating the one or more control units that are associated with one or more transmitters.

111. The system of claim 95, wherein the circuitry for operating one or more control units comprises:
circuitry for operating the one or more control units that include memory.

112. The system of claim 95, wherein the circuitry for operating one or more control units comprises:
circuitry for operating the one or more control units that include memory having one or more associated programs.

113. The system of claim 95, wherein the circuitry for operating one or more control units comprises:
circuitry for operating the one or more control units that regulate one or more associations of one or more light sources with one or more photolyzable nitric oxide donors.

114. The system of claim 95, further comprising:
circuitry for operating one or more nitric oxide sensors.

115. The system of claim 114, wherein the circuitry for operating one or more nitric oxide sensors comprises:
circuitry for operating the one or more nitric oxide sensors that are configured to detect nitric oxide.

116. (canceled)

117. The system of claim 114, wherein the circuitry for operating one or more nitric oxide sensors comprises:
circuitry for operating the one or more nitric oxide sensors that are configured to detect one or more nitric oxide donors.

118. The system of claim 114, wherein the circuitry for operating one or more nitric oxide sensors comprises:
circuitry for operating the one or more nitric oxide sensors that are operably associated with the one or more control units.

119. (canceled)

120. The system of claim 114, wherein the circuitry for operating one or more nitric oxide sensors comprises:
circuitry for operating the one or more nitric oxide sensors that are configured to transmit one or more signals.

121.-125. (canceled)

126. The system of claim 114, further comprising:
circuitry for operating one or more nitric oxide permeable housings.

127. The system of claim 126, wherein the circuitry for operating one or more nitric oxide permeable housings comprises:
circuitry for operating the one or more nitric oxide permeable housings that include one or more controllable valves.

128. (canceled)

129. The system of claim 126, wherein the circuitry for operating one or more nitric oxide permeable housings comprises:
circuitry for operating the one or more nitric oxide permeable housings that are regulated by the one or more control units.

130. The system of claim 92, further comprising:
circuitry for operating one or more nitric oxide sensors.

131. The system of claim 130, wherein the circuitry for operating one or more nitric oxide sensors comprises:
circuitry for operating the one or more nitric oxide sensors that are configured to detect nitric oxide.

132. (canceled)

133. The system of claim 130, wherein the circuitry for operating one or more nitric oxide sensors comprises:
circuitry for operating the one or more nitric oxide sensors that are configured to detect one or more nitric oxide donors.

134. The system of claim 130, wherein the circuitry for operating one or more nitric oxide sensors comprises:
circuitry for operating the one or more nitric oxide sensors that are operably associated with the one or more control units.

135. (canceled)

136. The system of claim 130, wherein the circuitry for operating one or more nitric oxide sensors comprises:
circuitry for operating the one or more nitric oxide sensors that are configured to transmit one or more signals.

137.-141. (canceled)

142. A system comprising:
means for operating one or more light sources that are physically associated with one or more photolyzable nitric oxide donors.

143. The system of claim 142, further comprising:
means for operating one or more nitric oxide permeable housings.

144. The system of claim 142, further comprising:
means for operating one or more control units.

145. The system of claim 144, further comprising:
means for operating one or more nitric oxide sensors.

146. The system of claim 145, further comprising:
means for operating one or more nitric oxide permeable housings.

147. The system of claim 143, further comprising:
means for operating one or more nitric oxide sensors.

148. A system comprising:
a signal-bearing medium bearing:
one or more instructions for operating one or more light sources that are physically associated with one or more photolyzable nitric oxide donors.

149.-151. (canceled)

152. The system of claim 148, further comprising:
one or more instructions for operating one or more nitric oxide permeable housings.

153. The system of claim 148, further comprising:
one or more instructions for operating one or more control units.

154. The system of claim 153, further comprising:
one or more instructions for operating one or more nitric oxide sensors.

155. The system of claim 154, further comprising:
one or more instructions for operating one or more nitric oxide permeable housings.

156. The system of claim 152, further comprising:
one or more instructions for operating one or more nitric oxide sensors.
